SEQUENCE LISTING (13 sequences)

SEQ ID NO: 1 - DNA, 7237 bp, unidentified
  source 1..7237: mol_type = other DNA
acgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaaagcttctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtacccggatccgggcccttcgaacaaaaactcatctcagaagaggatctggtcgactagcgtacggtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaa

SEQ ID NO: 2 - DNA, 4343 bp, synthetic construct
  source 1..4343: mol_type = other DNA
ctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtacccggatccgggcccttcgaacaaaaactcatctcagaagaggatctggtcgactagcgtacggtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgat

SEQ ID NO: 3 - DNA, 1050 bp, synthetic construct
  source 1..1050: mol_type = other DNA
ctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaag

SEQ ID NO: 4 - DNA, 1836 bp, synthetic construct
  source 1..1836: mol_type = other DNA
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctag

SEQ ID NO: 5 - DNA, 1400 bp, synthetic construct
  source 1..1400: mol_type = other DNA
gtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgat

SEQ ID NO: 6 - protein, 611 aa, Homo sapiens
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVP

SEQ ID NO: 7 - DNA, 4117 bp, synthetic construct
  source 1..4117: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtacccggatccgggcccttcgaacaaaaactcatctcagaagaggatctggtcgactagcgtacgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaactagtagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccagcctgacctagctcctggactgaccactattggagcctctcctactcagactgttactctggtgacacaacctgtggttactaaggaaactgccatctccaaactagaaatgccatcttccttgatgttggaggtacctactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaa

SEQ ID NO: 8 - DNA, 8922 bp, synthetic construct
  source 1..8922: mol_type = other DNA
tgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacattattgaagcatttatcagggttattgtctcagacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagtctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctgaggatcccccagtacatgaccttatggcctagggtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgc

SEQ ID NO: 9 - DNA, 4318 bp, synthetic construct
  source 1..4318: mol_type = other DNA
ctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctgaggatcccccagtacatgaccttatggcctagggtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgat

SEQ ID NO: 10 - DNA, 8821 bp, synthetic construct
  source 1..8821: mol_type = other DNA
tgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacattattgaagcatttatcagggttattgtctcagacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagtttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctgaggatcccccagtacatgaccttatggcctagggtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgc

SEQ ID NO: 11 - DNA, 4217 bp, synthetic construct
  source 1..4217: mol_type = other DNA
ttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctgaggatcccccagtacatgaccttatggcctagggtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgt

SEQ ID NO: 12 - DNA, 734 bp, synthetic construct
  source 1..734: mol_type = other DNA
ttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaag

SEQ ID NO: 13 - DNA, 1615 bp, synthetic construct
  source 1..1615: mol_type = other DNA
gtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaaccttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgt

(19) United States
(12) Patent Application Publication
(10) Pub. No.: US 2025/0242053 A1
(43) Pub. Date: Jul. 31, 2025

(54) RECOMBINANT THERAPEUTIC FMR1 CONSTRUCTS AND METHODS OF TREATING FRAGILE X SYNDROME AND RELATED DISORDERS

(71) Applicant: THE UNIVERSITY COURT OF THE UNIVERSITY OF EDINBURGH, Edinburgh, Lothian (GB)

(72) Inventors: Stuart Robert Cobb, Edinburgh, Lothian (GB); Ralph David Hector, Edinburgh, Lothian (GB); Paul Ross, Edinburgh, Lothian (GB)

(21) Appl. No.: 18/853,310

(22) PCT Filed: Apr. 26, 2023
(86) PCT No.: PCT/GB2023/051099

§ 371 Date: Oct. 1, 2024

(30) Foreign Application Priority Data
Apr. 29, 2022 (GB) 2206336.6

(51) Int. Cl. A61K 48/00; A61K 9/00; A61K 38/17; A61P 25/28; C12N 15/86

(52) U.S. Cl. CPC A61K 48/005; A61K 9/0085; A61K 38/1709; A61K 48/0075; A61P 25/28; C12N 15/86; C12N 2750/14143; C12N 2830/50

(57) ABSTRACT

Recombinant human FMR1 constructs and related methods for treating Fragile X syndrome and related disorders in a subject are provided.

### CROSS-REFERENCE TO RELATED APPLICATIONS

This application claims the benefit of and priority to United Kingdom Patent Application No. GB 2206336.6, filed on Apr. 29, 2022, which is hereby incorporated by reference in its entirety.

### SEQUENCE LISTING

The instant application contains a Sequence Listing with 13 sequences, which has been submitted electronically in XML format and is hereby incorporated herein by reference in its entirety. Said XML copy, created on Apr. 19, 2023, is named 55225WO_CRF_sequencelisting.xml, and is 61,563 bytes.

### BACKGROUND

Fragile X Syndrome (FXS) is an X-linked disorder affecting approximately 1 in 4,000 males and 1 in 8,000 females worldwide. It is the leading inherited cause of intellectual disability and autism. Many FXS patients also experience attention deficit hyperactivity disorder (ADHD), increased seizure susceptibility, anxiety, and language difficulties. FXS results from expansion of a CGG trinucleotide repeat within the 5′ untranslated region (UTR) of the Fragile X gene, FMR1.

The normal length of this locus is between 5 and 44 CGG repeats. However, longer stretches of CGG repeats are prone to instability when inherited from parent to child. A premutation allele (55-200 CGG repeats) is the causative mutation of Fragile X-associated Tremor/Ataxia Syndrome (FXTAS), a late-onset neurodegenerative disorder, and Fragile X-associated Primary Ovarian Insufficiency (FXPOI), a condition that results in fertility issues in females. A full mutation (greater than 200 CGG repeats) is the predominant cause of Fragile X syndrome (FXS). FXS is the most common single gene cause of intellectual disability and autism spectrum disorders. Individuals with a FMR1 premutation are at risk of developing FXTAS or FXPOI (1:130-1:256 females and 1:250-1:810 males) and individuals with a full mutation and are diagnosed with FXS (1:2500 to 1:8000 females; 1:5000 males).

When a premutation allele is present on the FMR1 gene, it is transcribed into the messenger RNA (mRNA). However, it is upstream of the start codon and will not be translated as part of the canonical protein isoforms. While the mechanisms resulting in FXTAS and FXPOI are not fully understood, the longer CGG repeat stretch is thought to alter how the gene is expressed (higher mRNA levels, and alternative translation initiation) and how other proteins interact with the CGG repeat or alternative FMRP isoform. Alternatively, when a full mutation allele is present on the FMR1 gene, it undergoes epigenetic changes including methylation of the cytosine nucleotides within the CGG repeat and along the promoter region, and modification of histones to have a heterochromatin signature. These changes result in FMR1 silencing, such that the gene is neither significantly transcribed into mRNA nor significantly translated into protein. Although the exact mechanism and order of events leading to transcriptional silencing remains incompletely understood, the net result of these epigenetic alterations is the absence of the Synaptic functional regulator FMR1, also known as Fragile X Mental Retardation Protein (FMRP).

There is strong evidence that loss of FMRP causes FXS symptoms, as rare patients with mutations or deletions elsewhere in FMR1 also present with FXS. Moreover, Fmr1 knockout (KO) mouse models recapitulate many key features of the human disease, including learning deficits, abnormal socialization and anxiety behaviors, enhanced seizure susceptibility and dendritic spine morphologic abnormalities. FMRP is an RNA-binding protein that binds ˜4% of brain mRNAs, including an enriched fraction of synaptic transcripts from genes associated with autism.

There currently are no effective treatments for FXS and related disorder and thus, there is a need in the field for an effective therapy for treating these disorders and restoring FMRP production.

### SUMMARY

FXS is considered a good candidate for gene therapy as it is a monogenic disorder with severe manifestations and has no effective treatment. A goal of the present disclosure was to develop effective gene therapy cassettes for treating FXS. In an aspect, the present disclosure provides a therapeutic polynucleotide comprising:
a human endogenous FMR1 promoter fragment; a human FMR1 isoform, suitably human FMR1 isoform 7 sequence, an endogenous human 3′ regulatory element, and a polyadenylation signal.

In embodiments, the human FMR1 coding sequence comprises the nucleotide sequence of SEQ ID NO:4, or a nucleotide sequence having at least 80%, 90%, 95% or 99% identity to SEQ ID NO:4. In embodiments, the human FMR1 coding sequence comprises the nucleotide sequence of SEQ ID NO:4, or a nucleotide sequence lacking exon 12, in particular lacking exon 12 and having at least 80%, at least 90%, at least 95% identity to SEQ ID NO:4. In embodiments, the human FMR1 coding sequence comprises a nucleotide sequence encoding a functionally equivalent nucleotide sequence of SEQ ID NO:4. In some embodiments, the human FMR1 coding sequence encodes polypeptide comprising an amino acid sequence having at least 80%, at least 90%, at least 95% identity to SEQ ID NO:6 or a human FMRP.

In embodiments, the promoter comprises SEQ ID NO:3 or SEQ ID NO:12 or a nucleotide sequence having at least 80%, 90%, 95% or 99% identity to SEQ ID NO:3 or SEQ ID NO: 12. In certain embodiments, the 3′regulatory element comprises SEQ ID NO:5 or SEQ ID NO: 13 or a nucleotide sequence having at least 80%, 90%, 95% or 99% identity to SEQ ID NO: 5 or SEQ ID NO:13. Suitably a promoter and/or the 3′ regulatory element that is functionally equivalent to SEQ ID NO:3, SEQ ID NO: 5, SEQ ID NO: 12, or SEQ ID NO: 13 may be provided.

In embodiments, the therapeutic construct sequence comprises the nucleotide sequence of SEQ ID NO:2, SEQ ID NO:9, or SEQ ID NO:11, or a nucleotide sequence having at least 90%, 95% or 99% identity to SEQ ID NO:2, SEQ ID NO:9, or SEQ ID NO:11.

In embodiments, the polynucleotide further comprising at least one adeno-associated virus (AAV) inverted terminal repeat (ITR).

In embodiments, the polynucleotide comprises two AAV ITRs.

In additional embodiments, the disclosure provides a vector comprising the polynucleotide of any of the embodiments described herein.

In embodiments, the vector is a viral vector.

In embodiments, the vector is an adeno-associated virus (AAV) vector.

In embodiments, the AAV vector is an AAV9 vector.

In another aspect, the present disclosure provides a recombinant adeno-associated virus (rAAV), comprising any of the polynucleotides or vectors described herein. In certain embodiments, the rAAV is AAV9.

In another aspect, the present disclosure provides a virion comprising the rAAV described herein.

In another aspect, the present disclosure provides a transformed cell comprising any of the polynucleotides described herein, the vectors described herein, the rAAVs described herein, or the virions described herein.

In another aspect, the present disclosure provides a pharmaceutical composition comprising any of the polynucleotides described herein, the vectors described herein, the rAAVs described herein, or the virions described herein, and optionally, a pharmaceutically acceptable carrier.

In another aspect, the present disclosure provides a method of treating FXS or a FMR1-associated disorder in a subject, the method comprising administering to the subject an effective amount of any of the polynucleotides described herein, the vectors described herein, the rAAVs described herein, or the virions described herein, or the pharmaceutical compositions described herein. As such, there is provided any of the polynucleotides described herein, the vectors described herein, the rAAVs described herein, the virions described herein, or the pharmaceutical compositions described herein, for use as a medicament. In particular, there is provided any of the polynucleotides described herein, the vectors described herein, the rAAVs described herein, the virions described herein, or the pharmaceutical compositions described herein, for use in a method of treating FXS or a FMR1-associated disorder in a subject. Further provided is use of any of the polynucleotides described herein, the vectors described herein, the rAAVs described herein, the virions described herein, or the pharmaceutical compositions described herein in the preparation of a medication for treating FXS or a FMR1-associated disorder in a subject.

### BRIEF DESCRIPTION OF THE DRAWINGS

FIGS. 1A-B. FIG. 1A illustrates the FMR1 construct driven by expression from the constitutive CBh promoter (SEQ ID NO:7), and (FIG. 1B) the low levels of transgenic FMRP expression observed by immunohistochemistry analysis 70 days after injection into P0-2 wild-type mice by ICV delivery. AAV9-CBh-hFMR1-Myc-bGHpA was delivered at two different doses, and levels of FMRP compared to vehicle-treated wild-type mice.

FIG. 2 illustrates the FMR1 construct design of SEQ ID NO:2 incorporating a 1050 bp fragment of the endogenous FMR1 promoter (hFMP1050), a human wild-type FMR1 sequence coding for isoform 7 (hFMR1), a Myc tag, and a fragment of the endogenous 3′UTR of human FMR1 (FMpA1).

FIG. 3 illustrates a plasmid FMR1 construct containing the hMP1050-FMR1-Myc-FMpA1 construct flanked by wild-type AAV2 inverted terminal repeats (ITRs). The plasmid has a sequence of SEQ ID NO: 1.

FIG. 4 shows the experimental design and a schematic of the various phenotyping analysis utilized to test Fmr1^{-/y} mice following dosing with AAV9-hFMP1050-Myc-hFMR1-FMpA1.

FIGS. 5A-B shows marble burying data for WT vs Fmr1^{-/y} mice (FIG. 5A) and an observable therapeutic treatment effect on marble burying in Fmr1^{-/y} mice receiving different doses of AAV9-hFMP1050-Myc-hFMR1-FMpA1 (FIG. 5B).

FIGS. 6A-C illustrates (FIG. 6A) the strong genotype effect observed in the Fmr1^{-/y} mouse model for audiogenic seizures; (FIGS. 6B-C) the therapeutic effect of treatment with AAV9-hFMP1050-Myc-hFMR1-FMpA1 (Virus). Increasing doses of AAV9-hFMP1050-hFMR1-Myc-FMpA1 reduces the seizure incidence in treated Fmr1^{-/y} mice.

FIG. 7 shows immunoblot data from cortex, hippocampus and thalamus of wild-type (WT) and Fmr1^{-/y} (KO) mice dosed with vehicle or 5E10 vg/mouse of AAV9-hFMP1050-hFMR1-Myc-FMpA1.

FIGS. 8A-B show quantification of FMRP expression levels in the cortex of wild-type (FIG. 8A) and Fmr1^{-/y} mice (FIG. 8B) treated with increasing doses of AAV9-hFMP1050-hFMR1-Myc-FMpA1.

FIGS. 9A-H show immunohistochemistry analysis of transgenic FMRP expression in different brain areas in mice 70 days after injection with AAV9-hFMP1050-hFMR1-Myc-FMpA1. Wild-type (WT) and Fmr1^{-/y} mice were treated at P0-2 by ICV delivery of 1E11 vg/mouse.

FIGS. 10A-D shows increased FMRP expression after delivery of AAV9-hFMP1050-hFMR1-Myc-FMpA1 to Fmr1^{-/y} mice. Representative tiled sagittal whole brain micrographs, immunolabelled against FMRP (green fluorescent), from (FIG. 10A) a vehicle-treated WT mouse, (FIG. 10B) a vehicle-treated Fmr1dy mouse, (FIGS. 10 C-D) an Fmr1^{-/y} mouse treated with AAV9-hFMP1050-hFMR1-Myc-FMpA1 at a dose of (FIG. 10 C) 1E11 vg/mouse or (FIG. 10D) 3E11 vg/mouse. Scale bars represent 1 mm.

FIG. 11 illustrates the FMR1 construct design (SEQ ID NO:9) incorporating a 1050 bp fragment of the endogenous FMR1 promoter (hFMP1050), a human wild-type FMR1 sequence coding for isoform 7 (hFMR1), and a fragment of the endogenous 3′UTR of human FMR1 (FMpA1).

FIG. 12 illustrates a plasmid FMR1 construct (SEQ ID NO:8) containing the hMP1050-FMR1-FMpA1 construct flanked by wild-type AAV2 inverted terminal repeats (ITRs).

FIG. 13 shows the FMR1 promoter region in the UCSC Genome Browser. The 1050 bp fragment contains most of the key genetic regulatory elements in this genomic region needed for endogenous expression and regulation of FMR1.

FIG. 14 shows a dotplot, generated by EMBOSS Dotmatcher, that compares the sequences of human (hum) and mouse (mus) in the genomic region upstream of the FMR1 coding region, incorporating the 1050 bp fragment of the promoter.

FIG. 15 illustrates the FMR1 construct design (the hFMP734-hFMR1-FMpA2 construct, SEQ ID NO:11) incorporating a shorter 734 bp fragment of the endogenous FMR1 promoter (hFMP734), a human wild-type FMR1 sequence coding for isoform 7 (hFMR1), and a fragment of the endogenous 3′UTR of human FMR1 (FMpA2).

FIG. 16 illustrates a plasmid FMR1 construct (SEQ ID NO:10) containing the hMP734-FMR1-FMpA2 construct flanked by wild-type AAV2 inverted terminal repeats (ITRs).

FIG. 17 shows immunoblot data of FMRP expression from plasmids containing hFMP1050-hFMR1-FMpA1 or hFMP734-hFMR1-FMpA2. HEK293A cells were transfected with different amounts of plasmid DNA and levels of FMRP were detected using an anti-FMRP antibody. Levels of FMRP were calculated relative to levels of FMRP in untransfected HEK293A cells, and plotted against copies per cell, calculated from levels of input DNA and numbers of cells per well.

### DEFINITIONS

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. The following terms have the meanings given:

AAV “rep” and “cap” genes refer to polynucleotide sequences encoding replication and encapsidation proteins of adeno-associated virus. AAV rep and cap are referred to herein as AAV “packaging genes.”

“AAV” is an abbreviation for adeno-associated virus and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV 8), AAV type 9 (AAV9), avian AAV, bovine AAV, canine AAV, equine AAV, primate AAV, non-primate AAV, and ovine AAV, and modifications, derivatives, or pseudotypes thereof. “Primate AAV” refers to AAV that infect primates, “non-primate AAV” refers to AAV that infect non-primate mammals, “bovine AAV” refers to AAV that infect bovine mammals, etc. In some embodiments, the AAV particle is AAV1, AAV2, rAAV3, AAV4, AAV5, AAV6, AAV7, AAV8, AAV9, AAV 10, AAV11, AAV12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16. In some embodiments, the rAAV particle is a derivative, modification, or pseudotype of AAV1, AAV2, rAAV3, AAV4, AAV5, AAV6, AAV7, AAV8, AAV9, AAV 10, AAV11, AAV 12, AAV 13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

The various serotypes of AAV are attractive for several reasons, most prominently that AAV is believed to be non-pathogenic and that the wildtype virus can integrate its genome site-specifically into human chromosome 19 (Linden et al., 1996, Proc Natl Acad Sci USA 93:11288-11294). The insertion site of AAV into the human genome is called AAVS1. Site-specific integration, as opposed to random integration, is believed to likely result in a predictable long-term expression profile.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC-001829 (AAV-4), U89790 (AAV-4), NC-006152 (AAV-5), AF513851 (AAV-7), AF513852 (AAV-8), and NC-006261 (AAV-8); the disclosures of which are incorporated by reference herein. See also, e.g., Srivistava et al., 1983, J. Virology 45:555; Chiorini et al., 1998, J. Virology 71:6823; Chiorini et al., 1999, J. Virology 73: 1309; Bantel-Schaal et al., 1999, J. Virology 73:939; Xiao et al., 1999, J. Virology 73:3994; Muramatsu et al., 1996, Virology 221:208; Shade et al., 1986, J. Virol. 58:921; Gao et al., 2002, Proc. Nat. Acad. Sci. USA 99: 11854; Moris et al., 2004, Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111.

An “rAAV vector” as used herein refers to an AAV vector comprising a polynucleotide sequence not of AAV origin (i.e., a polynucleotide heterologous to AAV), typically a sequence of interest for the genetic transformation of a cell. In some embodiments, the heterologous polynucleotide may be flanked by at least one, and sometimes by two, AAV inverted terminal repeat sequences (ITRs). The term rAAV vector encompasses both rAAV vector particles and rAAV vector plasmids. A rAAV vector may either be single-stranded (ssAAV) or self-complementary (scAAV). An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as a “rAAV vector particle” or simply an “rAAV vector”. Thus, production of rAAV particle necessarily includes production of rAAV vector, as such a vector is contained within an rAAV particle.

“Vector,” means a recombinant plasmid or virus that comprises a polynucleotide to be delivered into a host cell, either in vitro or in vivo.

“Recombinant,” as used herein means that the vector, polynucleotide, polypeptide or cell is the product of various combinations of cloning, restriction or ligation steps (e.g. relating to a polynucleotide or polypeptide comprised therein), and/or other procedures that result in a construct that is distinct from a product found in nature. A recombinant virus or vector is a viral particle comprising a recombinant polynucleotide. The terms respectively include replicates of the original polynucleotide construct and progeny of the original virus construct.

“Recombinant viral vector” means a recombinant polynucleotide vector comprising one or more heterologous sequences (i.e., polynucleotide sequence not of viral origin).

“Recombinant”, as applied to an AAV particle means that the AAV particle is the product of one or more procedures that result in an AAV particle construct that is distinct from an AAV particle in nature.

“AAV Rep” means AAV replication proteins and analogs thereof.

“AAV Cap” means AAV capsid proteins, VP1, VP2 and VP3 and analogs thereof. In wild type AAV virus, three capsid genes vp1, vp2 and vp3 overlap each other. See, Grieger and Samulski, 2005, J. Virol. 79(15):9933-9944. A single P40 promoter allows all three capsid proteins to be expressed at a ratio of about 1:1:10, vp1, vp2, vp3, respectively, which complement with rAAV production. For the production of recombinant AAV vectors, desired ratio of VP1:VP2:VP3 is in the range of about 1:1:1 to about 1:1:100, preferably in the range of about 1:1:2 to about 1:1:50, more preferably in the range of about 1:1:5 to about 1:1:20. Although the desired ratio of VP1:VP2 is 1:1, the ratio range of VP1:VP2 could vary from 1:50 to 50:1.

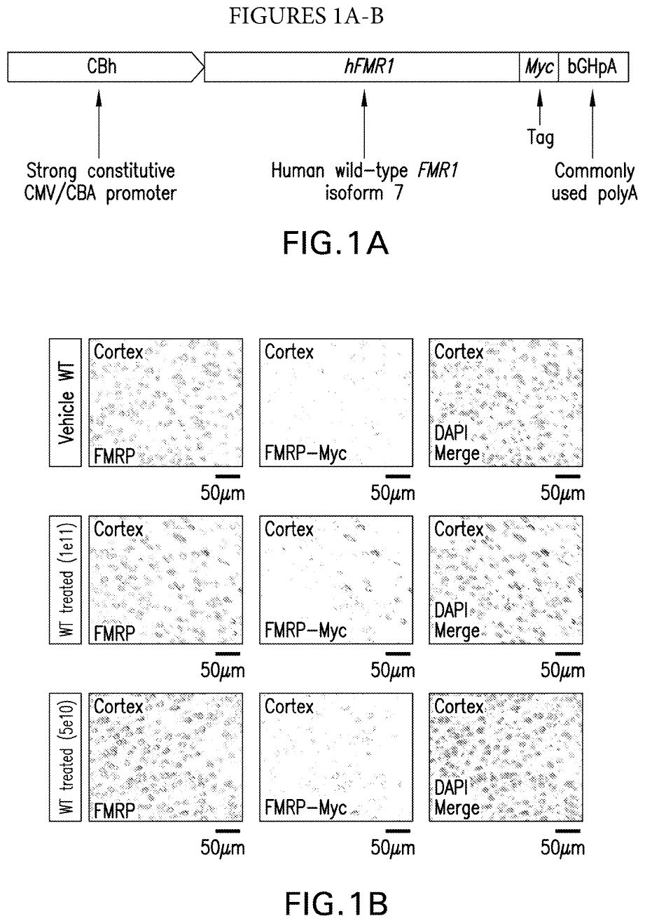

A comprehensive list and alignment of amino acid sequences of capsids of known AAV serotypes is provided by Marsic et al., 2014, Molecular Therapy 22(11):1900-1909, especially at supplementary FIG. 1.

For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in a 1:1:10 ratio in the capsid. That is, for AAVs, VP1 is the full-length protein and VP2 and VP3 are progressively shorter versions of VP1, with increasing truncation of the N-terminus relative to VP1.

“AAV TR” means a palindromic terminal repeat sequence at or near the ends of the AAV genome, comprising mostly complementary, symmetrically arranged sequences, and includes analogs of native AAV TRs and analogs thereof. In the case of recombinant parvovirus vectors, the recombinant polynucleotide is flanked by at least one, preferably two, inverted terminal repeat sequences (ITRs).

“Cis-motifs” includes conserved sequences such as found at or close to the termini of the genomic sequence and recognized for initiation of replication; cryptic promoters or sequences at internal positions likely used for transcription initiation, splicing or termination.

“Therapeutically effective amount” means a minimal amount of active agent which is necessary to impart therapeutic benefit to a subject. For example, a “therapeutically effective amount” to a patient is such an amount which induces, ameliorates, stabilizes, slows down the progression or otherwise causes an improvement in the pathological symptoms, disease progression or physiological conditions associated with or resistance to succumbing to a disorder.

“Gene” means a polynucleotide containing at least one open reading frame that is capable of encoding a particular polypeptide or protein after being transcribed and translated.

“Coding sequence” means a sequence which encodes a particular protein” or “encoding nucleic acid”, denotes a nucleic acid sequence which is transcribed (in the case of DNA) and translated (in the case of mRNA) into a polypeptide in vitro or in vivo when placed under the control of (operably linked to) appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

“Chimeric” means, with respect to a viral capsid or particle, that the capsid or particle includes sequences from different parvoviruses, preferably different AAV serotypes, as described in Rabinowitz et al., U.S. Pat. No. 6,491,907, the disclosure of which is incorporated in its entirety herein by reference. See also Rabinowitz et al., 2004, J. Virol. 78(9):4421-4432. A particularly preferred chimeric viral capsid is the AAV2.5 capsid, which has the sequence of the AAV2 capsid with the following mutations: 263 Q to A; 265 insertion T; 705 N to A; 708 V to A; and 716 T to N. wherein the nucleotide sequence encoding such capsid is defined as SEQ ID NO: 15 as described in WO 2006/066066. Other preferred chimeric AAVs include, but are not limited to, AAV218 described in WO 2010/093784, AAV2G9 and AAV8G9 described in WO 2014/144229, and AAV9.45 (Pulicherla et al., 2011, Molecular Therapy 19(6):1070-1078).

“Flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., TRs), indicates that one element is located 5′ to the sequence and the other is located 3′ to the sequence; however, there may be intervening sequences there between.

“Polynucleotide” means a sequence of nucleotides connected by phosphodiester linkages. Polynucleotides are presented herein in the direction from the 5′ to the 3′ direction. A polynucleotide of the present invention can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U). A polynucleotide of the present invention can be prepared using standard techniques well known to one of skill in the art.

Human FMRP or Synaptic functional regulator FMR1 is described in UniProtKB-Q06787 (FMR1_Human) and is a multifunctional polyribosome-associated RNA-binding protein that plays a central role in neuronal development and synaptic plasticity through the regulation of alternative mRNA splicing, mRNA stability, mRNA dendritic transport and postsynaptic local protein synthesis of a subset of mRNAs.

“Transduction” of a cell by a virus means that there is transfer of a nucleic acid from the virus particle to the cell.

“Transfection” of a cell means that genetic material is introduced into a cell for the purpose of genetically modifying the cell. Transfection can be accomplished by a variety of means known in the art, such as calcium phosphate, polyethyleneimine, electroporation, and the like.

“Polypeptide” encompasses both peptides and proteins, unless indicated otherwise.

“Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

“Transgene” is used to mean any heterologous nucleotide sequence incorporated in a vector, including a viral vector, for delivery to and including expression in a target cell (also referred to herein as a “host cell”), and associated expression control sequences, such as promoters. It is appreciated by those of skill in the art that expression control sequences will be selected based on ability to promote expression of the transgene in the target cell. An example of a transgene is a nucleic acid encoding a therapeutic polypeptide.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

The terms “purifying”, “purification”, “separate”, “separating”, “separation”, “isolate”, “isolating”, or “isolation”, as used herein, refer to increasing the degree of purity of rAAV particles from a sample comprising the target product and one or more impurities. Typically, the degree of purity of the target product is increased by removing (completely or partially) at least one impurity from the sample. In some embodiments, the degree of purity of the rAAV in a sample is increased by removing (completely or partially) one or more impurities from the sample by using a method described herein.

“Homologous” used in reference to peptides, refers to amino acid sequence similarity between two peptides. When an amino acid position in both of the peptides is occupied by identical amino acids, they are homologous at that position. Thus by “substantially homologous” means an amino acid sequence that is largely, but not entirely, homologous, and which retains most or all of the activity as the sequence to which it is homologous.

As used herein, “substantially homologous” as used herein means that a sequence is at least 50% identical, and preferably at least 75% and more preferably 95% homology to the reference peptide. Additional peptide sequence modification are included, such as minor variations, deletions, substitutions or derivatizations of the amino acid sequence of the sequences disclosed herein, so long as the peptide has substantially the same activity or function as the unmodified peptides. Derivatives of an amino acid may include but not limited to trifluoroleucine, hexafluoroleucine, 5,5,5-trifluoroisoleucine, 4,4,4-trifluorovaline, p-fluorophenylaline, o-fluorotyrosine, m-fluorotyrosine, 2,3-difluorotyrosine, 4-fluorohistidine, 2-fluorohistidine, 2,4-difluorohistidine, fluoroproline, difluoroproline, 4-hydroxyproline, selenomethionine, telluromethionine, selenocysteine, selenatryptophans, 4-aminotryptophan, 5-aminotryptophan, 5-hydroxytryptophan, 7-azatryptophan, 4-fluorotryptophan, 5-fluorotryptophan, 6-fluorotryptophan, homoallylglycine, homopropargylglycine, 2-butynylglycine, cis-crotylglycine, allylglycine, dehydroleucine, dehydroproline, 2-amino-3-methyl-4-pentenoic acid, azidohomoalanine, asidoalanine, azidonorleucine, p-ethynylphenylalanine, p-azidophenylalanine, p-bromophenylalanine, p-acetylphenylalanine and benzofuranylalanine. Notably, a modified peptide will retain activity or function associated with the unmodified peptide, the modified peptide will generally have an amino acid sequence “substantially homologous” with the amino acid sequence of the unmodified sequence.

In some embodiments, provided herein are recombinant human FMR1 constructs. Additional embodiments provided herein include nucleic acid constructs, such as vectors, which include as part of their sequence recombinant human FMR1. For example, the invention includes plasmids and/or other vectors that include the recombinant human FMR1 sequence along with other elements, such as regulatory elements. Further, the invention provides packaged gene delivery vehicle, such as a viral capsid, including recombinant human FMR1 sequence. The invention also includes methods of delivery and, preferably, expressing the recombinant human FMR1 gene by delivering the modified sequence into a cell along with elements required to promote expression in the cell. The invention also provides gene therapy methods in which the recombinant human FMR1 gene sequence is administered to a subject, e.g., as a component of a vector and/or packaged as a component of a viral gene delivery vehicle. Particular embodiments include those wherein the recombinant human FMR1 sequence has an identity of 75% to SEQ ID NO: 4 (human FMR1 isoform 7). In certain embodiments the recombinant human FMR1 construct exhibits greater than 75%, 76, 77, 78, 79, 80, 81, 82, 83, 84, 85, 86, 87, 88, 89, 90, 91, 92, 93, 94, 95, 96, 97, 98, or 99% identity with SEQ ID NO: 4 (human FMR1 isoform 7).

Recombinant FMR1 Designs. More than 20 transcript isoforms of human FMR1 have been detected in vivo. Of these, isoform 7 is the most abundant in adult brain tissue. Isoform 7 also lacks exon 12 (due to alternative splicing of human FMR1), and isoforms lacking exon 12 show an increased affinity for kissing complex RNA. Therefore, human FMR1 isoform 7 was selected as the transgene sequence. In the first iteration of a recombinant FMR1 construct design, expression of the transgene was driven by the strong, constitutive promoter CBh (Cytomegalovirus enhancer, Chicken Beta Actin promoter), and the transcript stabilized with the ubiquitous polyadenylation signal bGHpA (bovine growth hormone polyadenylation signal). This sequence is described in SEQ ID NO: 7. Expression of the human FMR1 transgene from this cassette was poor; only just detectable above endogenous levels of FMRP in wild-type treated mice. This led to the design of a new recombinant FMR1 construct design in which expression of the transgene was driven by a fragment of the endogenous human FMR1promoter; a 1050 bp fragment immediately upstream of the Transcription Start Site (TSS) and 5′ UTR of FMR1. This 1050 bp fragment contained high levels of sequence conservation with other species (including mouse and cynomolgus macaque) and incorporated the core promoter elements of the human FMR1 gene, to drive expression of the FMR1 transgene with elements of endogenous regulation (elements absent in a constitutive promoter). The transgene is further regulated by endogenous regulatory elements provided in the 3′UTR. This 1400 bp fragment of the human FMR1 3′UTR contains putative regulatory elements including miRNA recognition sites, and the most used polyadenylation signal by FMR1 transcript isoforms.

Treatment may, for example, be affected to increase levels of FMRP in the subject.

### Modified Nucleic Acid for Expression of FMRP

“Optimized” or “codon-optimized” as referred to interchangeably herein, refer to a coding sequence that has been optimized relative to a wild type coding sequence (e.g., a coding sequence for FMRP) to increase expression of the coding sequence, e.g., by minimizing usage of rare codons, decreasing the number of CpG dinucleotides, removing cryptic splice donor or acceptor sites, removing Kozak sequences, removing ribosomal entry sites, and the like.

“Codon adaptation index” as used herein, refers to the adaption of a codon from a tRNA codon that rarely occurs within the given organism or cell to a tRNA that occurs more often within the given organism or cell. A non-limiting example in which the codon adaptation index is calculated is by using the “GenScript Rare Codon Analysis Tool.” (genscript.com/tools/rare-codon-analysis).

“Percentage identity” as used herein, refers to the numerical score of two given polynucleotides and/or polypeptides that have identical nucleic and/or amino acids within the same position as given by a typical sequence alignment program (i.e. BLAST methods).

### Codon Optimization

There are sixty-four different codons. Sixty-one of them encode the twenty standard amino acids, while another three function as stop codons. The greater number of codons relative to the number of amino acids they code for, means that a single amino acid can be encoded by more than one codon. Indeed, some common amino acids, such as arginine and leucine, are encoded by as many as 6 codons.

Different organisms exhibit bias towards use of certain codons over others for the same amino acid. Some species are known to avoid certain codons almost entirely. Such biases may affect protein expression. Therefore, it is important to consider codon optimization when design gene therapy constructs.

While numerous factors contribute to the success of protein expression, codon optimization plays a critical role, particularly when proteins are expressed in a heterologous system. As an example, if a human gene is to be expressed in E. coli, choosing codons preferentially used by the bacterium can increase the success of protein expression. This is particularly true when rare codons are eliminated.

### Codon Adaptation Index

One option for analyzing codon usage bias is the technique codon adaptation index (CAI). It calculates an index to tell how “foreign” sequence will adapt to the host protein expression machinery. CAI compares the codon usage in the GOI to the most frequent codon usage in a set of highly expressed genes in the model expression organism. The relative adaptiveness of codons ranges from 0 to 1, depending on how the GOI is to the reference set of highly expressed gene sequences, with 1 being the closest.

Examples of reference codon usages are contained in various databases, including the codon usage database kazusa.or.jp/codon/.

### Sequence Modification

Examples of modifications include elimination of one or more cis-acting motifs and introduction of one or more Kozak sequences. In one embodiment, one or more cis-acting motifs are eliminated and one Kozak sequence is introduced.

Examples of cis acting motifs that may be eliminated include internal TATA-boxes; chi-sites; ribosomal entry sites; ARE, INS, and/or CRS sequence elements; repeat sequences and/or RNA secondary structures; (cryptic) splice donor and/or acceptor sites, branch points; and restriction sites, (e.g., Sal1).

Additionally, in certain embodiments, the codon adaptation index of the modified nucleic acid encoding FMRP (i.e., the modified FMR1 gene) is preferably at least 0.74, preferably, at least 0.76, even more preferably, at least 0.77, yet more preferably, at least 0.80, preferably, at least 0.82, more preferably, at least 0.84, yet more preferably, at least 0.85, even more preferably, at least 0.89, yet more preferably, at least 0.90, and most preferably, at least 0.91.

In another embodiment the modified FMR1 sequence has a reduced level of CpG dinucleotides that being a reduction of about 10%, 20%, 30%, 50% or more, compared with the wild-type nucleic acid sequence encoding FMR1 (e.g., SEQ ID NO:4).

It is known that methylation of CpG dinucleotides plays an important role in the regulation of gene expression in eukaryotes. Specifically, methylation of CpG dinucleotides in eukaryotes essentially serves to silence gene expression through interfering with the transcriptional machinery. As such, because of the gene silencing evoked by methylation of CpG motifs, the nucleic acids and vectors of certain embodiments having a reduced number of CpG dinucleotides will provide for high and long-lasting transgene expression level, and potentially less risk of host/subject immunological response and/or toxicity.

In one embodiment, the modified recombinant human FMR1 gene comprises fewer potential CpG dinucleotides than wild type FMR1 gene.

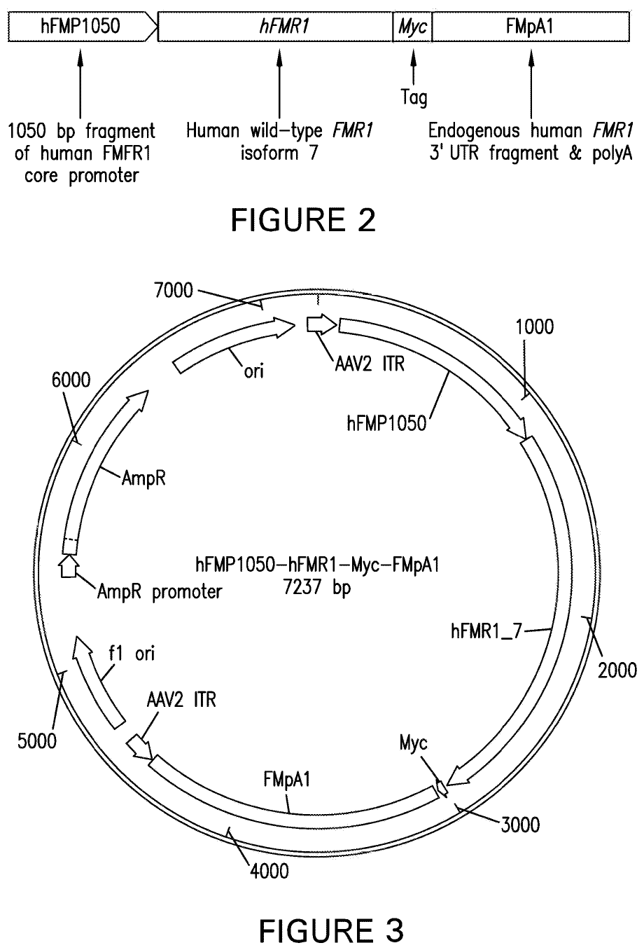

In certain embodiments, the recombinant human FMR1 gene sequence may also include flanking restriction sites to facilitate subcloning into expression vector. Many such restriction sites are well known in the art, and include, but are not limited to, those shown in FIG. 2 (plasmid map of hFMP1050-hFMR1-Myc-FMpA1).

The disclosure includes a nucleic acid vector including the recombinant human FMR1 isoform 7 gene sequence and various regulatory or control elements. The precise nature of regulatory elements useful for gene expression will vary from organism to organism and from cell type to cell type.

In particular embodiments, the promoter comprises a 1050 bp fragment of human core promoter for human FMR1 SEQ ID NO:3) (e.g. a fragment of the endogenous or “native” core promoter for human FMR1). Suitably promoters functionally equivalent to a 1050 bp fragment of human core promoter for human FMR1 may be provided.

In certain embodiments, the promoter comprises a 734 bp fragment of human core promoter for human FMR1 (SEQ ID NO:12) (e.g. a fragment of the endogenous or “native” core promoter for human FMR1). Suitably promoters functionally equivalent to a 734 bp fragment of human core promoter for human FMR1 may be provided, as well as those exhibiting 90, 91, 92, 93, 94, 95, 96, 97, 98, 99% identity thereto.

In general, a promoter which directs the initiation of RNA transcription in the cell of interest is utilized. The promoter may be constitutive or regulated. Constitutive promoters are those which cause an operably linked gene to be expressed essentially at all times. Regulated promoters are those which can be activated or deactivated. Regulated promoters include inducible promoters, which are usually “off” but which may be induced to turn “on,” and “repressible” promoters, which are usually “on” but may be turned “off.” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

Examples of suitable promoters include adenoviral promoters, such as the adenoviral major late promoter; heterologous promoters, such as the cytomegalovirus (CMV) promoter; the respiratory syncytial virus promoter; the Rous Sarcoma Virus (RSV) promoter; the albumin promoter; inducible promoters, such as the Mouse Mammary Tumor Virus (MMTV) promoter; the metallothionein promoter; heat shock promoters; the α-1-antitrypsin promoter; the hepatitis B surface antigen promoter; the transferrin promoter; the apolipoprotein A-1 promoter; chicken beta-actin (CBA) promoter, the CBh promoter, and the CAG promoter (cytomegalovirus early enhancer element and the promoter, the first exon, and the first intron of chicken beta-actin gene and the splice acceptor of the rabbit beta-globin gene) (Alexopoulou et al., 2008, BioMed. Central Cell Biol. 9:2), and human FMR1 promoters. The promoter may be a tissue-specific promoter, such as the mouse albumin promoter, which is active in liver cells as well as the transthyretin promoter (TTR). In certain embodiments, liver detargeted promoters can be used. It will be clear to one skilled in the art how to utilize and adapt any of these features as described herein.

In another aspect, the recombinant human FMR1 construct further comprises an enhancer to increase expression of the protein. Many enhancers are known in the art, including, but not limited to, the cytomegalovirus major immediate-early enhancer. More specifically, the CMV MIE promoter comprises three regions: the modulator, the unique region and the enhancer (Isomura and Stinski, 2003, J. Virol. 77(6):3602-3614). The CMV enhancer region can be combined with other promoters, or a portion thereof, to form hybrid promoters to further increase expression of a nucleic acid operably linked thereto. For example, a chicken beta-actin (CBA) promoter, or a portion thereof, can be combined with the CMV promoter/enhancer, or a portion thereof, and a hybrid intron of chicken beta-actin (CBA) and minute virus of mice (MMV) introns to make a version of CBA termed the “CBh” promoter, which stands for chicken beta-actin hybrid promoter, as described in Gray et al. (2011, Human Gene Therapy 22:1143-1153).

Introns can also be used to increase efficiency in mammalian expression vectors. Examples of introns are murine cytomegalovirus (MCMV) immediate early (IE) promoter, human cytomegalovirus (HCMV) immediate early (IE) promoter, and human elongation factor one alpha (EF-1 alpha) promoter. The intron can be varied depending on the gene of interest.

Further, the control elements can include a collagen stabilization sequence (CSS), a stop codon, a termination sequence, and a poly-adenylation signal sequence, such as, but not limited to a bovine growth hormone poly A signal sequence (bGHpA), to drive efficient addition of a poly-adenosine “tail” at the 3′ end of a eukaryotic mRNA (see, e.g., Goodwin and Rottman, 1992, J. Biol. Chem. 267(23):16330-16334).

In particular embodiments, the recombinant construct comprises an endogenous fragment of the human 3′UTR and polyadenylation sequence (SEQ ID NO: 5, or SEQ ID NO: 13).

The poly-A tail is a long chain of adenine nucleotides that is added to a messenger RNA (mRNA) molecule during RNA processing to increase the stability of the molecule. Similar to what happens in vivo. The poly-A tail makes the RNA molecule more stable and prevents its degradation. Additionally, the poly-A tail allows the mature messenger RNA molecule to be exported from the nucleus and translated into a protein by ribosomes in the cytoplasm.

The woodchuck hepatitis virus post-transcriptional regulatory element (WPRE) increases transgene expression from a variety of viral vectors. WPRE is most effective when placed downstream of the transgene, proximal to the polyadenylation signal. It is possible that WPRE reduces viral mRNA readthrough transcription by improving transcript termination, which in turn would increase viral titers and expression. (Gene Therapy volume 14, pages 1298-1304 (2007)).

### Non-Viral Vectors

In a particular embodiment, the vector used according to the invention is a non-viral vector. Typically, the non-viral vector may be a plasmid which includes nucleic acid sequences containing recombinant human FMR1, or variants thereof.

### Packaged Recombinant FMR1 Sequence

The recombinant human FMR1 gene sequence may also be provided as a component of a packaged viral vector. In general, packaged viral vectors include a viral vector packaged in a capsid. Viral vectors and viral capsids are discussed in the ensuing sections. The nucleic acid packaged in the rAAV vector can be single-stranded (ss), self-complementary (sc), or double-stranded (ds). It is expected that any of the constructs comprising any of SEQ ID NO: 2, SEQ ID NO: 9, or SEQ ID NO:11 is capable of desired packaging and expression.

### Viral Vector

Typically, viral vectors carrying transgenes are assembled from polynucleotides encoding the transgene, suitable regulatory elements and elements necessary for production of viral proteins which mediate cell transduction. Examples of a viral vector include but are not limited to adenoviral, retroviral, lentiviral, herpesvirus and adeno-associated virus (AAV) vectors.

The viral vector component of the packaged viral vectors produced according to the methods of the invention includes at least one transgene, e.g., recombinant human FMR1 gene sequence and associated expression control sequences for controlling expression of the recombinant human FMR1 gene sequence.

In a preferred embodiment, the viral vector includes a portion of a parvovirus genome, such as an AAV genome with rep and cap deleted and/or replaced by the recombinant human FMR1 and its associated expression control sequences. The recombinant human FMR1 gene sequence is typically inserted adjacent to one or two (i.e., is flanked by) AAV TRs or TR elements adequate for viral replication (Xiao et al., 1997, J. Virol. 71(2): 941-948), in place of the nucleic acid encoding viral rep and cap proteins. Other regulatory sequences suitable for use in facilitating tissue-specific expression of the recombinant human FMR1 gene sequence in the target cell may also be included.

One skilled in the art would appreciate that an AAV vector comprising a transgene and lacking virus proteins needed for viral replication (e.g., cap and rep), cannot replicate since such proteins are necessary for virus replication and packaging. Further, AAV is a Dependovirus in that it cannot replicate in a cell without co-infection of the cell by a helper virus. Helper viruses include, typically, adenovirus or herpes simplex virus. Alternatively, as discussed below, the helper functions (E1a, E1b, E2a, E4, and VA RNA) can be provided to a packaging cell including by transfecting the cell with one or more nucleic acids encoding the various helper elements and/or the cell can comprise the nucleic acid encoding the helper protein. For instance, HEK 293 were generated by transforming human cells with adenovirus 5 DNA and now express a number of adenoviral genes, including, but not limited to E1 and E3 (see, e.g., Graham et al., 1977, J. Gen. Virol. 36:59-72). Thus, those helper functions can be provided by the HEK 293 packaging cell without the need of supplying them to the cell by, e.g., a plasmid encoding them.

The viral vector may be any suitable nucleic acid construct, such as a DNA or RNA construct and may be single stranded, double stranded, or duplexed (i.e., self-complementary as described in WO 2001/92551).

One skilled in the art would appreciate that an rAAV vector can further include a “stuffer” or “filler” sequence (filler/stuffer) where the nucleic acid comprising the transgene is less than the approximately 4.1 to 4.9 kb size for optimal packaging of the nucleic acid into the AAV capsid. See, Grieger and Samulski, 2005, J. Virol. 79(15):9933-9944. That is, AAV vectors typically accept inserts of DNA having a defined size range which is generally about 4 kb to about 5.2 kb, or slightly more. Thus, for shorter sequences, inclusion of a filler/stuffer in the insert fragment in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. In particular embodiments of an rAAV vector, a heterologous polynucleotide sequence has a length less than 4.7 Kb and the filler/stuffer polynucleotide sequence has a length that when combined (e.g., inserted into a vector) with the heterologous polynucleotide sequence has a total length between about 3.0-5.5 Kb, or between about 4.0-5.0 Kb, or between about 4.3-4.8 Kb.

An intron can also function as a filler/stuffer polynucleotide sequence in order to achieve a length for AAV vector packaging into a virus particle. Introns and intron fragments that function as a filler/stuffer polynucleotide sequence also can enhance expression. For example, inclusion of an intron element may enhance expression compared with expression in the absence of the intron element (Kurachi et al., 1995, J. Biol. Chem. 270(10):5276-5281). Furthermore, filler/stuffer polynucleotide sequences are well known in the art and include, but are not limited to, those described in WO 2014/144486.

### Viral Capsid

The viral capsid component of the packaged viral vectors may be a parvovirus capsid. AAV Cap and chimeric capsids are preferred. Examples of suitable parvovirus viral capsid components are capsid components from the family Parvoviridae, such as an autonomous parvovirus or a Dependovirus. For example, the viral capsid may be an AAV capsid (e.g., AAV1, AAV2, AAV3, AAV4, AAV5, AAV6, AAV7 AAV8, AAV9, AAV10, AAV11, AAV12, AAV1.1, AAV2.5, AAV6.1, AAV6.3.1, AAV9.45, AAVrh10, AAVrh74, RHM4-1 (SEQ ID NO:5 of WO 2015/013313), AAV2-TT, AAV2-TT-S312N, AAV3B-S312N, AAV-LK03, AAVrh10, AAV-PHP.B, AAV-PHP.eB, AAV-PHP.S, AAV2.GL, AAV2.NN, snake AAV, avian AAV, bovine AAV, canine AAV, equine AAV, ovine AAV, goat AAV, shrimp AAV, and any other AAV now known or later discovered. see, e.g., Fields et al., VIROLOGY, volume 2, chapter 69 (4^{th} ed., Lippincott-Raven Publishers). Capsids may be derived from a number of AAV serotypes disclosed in U.S. Pat. No. 7,906,111; Gao et al., 2004, J. Virol. 78:6381; Moris et al., 2004, Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313, and one skilled in the art would know there are likely other variants not yet identified that perform the same or similar function, or may include components from two or more AAV capsids. A full complement of AAV Cap proteins includes VP1, VP2, and VP3. The ORF comprising nucleotide sequences encoding AAV VP capsid proteins may comprise less than a full complement AAV Cap protein or the full complement of AAV Cap proteins may be provided.

One or more of the AAV Cap proteins may be a chimeric protein, including amino acid sequences of AAV Caps from two or more viruses, preferably two or more AAVs, as described in Rabinowitz et al., U.S. Pat. No. 6,491,907, the entire disclosure of which is incorporated herein by reference. For example, the chimeric virus capsid can include an AAV1 Cap protein or subunit and at least one AAV2 Cap or subunit. The chimeric capsid can, for example, include an AAV capsid with one or more B19 Cap subunits, e.g., an AAV Cap protein or subunit can be replaced by a B19 Cap protein or subunit. For example, in a preferred embodiment, the Vp3 subunit of the AAV capsid can be replaced by the Vp2 subunit of B19.

Another embodiment includes chimeric viral strains synthesized include the combination of AAV backbones from AAV2, AAV3, AAV6, AAV8, etc., with a galactose (Gal) binding footprint from AAV9. Adeno-associated viruses (AAVs) are helper-dependent parvoviruses that exploit heparan sulfate (HS), galactose (Gal), or sialic acids (Sia) as primary receptors for cell surface binding. For instance, AAV serotypes 2 and 3b utilize HS. AAV1, 4, and 5 bind Sia with different linkage specificities, AAV serotype 6, which recognizes both Sia and HS, whereas AAV9 exploits Gal for host cell attachment. Specifically, the galactose (Gal) binding footprint from AAV9 was grafted onto the heparin sulfate-binding AAV serotype 2 and just grafting of orthogonal glycan binding footprints improves transduction efficiency. A new dual glycan-binding strain (AAV2G9) and a chimeric, muscle-tropic strain (AAV2i8G9) were generated by incorporating the Gal binding footprint from AAV9 into the AAV2 VP3 backbone or the chimeric AAV2i8 capsid template using structural alignment and site-directed mutagenesis. In vitro binding and transduction assays confirmed the exploitation of both HS and Gal receptors by AAV2G9 for cell entry. Subsequent in vivo characterization of the kinetics of transgene expression and vector genome biodistribution profiles indicate fast, sustained, and enhanced transgene expression by this rationally engineered chimeric AAV strain. A similar, improved transduction profile was observed with the liver-detargeted, muscle-specific AAV2i8G9 chimera (Shen, et al., 2013, J. Biol. Chem. 288(4):28814-28823). Such new grafting combination is fully described in WO2014/144229 the contents of which are incorporated by reference herein. Additional liver de-targeted AAVs, such as AAV9.45, are described in Pulicherla et al., 2011, Molecular Therapy 19(6):1070-1078, the contents of which are incorporated by reference as if set forth in their entirety herein.

In yet another embodiment the present invention provides for the use of ancestral AAV vectors for use in therapeutic in vivo gene therapy. Specifically, in silico-derived sequences were synthesized de novo and characterized for biological activities. This effort led to the generation of nine functional putative ancestral AAVs and the identification of Anc80, the predicted ancestor of AAV serotypes 1, 2, 8 and 9 (Zinn et al., 2015, Cell Reports 12:1056-1068). Predicting and synthesis of such ancestral sequences in addition to assembling into a virus particle may be accomplished by using the methods described in WO 2015/054653, the contents of which are incorporated by reference herein. Notably, the use of the virus particles assembled from ancestral viral sequences exhibit reduced susceptibility to pre-existing immunity in current day human population than do contemporary viruses or portions thereof.

### Production of Packaged Viral Vector

The invention includes packaging cells, which are encompassed by “host cells,” which may be cultured to produce packaged viral vectors of the invention. The packaging cells of the invention generally include cells with heterologous (1) viral vector function(s), (2) packaging function(s), and (3) helper function(s). Each of these component functions is discussed in the ensuing sections.

Initially, the vectors can be made by several methods known to skilled artisans (see, e.g., WO 2013/063379). A preferred method is described in Grieger, et al. 2015, Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using the triple transfection method (e.g., WO 96/40240), the suspension HEK293 cell line generates greater than 1×10⁵ vector genome containing particles (vg)/cell or greater than 1×10¹⁴ vg/L of cell culture when harvested 48 hours post-transfection. More specifically, triple transfection refers to the fact that the packaging cell is transfected with three plasmids: one plasmid encodes the AAV rep and cap genes, another plasmid encodes various helper functions (e.g., adenovirus or HSV proteins such as E1a, E1b, E2a, E4, and VA RNA, and another plasmid encodes the transgene and its various control elements (e.g., recombinant FMR1 gene and human endogenous promoter, e.g. SEQ ID NO:3).

To achieve the desired yields, several variables are optimized such as selection of a compatible serum-free suspension media that supports both growth and transfection, selection of a transfection reagent, transfection conditions and cell density. A universal purification strategy, based on ion exchange chromatography methods, was also developed that resulted in high purity vector preps of AAV serotypes 1-6, 8, 9 and various chimeric capsids. This user-friendly process can be completed within one week, results in high full to empty particle ratios (>90% full particles), provides post-purification yields (>1×10¹³ vg/L) and purity suitable for clinical applications and is universal with respect to all serotypes and chimeric particles. This scalable manufacturing technology has been utilized to manufacture GMP Phase I clinical AAV vectors for retinal neovascularization (AAV2), Hemophilia B (scAAV8), Giant Axonal Neuropathy (scAAV9) and Retinitis Pigmentosa (AAV2), which have been administered into patients. In addition, a minimum of a 5-fold increase in overall vector production by implementing a perfusion method that entails harvesting rAAV from the culture media at numerous time-points post-transfection.

### Viral Vector Functions

The packaging cells of the invention include viral vector functions, along with packaging and vector functions. The viral vector functions typically include a portion of a parvovirus genome, such as an AAV genome, with rep and cap deleted and replaced by the recombinant human FMR1 sequence and its associated expression control sequences. The viral vector functions include sufficient expression control sequences to result in replication of the viral vector for packaging. Typically, the viral vector includes a portion of a parvovirus genome, such as an AAV genome with rep and cap deleted and replaced by the transgene and its associated expression control sequences. The transgene is typically flanked by two AAV TRs, in place of the deleted viral rep and cap ORFs. Appropriate expression control sequences are included, such as a tissue-specific promoter and other regulatory sequences suitable for use in facilitating tissue-specific expression of the transgene in the target cell. The transgene is typically a nucleic acid sequence that can be expressed to produce a therapeutic polypeptide or a marker polypeptide.

“Duplexed vectors” may interchangeably be referred to herein as “dimeric” or “self-complementary” vectors. The duplexed parvovirus particles may, for example, comprise a parvovirus capsid containing a virion DNA (vDNA). The vDNA is self-complementary so that it may form a hairpin structure upon release from the viral capsid. The duplexed vDNA appears to provide to the host cell a double-stranded DNA that may be expressed (i.e., transcribed and, optionally, translated) by the host cell without the need for second-strand synthesis, as required with conventional parvovirus vectors. Duplexed/self-complementary rAAV vectors are well-known in the art and described, e.g., in WO 2001/92551, WO 2015/006743, and many others.

The viral vector functions may suitably be provided as duplexed vector templates, as described in U.S. Pat. No. 7,465,583 to Samulski et al. (the entire disclosure of which is incorporated herein by reference for its teaching regarding duplexed vectors). Duplexed vectors are dimeric self-complementary (sc) polynucleotides (typically, DNA). The duplexed vector genome preferably contains sufficient packaging sequences for encapsidation within the selected parvovirus capsid (e.g., AAV capsid). Those skilled in the art will appreciate that the duplexed vDNA may not exist in a double-stranded form under all conditions but has the ability to do so under conditions that favor annealing of complementary nucleotide bases. “Duplexed parvovirus particle” encompasses hybrid, chimeric and targeted virus particles. Preferably, the duplexed parvovirus particle has an AAV capsid, which may further be a chimeric or targeted capsid, as described above.

The viral vector functions may suitably be provided as duplexed vector templates, as described in U.S. Pat. No. 7,465,583 to Samulski et al. (the entire disclosure of which is incorporated herein by reference for its teaching regarding duplexed vectors). Duplexed vectors are dimeric self-complementary (sc) polynucleotides (typically, DNA). For example, the DNA of the duplexed vectors can be selected so as to form a double-stranded hairpin structure due to intrastrand base pairing. Both strands of the duplexed DNA vectors may be packaged within a viral capsid. The duplexed vector provides a function comparable to double-stranded DNA virus vectors and can alleviate the need of the target cell to synthesize complementary DNA to the single-stranded genome normally encapsulated by the virus.

The TR(s) (resolvable and non-resolvable) selected for use in the viral vectors are preferably AAV sequences, with serotypes 1, 2, 3, 4, 5 and 6 being preferred. Resolvable AAV TRs need not have a wild-type TR sequence (e.g., a wild-type sequence may be altered by insertion, deletion, truncation or missense mutations), as long as the TR mediates the desired functions, e.g., virus packaging, integration, and/or provirus rescue, and the like. The TRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both TR sequences are from AAV2

The packaging functions include capsid components. The capsid components are preferably from a parvoviral capsid, such as an AAV capsid or a chimeric AAV capsid function. Examples of suitable parvovirus viral capsid components are capsid components from the family Parvoviridae, such as an autonomous parvovirus or a Dependovirus. For example, the capsid components may be selected from AAV capsids, e.g., AAV1, AAV2, AAV3, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAV Hu.26, AAV1.1, AAV2.5, AAV6.1, AAV6.3.1, AAV9.45, AAV218, AAV2G9, AAV218G9, AAV2-TT AAV2-TT-S312N), AAV3B-S312N, and AAV-LK03 (See, U.S. Pat. No. 10,548,947, and other novel capsids as yet unidentified or from non-human primate sources. Capsid components may include components from two or more AAV capsids.

In another embodiment, one or more of the VP capsid proteins is a chimeric protein, comprising amino acid sequences from two or more viruses, preferably two or more AAVs, as described in Rabinowitz et al., U.S. Pat. No. 6,491,907. A chimeric capsid is described herein as having at least one amino acid residue from one serotype combined with another serotype that is sufficient to modify a) viral yield, b) immune response, c) targeting, d) de-targeting, etc.

Further chimeric proteins can be made by instruction set forth in Li, et al., 2008, Mol. Ther. 16(7):1252-1260, the contents of which are incorporated by reference herein. Specifically, a DNA shuffling-based approach was used for developing cell type-specific vectors through directed evolution. Capsid genomes of adeno-associated virus (AAV) serotypes 1-9 were randomly fragmented and reassembled using PCR to generate a chimeric capsid library. A single infectious clone (chimeric-1829) containing genome fragments from AAV1, 2, 8, and 9 was isolated from an integrin minus hamster melanoma cell line previously shown to have low permissiveness to AAV. Molecular modeling studies suggest that AAV2 contributes to surface loops at the icosahedral threefold axis of symmetry, while AAV1 and 9 contribute to two-and five-fold symmetry interactions, respectively. The C-terminal domain (AAV9) was identified as a critical structural determinant of melanoma tropism through rational mutagenesis. Chimeric-1829 utilizes heparan sulfate as a primary receptor and transduces melanoma cells more efficiently than all serotypes. Application of this technology to alternative cell/tissue types using AAV or other viral capsid sequences is likely to yield a new class of biological nanoparticles as vectors for human gene transfer.

The packaged viral vector generally includes the recombinant human FMR1 sequence and expression control sequences flanked by TR elements, referred to herein as the “transgene” or “transgene expression cassette,” sufficient to result in packaging of the vector DNA and subsequent expression of the modified FMR1 sequence in the transduced cell. The viral vector functions may, for example, be supplied to the cell as a component of a plasmid or an amplicon.

The viral vector functions may exist extra chromosomally within the cell line and/or may be integrated into the cell's chromosomal DNA.

Any method of introducing the nucleotide sequence carrying the viral vector functions into a cellular host for replication and packaging may be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

### Packaging Functions

The packaging functions include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions may be supplied together or separately to the packaging cell using a genetic construct such as a plasmid or an amplicon, a Baculovirus, or HSV helper construct. The packaging functions may exist extrachromosomally within the packaging cell but are preferably integrated into the cell's chromosomal DNA. Examples include genes encoding AAV Rep and Cap proteins.

### rAAV Production Systems

Numerous cell culture-based systems are known in the art for production of rAAV particles, any of which can be used to practice a method disclosed herein. The cell culture-based systems include transfection, stable cell line production, and infectious hybrid virus production systems which include Adenovirus-AAV hybrids, herpesvirus-AAV hybrids and baculovirus-AAV hybrids. rAAV production cultures for the production of rAAV virus particles all require; (1) suitable host cells, including, for example, human-derived cell lines such as HeLa, A549, or HEK293 cells and their derivatives (HEK293T cells, HEK293F cells), mammalian cell lines such as Vero, CHO cells or CHO-derived cells, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g. the helper may induce changes to the cellular environment that facilitate such AAV gene expression and replication. In some embodiments, AAV rep and cap genes, helper genes, and rAAV genomes are introduced into cells by transfection of one or more plasmid vectors encoding the AAV rep and cap genes, helper genes, and rAAV genome. In some embodiments, AAV rep and cap genes, helper genes, and rAAV genomes can be introduced into cells by transduction with viral vectors, for example, rHSV vectors encoding the AAV rep and cap genes, helper genes, and rAAV genome. In some embodiments, one or more of AAV rep and cap genes, helper genes, and rAAV genomes are introduced into the cells by transduction with an rHSV vector. In some embodiments, the rHSV vector encodes the AAV rep and cap genes. In some embodiments, the rHSV vector encodes the helper genes. In some embodiments, the rHSV vector encodes the rAAV genome. In some embodiments, the rHSV vector encodes the AAV rep and cap genes. In some embodiments, the rHSV vector encodes the helper genes and the rAAV genome. In some embodiments, the rHSV vector encodes the helper genes and the AAV rep and cap genes.

Any suitable media known in the art may be used for the production of rAAV particles. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety. In some embodiments, the medium comprises Dynamis™ Medium, FreeStyle™ 293 Expression Medium, or Expi293™ Expression Medium from Invitrogen/ThermoFisher. In some embodiments, the medium comprises Dynamis™ Medium. In some embodiments, a method disclosed herein uses a cell culture comprising a serum-free medium, an animal-component free medium, or a chemically defined medium. In some embodiments, the medium is an animal-component free medium. In some embodiments, the medium comprises serum. In some embodiments, the medium comprises fetal bovine serum. In some embodiments, the medium is a glutamine-free medium. In some embodiments, the medium comprises glutamine. In some embodiments, the medium is supplemented with one or more of nutrients, salts, buffering agents, and additives (e.g., antifoam agent). In some embodiments, the medium is supplemented with glutamine. In some embodiments, the medium is supplemented with serum. In some embodiments, the medium is supplemented with fetal bovine serum. In some embodiments, the medium is supplemented with poloxamer, e.g., Kolliphor® P 188 Bio. In some embodiments, a medium is a base medium. In some embodiments, the medium is a feed medium.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, multilayer or multitray tissue culture flasks (or stacks, e.g., hyperstacks), microcarriers, and packed-bed or fluidized-bed bioreactors. rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells and SF-9 cells which can be cultured in a variety of ways including, for example, spinner flasks, stirred tank bioreactors, and disposable systems such as the Wave bag system. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

### Packaging Cell

Any cell or cell line that is known in the art to produce rAAV particles can be used in any one of the methods disclosed herein. In some embodiments, a method of producing rAAV particles or increasing the production of rAAV particles disclosed herein uses HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, a method disclosed herein uses mammalian cells. In some embodiments, a method disclosed herein uses insect cells, e.g., SF-9 cells. In some embodiments, a method disclosed herein uses HEK293 cells. In some embodiments, a method disclosed herein uses HEK293 cells adapted for growth in suspension culture.

In some embodiments, a cell culture disclosed herein is a suspension culture. In some embodiments, a cell culture disclosed herein is a suspension culture comprising HEK293. In some embodiments, a cell culture disclosed herein is a suspension culture comprising HEK293 cells adapted for growth in suspension culture. In some embodiments, a cell culture disclosed herein comprises a serum-free medium, an animal-component free medium, or a chemically defined medium. In some embodiments, a cell culture disclosed herein comprises a serum-free medium. In some embodiments, suspension-adapted cells are cultured in a shaker flask, a spinner flask, a cell bag, or a bioreactor.

In some embodiments, a cell culture disclosed herein comprises cells attached to a substrate (e.g., microcarriers) that are themselves in suspension in a medium. In some embodiments, the cells are HEK293 cells.

In some embodiments, a cell culture disclosed herein is an adherent culture. In some embodiments, a cell culture disclosed herein is an adherent culture comprising HEK293. In some embodiments, a cell culture disclosed herein comprises a serum-free medium, an animal-component free medium, or a chemically defined medium. In some embodiments, a cell culture disclosed herein comprises a serum-free medium.

In some embodiments, a cell culture disclosed herein comprises a high-density cell culture. In some embodiments, the culture has a total cell density of between about 1×10E+06 cells/ml and about 30×10E+06 cells/ml. In some embodiments, more than about 50% of the cells are viable cells. In some embodiments, the cells are Hela cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

Cell lines for use as packaging cells include insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et al., Baculovirus Expression Vectors: A Laboratory Manual, Oxford Univ. Press (1994); Samulski et al., 1989, J. Virol. 63:3822-3828; Kajigaya et al., 1991, Proc. Nat'l. Acad. Sci. USA 88:4646-4650; Ruffing et al., 1992, J. Virol. 66:6922-6930; Kimbauer et al., 1996, Virol. 219:37-44; Zhao et al., 2000, Virol. 272:382-393; and Samulski et al., U.S. Pat. No. 6,204,059.

For example, virus capsids utilized in embodiments described herein can be produced using any method known in the art, e.g., by expression from a baculovirus (Brown et al., (1994) Virology 198:477-488). As a further alternative, the virus vectors of the invention can be produced in insect cells using baculovirus vectors to deliver the rep/cap genes and rAAV template as described, for example, by Urabe et al., 2002, Human Gene Therapy 13:1935-1943.

In another aspect, provided herein are methods of rAAV production in insect cells wherein a baculovirus packaging system or vectors may be constructed to carry the AAV Rep and Cap coding region by engineering these genes into the polyhedrin coding region of a baculovirus vector and producing viral recombinants by transfection into a host cell. Notably when using Baculovirus production for AAV, preferably the AAV DNA vector product is a self-complementary AAV like molecule without using mutation to the AAV ITR. This appears to be a by-product of inefficient AAV rep nicking in insect cells which results in a self-complementary DNA molecule by virtue of lack of functional Rep enzyme activity. The host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids.

During production, the packaging cells generally include one or more viral vector functions along with helper functions and packaging functions sufficient to result in replication and packaging of the viral vector. These various functions may be supplied together or separately to the packaging cell using a genetic construct such as a plasmid or an amplicon, and they may exist extrachromosomally within the cell line or integrated into the cell's chromosomes.

The cells may be supplied with any one or more of the stated functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA.

### rAAV Purification

The rAAV particles produced can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, sterile filtration, or any combination(s) thereof. In some embodiments, downstream processing includes at least 2, at least 3, at least 4, at least 5 or at least 6 of: harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography. In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation.

In some embodiments, a method of isolating rAAV particles comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, a method of isolating rAAV particles comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In some embodiments, clarification of the harvested cell culture comprises sterile filtration.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. rAAV production cultures can further contain product-related impurities, for example, inactive vector forms, empty viral capsids, aggregated viral particles or capsids, mis-folded viral capsids, degraded viral particle.

In some embodiments, the rAAV production culture harvest is clarified to remove host cell debris. In some embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In some embodiments, clarification of the production culture harvest does not included centrifugation.

In some embodiments, harvested cell culture is clarified using filtration. In some embodiments, clarification of the harvested cell culture comprises depth filtration. In some embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In some embodiments, the filter train comprises 2 depth filtration media and a sterile filtration media. In some embodiments, the depth filter media is a porous depth filter. In some embodiments, the filter train comprises Clarisolve® 20MS, Millistak+® C0HC, and a sterilizing grade filter media. In some embodiments, the filter train comprises Clarisolve® 20MS, Millistak+® C0HC, and Sartopore® 2 XLG 0.2 pm. In some embodiments, the harvested cell culture is pretreated before contacting it with the depth filter. In some embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul et al, Human Gene Therapy 4:609-615 (1993). TFF concentration of the clarified feed enables a technically manageable volume of clarified feed to be subjected to chromatography and allows for more reasonable sizing of columns without the need for lengthy recirculation times. In some embodiments, the clarified feed is concentrated between at least two-fold and at least ten-fold. In some embodiments, the clarified feed is concentrated between at least ten-fold and at least twenty-fold. In some embodiments, the clarified feed is concentrated between at least twenty-fold and at least fifty-fold. In some embodiments, the clarified feed is concentrated about twenty-fold. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In some embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed disclosed herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™, POROS™ CaptureSelect™ AAVX affinity resin, POROS™ CaptureSelect™ AAV9 affinity resin, and POROS™ CaptureSelect™ AAV8 affinity resin. In some embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, Unosphere Q (Biorad, Hercules, Calif.), and N-charged amino or imino resins such as e.g., POROS 50 PI, or any DEAE, TMAE, tertiary or quaternary amine, or PEI-based resins known in the art (U.S. Pat. No. 6,989,264; Brument et al., Mol. Therapy 6(5):678-686 (2002); Gao et al., Hum. Gene Therapy 11:2079-2091 (2000)). In some embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers. In some embodiments, the monolith anion exchange chromatography media is selected from the group consisting of CIMmultus™ QA-1 Advanced Composite Column (Quaternary amine), CIMmultus™ DEAE-1 Advanced Composite Column (Diethylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In some embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In additional embodiments the disclosure provides compositions comprising isolated rAAV particles produced according to a method disclosed herein. In some embodiment, the composition is a pharmaceutical composition comprising a pharmaceutically acceptable carrier.

As used herein the term “pharmaceutically acceptable means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al, Drug Delivery Systems (1980), R. L. Juliano, ed., Oxford, N.Y., pp. 253-315).

As described herein, the recombinant polynucleotide constructs and rAAVs can be used as a gene therapy to treat Fragile X or related disorder related to a lack of FMRP. Methods of treatment include injecting any of the rAAV's described herein into a subject in need thereof. One skilled in the art would understand the quantities needed to treat the subject, as it would depend on multiple factors including size, age, and gender of the subject.

### Methods of Treatment

In another aspect, methods of treatment are presented. The method comprises administering an effective amount of the pharmaceutical composition comprising any of the desired constructs or rAAV virions described above to a patient in need thereof.

In some embodiments, the effective amount is at least 1×10⁸ viral genomes per dose. In some embodiments, the effective amount is at least 5×10⁸ viral genomes/dose, 7.5×10⁸ viral genomes/dose, at least 1×10⁹ viral genomes/dose, at least 2.5×10⁹ viral genomes/dose, at least 5×10⁹ viral genomes/dose.

In some embodiments, the effective amount is at least 1×10¹¹ viral genomes/kg patient weight, at least 5×10¹¹ viral genomes/kg, at least 1×10¹² viral genomes/kg, at least 5×10¹² viral genomes/kg, at least 1×10¹³ viral genomes/kg, at least 1×10¹⁴ viral genomes/kg, or at least 5×10¹⁴. In some embodiments, the rAAV is dosed based upon brain weight rather than by bodyweight. In some embodiments, the rAAV dose is considered a low dose and is particularly beneficial for a CNS indication.

In some embodiments, the rAAV is administered intravenously. In some embodiments, the rAAV is administered intrathecally. In some embodiments, the rAAV is administered by intracerebral ventricular injection. In some embodiments, the rAAV is administered by intracisternal magna administration. In some embodiments, the rAAV is administered by intravitreal injection.

In various embodiments a method of treating a FXS and related disorders in a subject is disclosed, wherein the method comprises administering to the subject an effective amount of any of the polynucleotide constructs described herein, or the vectors, or the rAAV comprising the vectors, or the virion, or any pharmaceutical composition comprising any of these elements, as described herein.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1 Optimizing the Recombinant FMR1 Construct

FIG. 1 illustrates (FIG. 1A) the FMR1 construct driven by expression from the constitutive CBh promoter (SEQ ID NO:7), and (FIG. 1B) the low levels of transgenic FMRP expression observed by immunohistochemistry analysis 70 days after injection into P0-2 wild-type mice by ICV delivery. AAV9-CBh-hFMR1-Myc-bGHpA was delivered at two different doses, and levels of FMRP compared to vehicle-treated wild-type mice.

FIGS. 2-3 illustrate an exemplary FMR1 construct design incorporating endogenous promoter and 3′UTR fragments, and plasmid. The FMR1 construct containing hFMP1050-hFMR1-Myc-FMpA1 has a sequence of SEQ ID NO:2. The plasmid containing the hFMP1050-hFMR1-Myc-FMpA1 construct has a sequence of SEQ ID NO: 1.

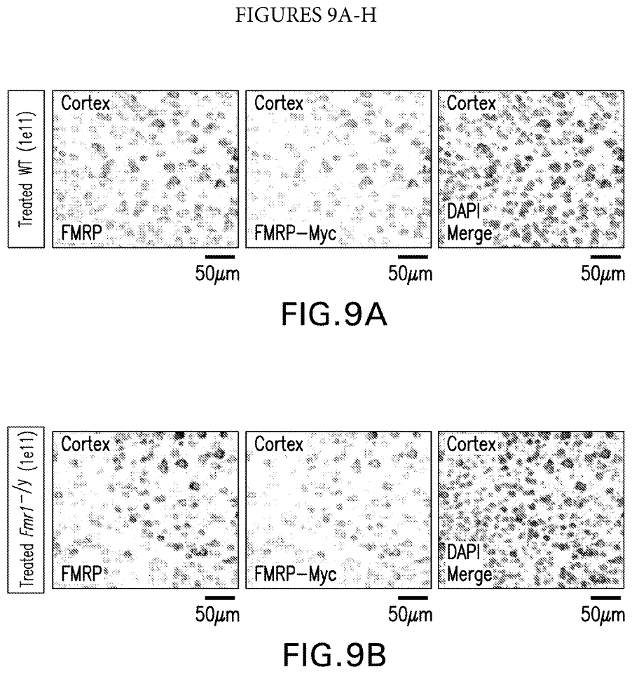

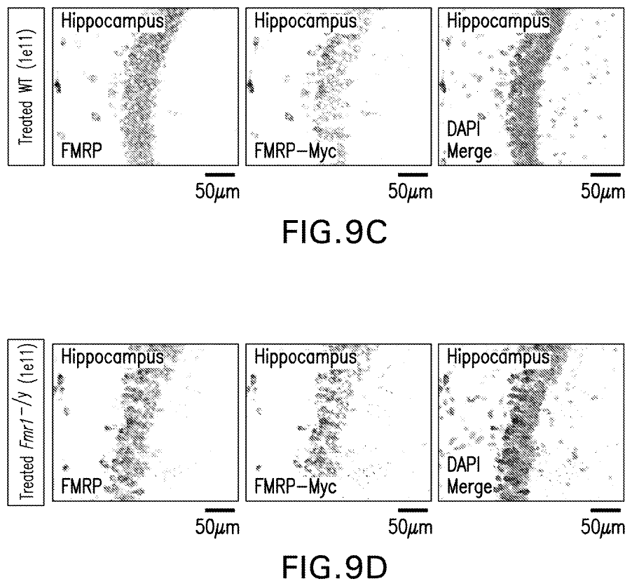

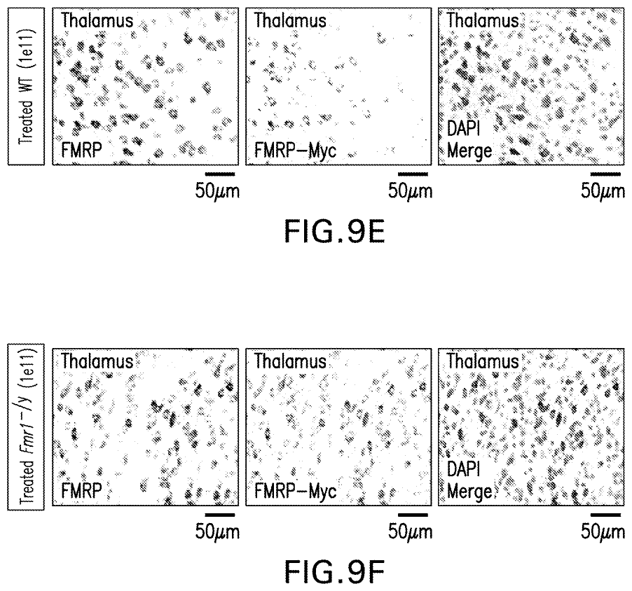

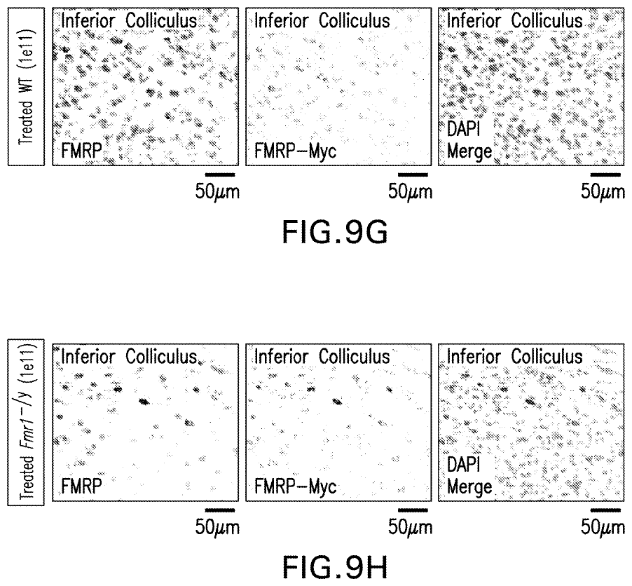

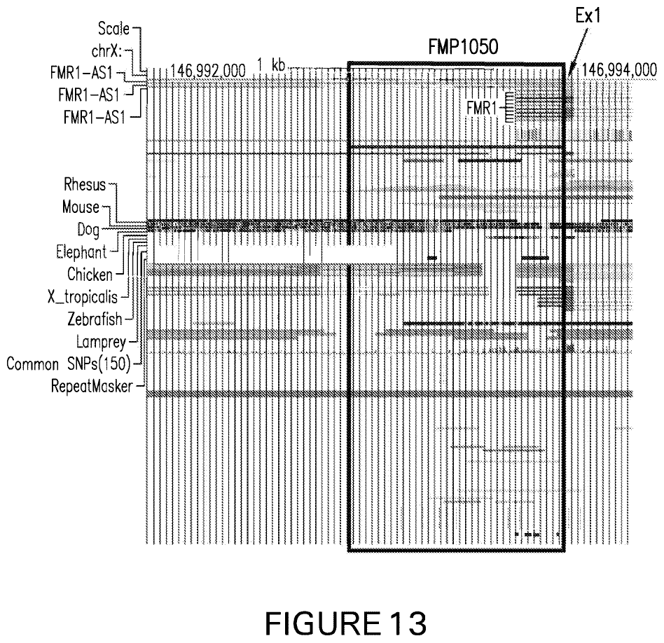

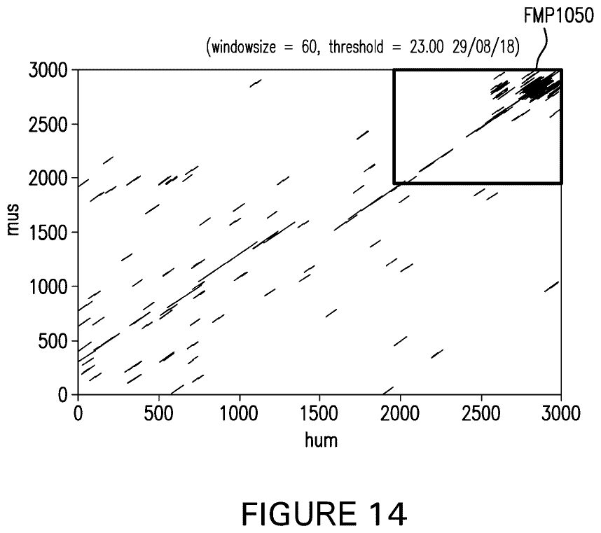

The 1050 bp fragment of the human FMR1 promoter and 5′UTR, immediately upstream of the ATG codon in exon 1, was predicted to contain most of the key genetic regulatory elements needed for near-endogenous regulation of FMR1 transgene expression. This 1050 bp fragment (SEQ ID NO:3) included: CpG islands in the core promoter region, Transcription Factor binding sites, cis regulatory elements identified by ENCODE, H3K27Ac, and regulatory elements from the Open Regulatory Annotation database (ORegAnno) (FIG. 13). H3K27Ac marks indicating epigenetic modification of the DNA packaging protein histone H3, and therefore defined as an active enhancer mark. Regulatory elements from ORegAnno represent the predominant DNase clusters in this genomic region, and key, conserved Transcription Factor binding sites. Such elements are not found in the genomic region upstream of the 1050 bp promoter fragment. In addition, the 1050 bp promoter fragment (SEQ ID NO: 3) is well conserved between human and mouse, suggesting that this region harbors important, conserved sequences (FIG. 14). Therefore, this 1050 bp fragment in the FMR1 promoter region (SEQ ID NO: 3) is capable of recapitulating endogenous FMR1 promoter in driving expression of FMR1 in the brain. FIG. 9A-H shows FMR1 expression detected in various regions of the brain (cortex, hippocampus, thalamus, inferior colliculus) under the control of hFMP1050 promoter fragment (hFMP1050-hFMR1-Myc-FMpA1) (SEQ ID NO:2).

### Example 2 Assessment of Behavioral Phenotypes and Immunoblot Data for Localization of FMRP in Brain Tissue Samples

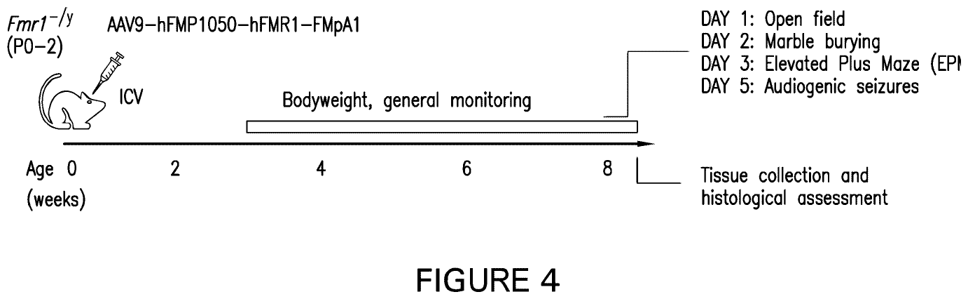

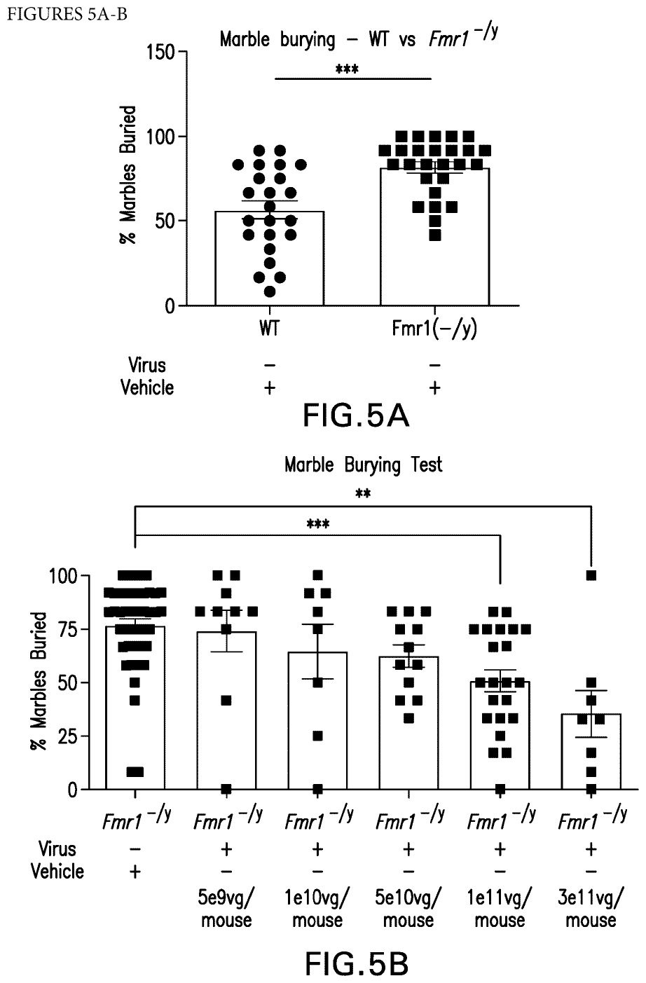

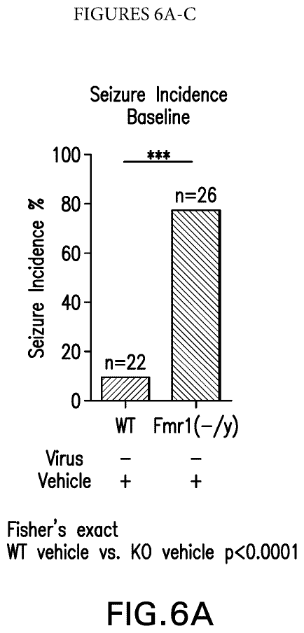

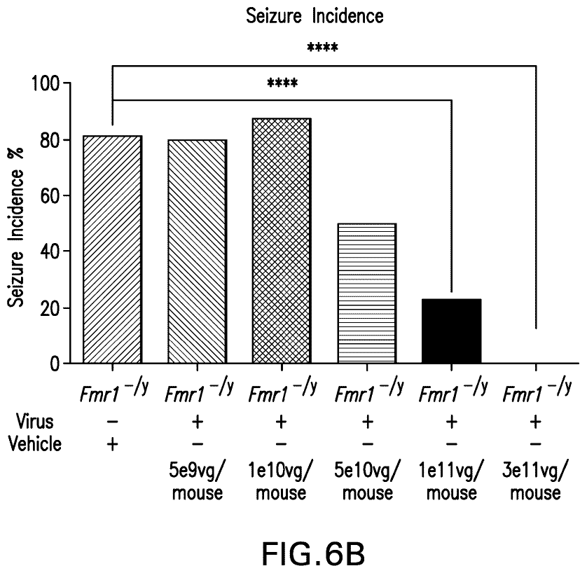

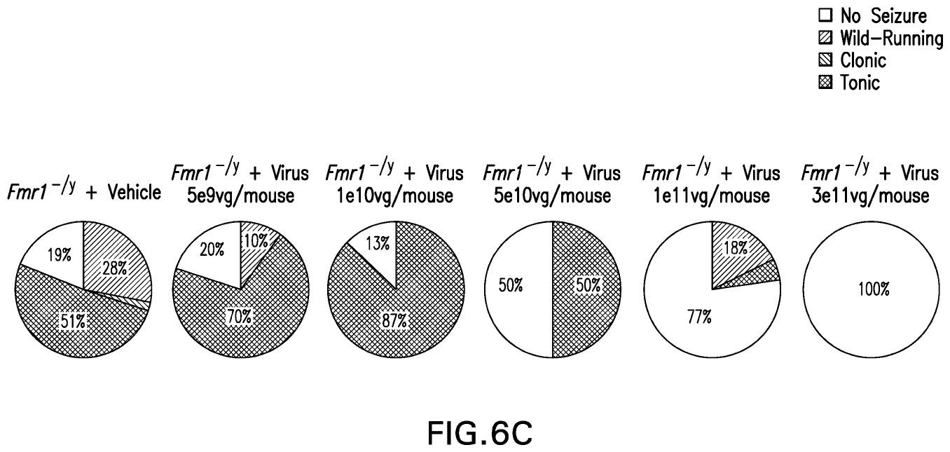

FIG. 4 shows the experimental design and a schematic of the various phenotyping analysis utilized to test Fmr1^{-/y} mice following dosing with AAV9-hFMP1050-hFMR1-Myc-FMpA1. FMR1 expression in the Fmr1^{-/y} mice was driven under the control of hFMP1050 promoter fragment (SEQ ID NO:2). Individual mice were injected administered with AAV9-hFMP1050-hFMR1-Myc-FMpA1 via intracerebroventricular injection (ICV) at week 0. Mice were left to develop for 8 weeks. Beginning on week 3, mice were monitored for general bodyweight changes. At week 8, mice were assessed by varies behavioral assays: open filed assay on Day 1; marble burying assay on Day 2; Elevated Plus Maze (EPM) assay on Day 3; audiogenic seizures assay on Day 5. Tissues from individual mouse were collected for histological assessment. The results are shown in FIGS. 5-6.

FIGS. 5A-B show an observable therapeutic treatment effect on marble burying in Fmr1^{-/y} mice receiving different doses of AAV9-hFMP1050-hFMR1-Myc-FMpA1. As shown in FIG. 5A, Fmr1^{-/y} mutant mice exhibited significantly higher (p<0.0001, Mann-Whitney test) tendency of marble burying behavior compared to wild type (WT) mice. Expression of hFMR1 in the mutant mice significantly reduced marble burying behavior in a dose-dependent manner (FIG. 5B). The data show that a dose of 1e11 vg/mouse (p value) of AAV9-hFMP1050-hFMR1-Myc-FMpA1 was sufficient to significantly reduce marble behavior (p=0.0004, Kruskal-Wallis test with Dunn's post-hoc analysis), and the therapeutic effect was further improved at a higher dosage (3e11 vg/mouse) (p=0.0024, Kruskal-Wallis test with Dunn's post-hoc analysis).

FIG. 6A-C illustrates (FIG. 6A) the strong genotype effect observed in the Fmr1^{-/y} mouse model for audiogenic seizures; (FIG. 6B-C) the therapeutic effect of treatment with AAV9-hFMP1050-hFMR1-Myc-FMpA1 (Virus). FIG. 6B shows treatment with AAV9-hFMP1050-hFMR1-Myc-FMpA1 in Fmr1^{-/y} mutant mice significantly reduced seizure incidence at a dose of 5e10 vg/mouse (p=0.0554, Fisher's exact test). The therapeutic effect was more pronounced at higher doses of 1e11 or 3e11 vg/mouse (p<0.0001, Fisher's exact test). FIG. 6C shows at the highest dose (3e11 vg/mouse) of AAV9-hFMP1050-hFMR1-Myc-FMpA1, clonic and tonic seizure absent, compared to 81% in control, vehicle-treated animals.

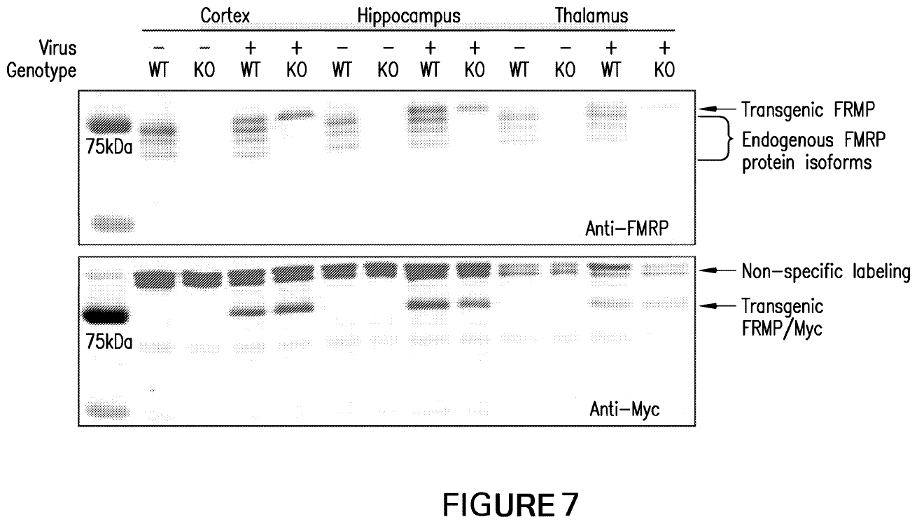

FIG. 7 shows immunoblot data from cortex, hippocampus and thalamus of wild-type (WT) and Fmr1^{-/y} (KO) mice dosed with vehicle or 5E10 vg/mouse of AAV9-AAV9-hFMP1050-hFMR1-Myc-FMpA1. Transgenic FMRP was detected in cortex and hippocampus after administration of 5E10 vg/mouse of AAV9-AAV9-hFMP1050-hFMR1-Myc-FMpA1. Myc expression was used as a positive control.

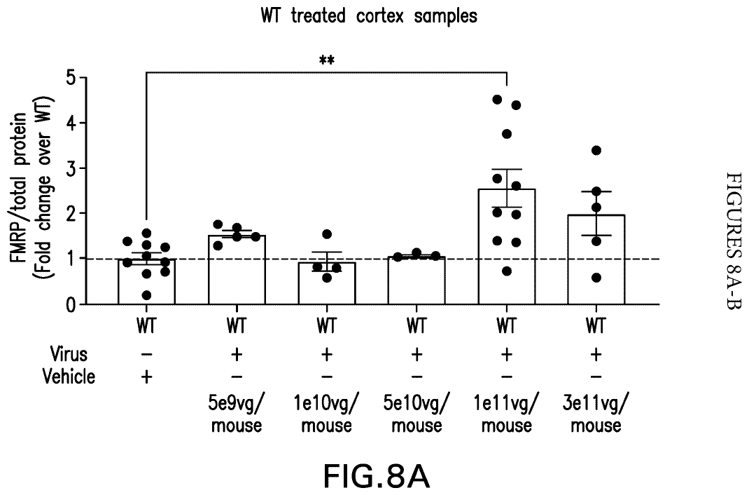

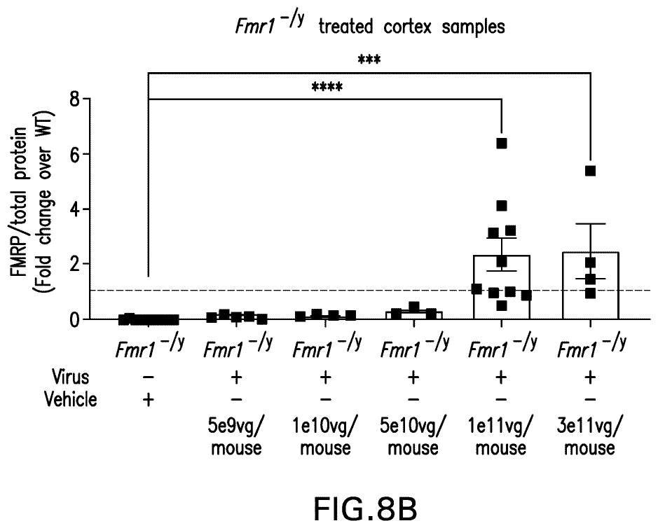

FIGS. 8A-B show quantification of FMRP expression levels in the cortex of wild-type (WT) and Fmr1^{-/y} (KO) mice treated with increasing doses of AAV9-hFMP1050-hFMR1-Myc-FMpA1. At a dose of 1e11 vg/mouse, FMRP expression was detected in the cortex of WT treated mice (FIG. 8A) and in Fmr1^{-/y} (KO) treated mice (FIG. 8B).

FIGS. 9A-H show immunohistochemistry analysis of transgenic FMRP expression in different brain areas in mice 70 days after injection with AAV9-hFMP1050-hFMR1-myc-FMpA1. Wild-type (WT) and Fmr1^{-/y} mice were treated at P0-2 by ICV delivery of 1E11 vg/mouse. FMRP expression was detected in cortex (FIG. 9B) and hippocampus (FIG. 9D). The imaging data is consistent with the detection of immunoblot data of FMRP expression in FIG. 7.

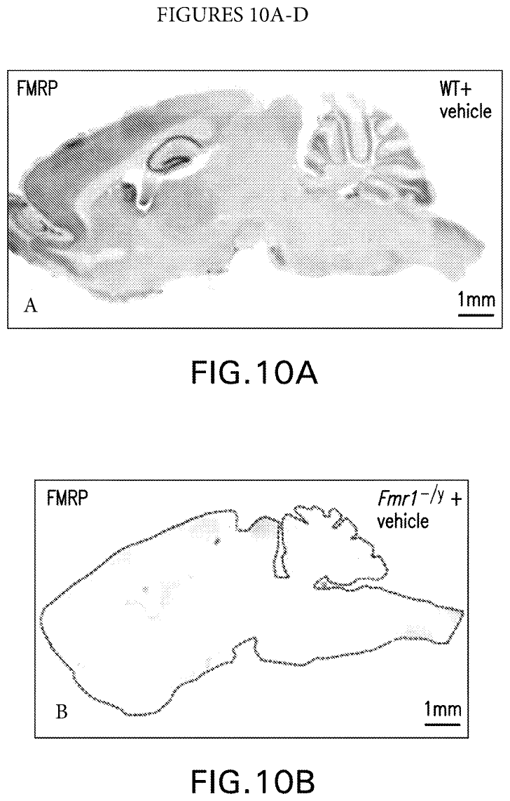

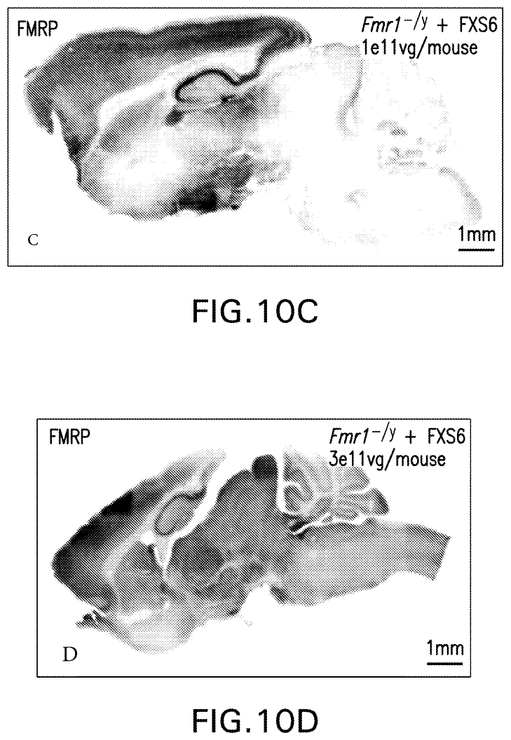

FIGS. 10A-D show increased FMRP expression after delivery of AAV9-hFMP1050-hFMR1-Myc-FMpA1 containing SEQ ID NO:2 to Fmr1^{-/y} mice. Representative tiled sagittal whole brain micrographs, immunolabelled against FMRP (green), from (FIG. 10A) a vehicle-treated WT mouse, (FIG. 10B) a vehicle-treated Fmr1^{-/y} mouse, (FIGS. 10 C-D) an Fmr1^{-/y} mouse treated with AAV9-hFMP1050-hFMR1-Myc-FMpA1 at a dose of 1E11 vg/mouse (FIG. 10C) or 3E11 vg/mouse (FIG. 10D). Scale bars represent 1 mm.

### Example 3: Further Modified FMR1 Constructs and Elements

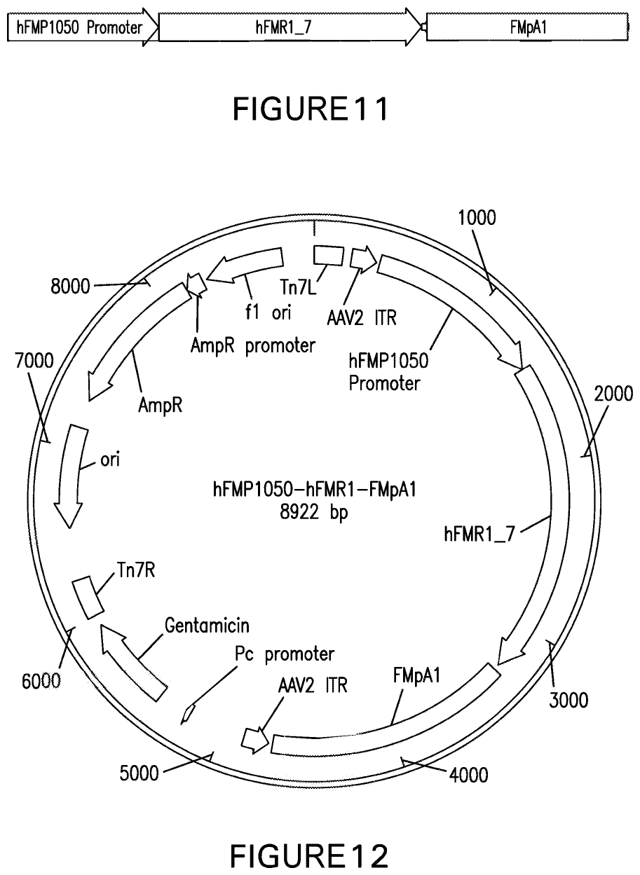

FIG. 11 illustrates the FMR1 construct design incorporating a 1050 bp fragment of the endogenous FMR1 promoter (hFMP1050), a human wild-type FMR1 sequence coding for isoform 7 (hFMR1), and a fragment of the endogenous 3′UTR of human FMR1 (FMpA1). This construct lacks a Myc tag and is an illustrative therapeutic FMR1 construct. FIG. 12 illustrates a plasmid FMR1 construct containing the hMP1050-FMR1-FMpA1 construct flanked by wild-type AAV2 inverted terminal repeats (ITRs).

A modified FMR1 gene therapy construct was designed to include an additional regulatory element, identified in the 3′UTR of human FMR1. The new 3′UTR fragment, designated FMpA2 (SEQ ID NO:13), is an extended version of FMpA1, harboring an additional 181 bp of human 3′UTR sequence that contains a putative CNS enhancer element.

To generate a construct that could still be packaged in single-stranded AAV, the 1050 bp fragment of the FMR1 promoter was truncated. Most key transcription factor binding sites and CNS-associated DNAse sites that showed enhancer-like activity were present in a 734 bp fragment of the FMR1 promoter (SEQ ID NO: 12; also incorporating the 5′UTR, immediately upstream of the FMR1 coding sequence). FIG. 13 shows the FMR1 promoter region in the UCSC Genome Browser. The 1050 bp fragment contains most of the key genetic regulatory elements in this genomic region needed for endogenous expression and regulation of FMR1. FIG. 14 shows a dotplot, generated by EMBOSS Dotmatcher, that compares the sequences of human (hum) and mouse (mus) in the genomic region upstream of the FMR1 coding region, incorporating the 1050 bp fragment of the promoter.

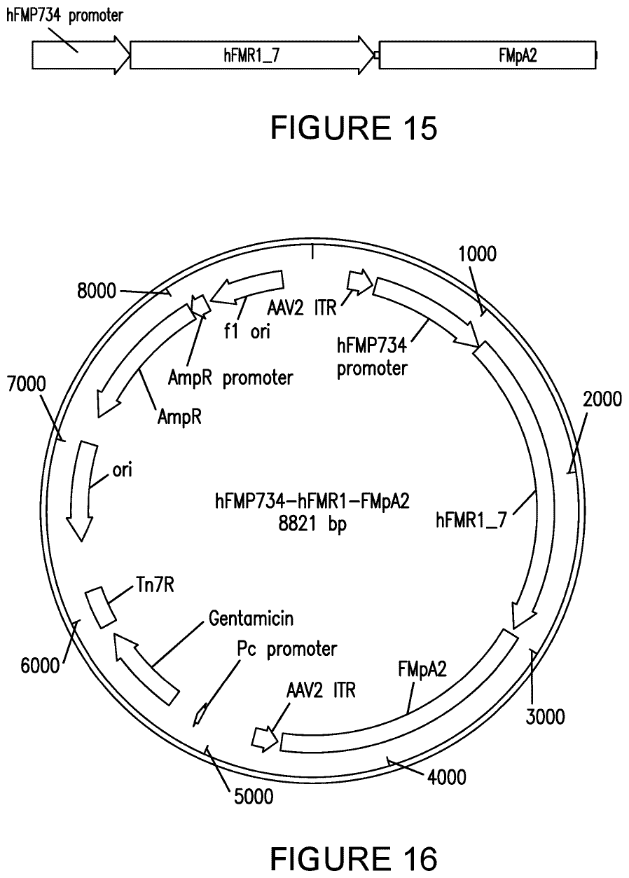

The selected truncated FMR1 promoter region was hMP734 promoter fragment, which was combined with the FMpA2 3′UTR sequence to generate the hFMP734-hFMR1-FMpA2 construct (SEQ ID NO:11) to drive and regulate expression of FMRP. As shown in FIG. 15, the hFMP734-hFMR1-FMpA2 construct (SEQ ID NO:11) incorporates a 734 bp fragment of the endogenous FMR1 promoter (hFMP734), a human wild-type FMR1 sequence coding for isoform 7 (hFMR1), and a fragment of the endogenous 3′UTR of human FMR1 (FMpA2). FIG. 16 illustrates an hMP734-FMR1-FMpA2 plasmid (SEQ ID NO:10) containing the hMP734-FMR1-FMpA2 construct flanked by wild-type AAV2 inverted terminal repeats (ITRs).

### Example 4: Comparing the hFMP1050-hFMR1-FMpA1 and hFMP734-hFMR1-FMpA2 Constructs

To compare FMRP expression from these two constructs, the plasmid (SEQ ID NO: 8) expressing FMRP from a hFMP1050-hFMR1-FMpA1 construct (FIG. 11, SEQ ID NO: 9), and the plasmid (SEQ ID NO:10) expressing FMRP from a hFMP734-hFMR1-FMpA2 construct (FIG. 15, SEQ ID NO: 11) were transfected into HEK293A cells and then analyzed for FMRP expression levels by immunoblot.

For transfections, HEK293A cells maintained in supplemented Dulbecco's modified Eagle's medium (DMEM) were cultured to 80-90% confluency before the day of transfection, trypsinized (TrypLE™ Express, Life Technologies, Carlsbad, CA) and seeded into 24 well plates coated with poly-L-ornithine and fibronectin. Cells were transfected using Lipofectamine 2000 (Life Technologies), following the manufacturer's protocol, with either 2000 ng, 1000 ng, 500 ng, 250 ng or 125 ng plasmid DNA mixed with Opti-MEM (ThermoFisher Scientific) per well. At 48 h post-transfection, cells were prepared for immunoblot by removing the media, and transferring cells to NE1 buffer (20 mM HEPES, 10 mM KCl, 1 mM MgCl2, 0.1% Triton X-100, 20% glycerol, 0.5 mM DTT, Pierce Protease Inhibitor Tablet, EDTA-free [A32965, ThermoFisher Scientific]). Genomic DNA was removed with a benzonase digest (E1014, Merck, Rockville, MD, USA) for 15 min at room temperature. Protein concentration was quantified using DC BioRad II (5000112, BioRad, Hercules, CA, USA) in a microtiter plate using a FLUOstar Omega plate reader (BMG Labtech, Cary, NC, USA). Samples were diluted in one-third volume 4× Laemmli buffer (0.5M Tris-HCl, 8% SDS, 0.01% bromophenol blue, 40% glycerol, 10% beta-mercaptoethanol) and denatured at 95° C. for 10 min before being storing at −80° C. Protein samples (20 μg/lane) were resolved and transferred to nitrocellulose membranes (BioRad). Total protein was measured using Revert 700 Total Protein Stain (926-11010, LI-COR, Lincoln, NB, USA), and membranes were blocked in Intercept (TBS) Blocking buffer (927-60001, LI-COR) for 1 h at room temperature. Rabbit anti-FMRP primary antibody (ab17722; Abcam; 1:1,000) was incubated at 4° C. overnight. Secondary antibodies (IRDye 800CW donkey anti-rabbit IgG secondary antibody (926-32213; LI-COR); 1:10,000) were incubated for 1 h at room temperature before data captured on a LI-COR Odyssey Classic. Data were analyzed using Image Studio Lite (LI-COR) and Microsoft Excel.

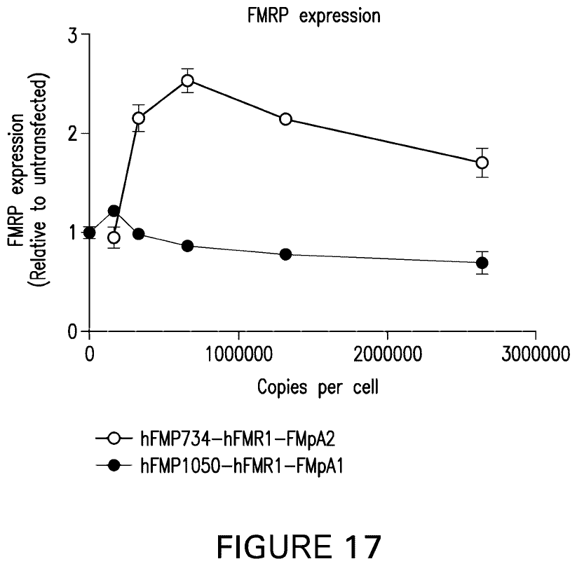

FIG. 17 shows immunoblot data of FMRP expression from plasmids containing hFMP1050-hFMR1-FMpA1 (SEQ ID NO:8) or hFMP734-hFMR1-FMpA2 (SEQ ID NO:10). FMRP expression from hFMP1050-hFMR1-FMpA1 (SEQ ID NO:9) was not detected at significant levels above background FMRP expression (endogenous levels of FMRP in HEK293A cells) at any of the DNA input levels tested. FMRP expression from hFMP734-hFMR1-FMpA2 (SEQ ID NO:11) was detected at significant levels above background FMRP expression when input levels of DNA were 250 ng per 24-well and above. An important test of expression for therapeutic use is illustrated by the healthy expression observed from the hFMP1050-hFMRP-FMpA1 construct in vivo (See, FIGS. 4-11). However, in vitro expression in cells lines such as HEK293A also predicts expression from therapeutic transgene cassettes. FMRP expression from the more minimal hFMP734 promoter (from the hMFP734-hFMR1-FMpA2 construct) results in more robust expression relative to the larger hFMP1050 promoter (from the hFMP1050-hFMRP-FMpA1 construct) in vitro (FIG. 17). This stronger expression afforded by the more minimal promoter fragment (FIG. 17) is unexpected and provides an alternative, more potent and robust expression cassette sequence.

### Sequence Listing and Features

| SEQ ID NO | Construct |
|---|---|
| SEQ ID NO: 1 | hFMP1050-hFMR1-Myc-FMpA1 plasmid |
| SEQ ID NO: 2 | hFMP1050-hFMR1-Myc-FMpA1 |
| SEQ ID NO: 3 | hFMP1050, human core promoter |
| SEQ ID NO: 4 | Wild-type human FMR1 isoform 7 |
| SEQ ID NO: 5 | Endogenous human FMR1 3′UTR and |
| | polyA (FMpA1) |
| SEQ ID NO: 6 | Human FMRP |
| SEQ ID NO: 7 | CBh-hFMR1-bGHpA |
| SEQ ID NO: 8 | hFMP1050-hFMR1-FMpA1 plasmid |
| SEQ ID NO: 9 | hFMP1050-hFMR1-FMpA1 |
| SEQ ID NO: 10 | hFMP734-hFMR1-FMpA2 plasmid |
| SEQ ID NO: 11 | hFMP734-hFMR1-FMpA2 |
| SEQ ID NO: 12 | hFMP734, human core promoter |
| SEQ ID NO: 13 | Endogenous human FMR1 3′UTR and |
| | polyA (FMpA2) |

Sequence of the plasmid hFMP1050-hFMR1-Myc-FMpA1
•hFMP1050 promoter sequence in bold
•hFMR1 GOI sequence in bold and underlined
•Myc tag sequence in bold, italics and underlined
•FMpA1 sequence underlined
>hFMP1050-hFMR1-Myc-FMpA1 (SEQ ID NO: 1)
ACGCCAGCAACGCGGCCTTTTTACGGTTCCTGGCCTTTTGCTGGCCTTTTGCTCACATGTCCTGCAGG
CAGCTGCGCGCTCGCTCGCTCACTGAGGCCGCCCGGGCGTCGGGCGACCTTTGGTCGCCCGGCCTCAG
TGAGCGAGCGAGCGCGCAGAGAGGGAGTGGCCAACTCCATCACTAGGGGTTCCTGCGGCCTCTAGAAA
GCTTCTCTGGCACAGGGTGCACATACAGTAGGGGCAGAAATGAACCTCAAGTGCTTAACACAATTTTT
AAAAAATATATAGTCAAGTGAAAGTATGAAAATGAGTTGAGGAAAGGCGAGTACGTGGGTCAAAGCTG
GGTCTGAGGAAAGGCTCACATTTTGAGATCCCGACTCAATCCATGTCCCTTAAAGGGCACAGGGTGTC
TCCACAGGGCCGCCCAAAATCTGGTGAGAGAGGGCGTAGACGCCTCACCTTCTGCCTCTACGGGTCAC
AAAAGCCTGGGTCACCCTGGTTGCCACTGTTCCTAGTTCAAAGTCTTCTTCTGTCTAATCCTTCACCC
CTATTCTCGCCTTCCACTCCACCTCCCGCTCAGTCAGACTGCGCTACTTTGAACCGGACCAAACCAAA
CCAAACCAAACCAAACCAAACCAGACCAGACACCCCCTCCCGCGGAATCCCAGAGAGGCCGAACTGGG
ATAACCGGATGCATTTGATTTCCCACGCCACTGAGTGCACCTCTGCAGAAATGGGCGTTCTGGCCCTC
GCGAGGCAGTGCGACCTGTCACCGCCCTTCAGCCTTCCCGCCCTCCACCAAGCCCGCGCACGCCCGGC
CCGCGCGTCTGTCTTTCGACCCGGCACCCCGGCCGGTTCCCAGCAGCGCGCATGCGCGCGCTCCCAGG
CCACTTGAAGAGAGAGGGCGGGGCCGAGGGGCTGAGCCCGCGGGGGGAGGGAACAGCGTTGATCACGT
GACGTGGTTTCAGTGTTTACACCCGCAGCGGGCCGGGGGTTCGGCCTCAGTCAGGCGCTCAGCTCCGT
TTCGGTTTCACTTCCGGTGGAGGGCCGCCTCTGAGCGGGCGGCGGGCCGACGGCGAGCGCGGGCGGCG
GCGGTGACGGAGGCGCCGCTGCCAGGGGGCGTGCGGCAGCGCGGCGGCGGCGGCGGCGGCGGCGGCGG
CGGAGGCGGCGGCGGCGGCGGCGGCGGCGGCGGCTGGGCCTCGAGCGCCCGCAGCCCACCTCTCGGGG
GCGGGCTCCCGGCGCTAGCAGGGCTGAAGAGAAGATGGAGGAGCTGGTGGTGGAAGTGCGGGGCTCCA
ATGGCGCTTTCTACAAGGCATTTGTAAAGGATGTTCATGAAGATTCAATAACAGTTGCATTTGAAAAC
AACTGGCAGCCTGATAGGCAGATTCCATTTCATGATGTCAGATTCCCACCTCCTGTAGGTTATAATAA
AGATATAAATGAAAGTGATGAAGTTGAGGTGTATTCCAGAGCAAATGAAAAAGAGCCTTGCTGTTGGT
GGTTAGCTAAAGTGAGGATGATAAAGGGTGAGTTTTATGTGATAGAATATGCAGCATGTGATGCAACT
TACAATGAAATTGTCACAATTGAACGTCTAAGATCTGTTAATCCCAACAAACCTGCCACAAAAGATAC
TTTCCATAAGATCAAGCTGGATGTGCCAGAAGACTTACGGCAAATGTGTGCCAAAGAGGCGGCACATA
AGGATTTTAAAAAGGCAGTTGGTGCCTTTTCTGTAACTTATGATCCAGAAAATTATCAGCTTGTCATT
TTGTCCATCAATGAAGTCACCTCAAAGCGAGCACATATGCTGATTGACATGCACTTTCGGAGTCTGCG
CACTAAGTTGTCTCTGATAATGAGAAATGAAGAAGCTAGTAAGCAGCTGGAGAGTTCAAGGCAGCTTG
CCTCGAGATTTCATGAACAGTTTATCGTAAGAGAAGATCTGATGGGTCTAGCTATTGGTACTCATGGT
GCTAATATTCAGCAAGCTAGAAAAGTACCTGGGGTCACTGCTATTGATCTAGATGAAGATACCTGCAC
ATTTCATATTTATGGAGAGGATCAGGATGCAGTGAAAAAAGCTAGAAGTTTTCTCGAATTTGCTGAAG
ATGTAATACAAGTTCCAAGGAACTTAGTAGGCAAAGTAATAGGAAAAAATGGAAAGCTGATTCAGGAG
ATTGTGGACAAGTCAGGAGTTGTGAGGGTGAGGATTGAGGCTGAAAATGAGAAAAATGTTCCACAAGA
AGAGGAAATTATGCCACCAAATTCCCTTCCTTCCAATAATTCAAGGGTTGGACCTAATGCCCCAGAAG
AAAAAAAACATTTAGATATAAAGGAAAACAGCACCCATTTTTCTCAACCTAACAGTACAAAAGTCCAG
AGGGGTATGGTACCATTTGTTTTTGTGGGAACAAAGGACAGCATCGCTAATGCCACTGTTCTTTTGGA
TTATCACCTGAACTATTTAAAGGAAGTAGACCAGTTGCGTTTGGAGAGATTACAAATTGATGAGCAGT
TGCGACAGATTGGAGCTAGTTCTAGACCACCACCAAATCGTACAGATAAGGAAAAAAGCTATGTGACT
GATGATGGTCAAGGAATGGGTCGAGGTAGTAGACCTTACAGAAATAGGGGGCACGGCAGACGCGGTCC
TGGATATACTTCAGGAACTAATTCTGAAGCATCAAATGCTTCTGAAACAGAATCTGACCACAGAGACG
AACTCAGTGATTGGTCATTAGCTCCAACAGAGGAAGAGAGGGAGAGCTTCCTGCGCAGAGGAGACGGA
CGGCGGCGTGGAGGGGGAGGAAGAGGACAAGGAGGAAGAGGACGTGGAGGAGGCTTCAAAGGAAACGA
CGATCACTCCCGAACAGATAATCGTCCACGTAATCCAAGAGAGGCTAAAGGAAGAACAACAGATGGAT
CGCTTCAGATCAGAGTTGACTGCAATAATGAAAGGAGTGTCCACACTAAAACATTACAGAATACCTCC
AGTGAAGGTAGTCGGCTGCGCACGGGTAAAGATCGTAACCAGAAGAAAGAGAAGCCAGACAGCGTGGA
TGGTCAGCAACCACTCGTGAATGGAGTACCCGGATCCGGGCCCTTCGAACAAAAACTCATCTCAGAAG
AGGATCTGGTCGACTAGCGTACGGTCATTTCATGTCCTGTGTCAGTTTATGTTTTGGTCCACTTTTCC
AGTATTTTAGTGGACCCTGAAATGTGTGTGATGTGACATTTGTCATTTTCATTAGCAAAAAAAGTTGT
ATGATCTGTGCCTTTTTTATATCTTGGCAGGTAGGAATATTATATTTGGATGCAGAGTTCAGGGAAGA
TAAGTTGGAAACACTAAATGTTAAAGATGTAGCAAACCCTGTCAAACATTAGTACTTTATAGAAGAAT
GCATGCTTTCCATATTTTTTTCCTTACATAAACATCAGGTTAGGCAGTATAAAGAATAGGACTTGTTT
TTGTTTTTGTTTTGTTGCACTGAAGTTTGATAAATAGTGTTATTGAGAGAGATGTGTAATTTTTCTGT
ATAGACAGGAGAAGAAAGAACTATCTTCATCTGAGAGAGGCTAAAATGTTTTCAGCTAGGAACAAATC
TTCCTGGTCGAAAGTTAGTAGGATATGCCTGCTCTTTGGCCTGATGACCAATTTTAACTTAGAGCTTT
TTTTTTTTAATTTTGTCTGCCCCAAGTTTTGTGAAATTTTTCATATTTTAATTTCAACCTTATTTTGG
AGAGATAGGAAGGTCATTTCCATGTATGCATAATAATCCTGCAAAGTACAGGTACTTTGTCTAAGAAA
CATTGGAAGCAGGTTAAATGTTTTGTAAACTTTGAAATATATGGTCTAATGTTTAAGCAGAATTGGAA
AAGACTAAGATCGGTTAACAAATAACAACTTTTTTTTCTTTTTTTCTTTTGTTTTTTGAAGTGTTGGG
GTTTGGTTTTGTTTTTTGAGTCTTTTTTTTTTAAGTGAAATTTATTGAGGAAAAATATGTGAAGGACC
TTCACTCTAAGATGTTATATTTTTCTTAAAAAGTAACTCCTAGTAGGGGTACCACTGAATCTGTACAG
AGCCGTAAAAACTGAAGTTCTGCCTCTGATGTATTTTGTGAGTTTGTTTCTTTGAATTTTCATTTTAC
AGTTACTTTTCCTTGCATACAAACAAGCATATAAAATGGCAACAAACTGCACATGATTTCACAAATAT
TAAAAAGTCTTTTAAAAAGTATTGCCAAACATTAATGTTGATTTCTAGTTATTTATTCTGGGAATGTA
TAGTATTTGAAAACAGAAATTGGTACCTTGCACACATCATCTGTAAGCTGTTTGGTTTTAAAATACTG
TAGATAATTAACCAAGGTAGAATGACCTTGTAATGTAACTGCTCTTGGGCAATATTCTCTGTACATAT
TAGCGACAACAGATTGGATTTTATGTTGACATTTGTTTGGTTATAGTGCAATATATTTTGTATGCAAG
CAGTTTCAATAAAGTTTGATCTTCCTCTGCTAAATTGATGTTGATGCAATCCTTACAAATGATGCGGC
CGCAGGAACCCCTAGTGATGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAGCGAGCGAGCGCGCAGCTG
CCTGCAGGGGCGCCTGATGCGGTATTTTCTCCTTACGCATCTGTGCGGTATTTCACACCGCATACGTC
AAAGCAACCATAGTACGCGCCCTGTAGCGGCGCATTAAGCGCGGCGGGTGTGGTGGTTACGCGCAGCG
TGACCGCTACACTTGCCAGCGCCTTAGCGCCCGCTCCTTTCGCTTTCTTCCCTTCCTTTCTCGCCACG
TTCGCCGGCTTTCCCCGTCAAGCTCTAAATCGGGGGCTCCCTTTAGGGTTCCGATTTAGTGCTTTACG
GCACCTCGACCCCAAAAAACTTGATTTGGGTGATGGTTCACGTAGTGGGCCATCGCCCTGATAGACGG
TTTTTCGCCCTTTGACGTTGGAGTCCACGTTCTTTAATAGTGGACTCTTGTTCCAAACTGGAACAACA
CTCAACTCTATCTCGGGCTATTCTTTTGATTTATAAGGGATTTTGCCGATTTCGGTCTATTGGTTAAA
AAATGAGCTGATTTAACAAAAATTTAACGCGAATTTTAACAAAATATTAACGTTTACAATTTTATGGT
GCACTCTCAGTACAATCTGCTCTGATGCCGCATAGTTAAGCCAGCCCCGACACCCGCCAACACCCGCT
GACGCGCCCTGACGGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGGCCTCGTGATACGCC
TATTTTTATAGGTTAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCTCATGAGACAATA
ACCCTGATAAATGCTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGGTGAAAGTAAAA
GATGCTGAAGATCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTTGCGCAAACT
ATTAACTGGCGAACTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAAATCTGGAGCCGGT
GAGCGTGGAAGCCGCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGATTTAAAACTTCAT
TTTTAATTTAAAAGGATCTAGGTGAAGATCCTTTTTGATAATCTCATGACCAAAATCCCTTAACGTGA
GTTTTCGTTCCACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAATACTGTTCTTCT
AGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATTTTTGTGATG
CTCGTCAGGGGGGCGGAGCCTATGGAAAA
>hFMP1050-hFMR1-Myc-FMpA1 (SEQ ID NO: 2)
CTCTGGCACAGGGTGCACATACAGTAGGGGCAGAAATGAACCTCAAGTGCTTAACACAATTTTTAAAA
AATATATAGTCAAGTGAAAGTATGAAAATGAGTTGAGGAAAGGCGAGTACGTGGGTCAAAGCTGGGTC
TGAGGAAAGGCTCACATTTTGAGATCCCGACTCAATCCATGTCCCTTAAAGGGCACAGGGTGTCTCCA
CAGGGCCGCCCAAAATCTGGTGAGAGAGGGCGTAGACGCCTCACCTTCTGCCTCTACGGGTCACAAAA
GCCTGGGTCACCCTGGTTGCCACTGTTCCTAGTTCAAAGTCTTCTTCTGTCTAATCCTTCACCCCTAT
TCTCGCCTTCCACTCCACCTCCCGCTCAGTCAGACTGCGCTACTTTGAACCGGACCAAACCAAACCAA
ACCAAACCAAACCAAACCAGACCAGACACCCCCTCCCGCGGAATCCCAGAGAGGCCGAACTGGGATAA
CCGGATGCATTTGATTTCCCACGCCACTGAGTGCACCTCTGCAGAAATGGGCGTTCTGGCCCTCGCGA
GGCAGTGCGACCTGTCACCGCCCTTCAGCCTTCCCGCCCTCCACCAAGCCCGCGCACGCCCGGCCCGC
GCGTCTGTCTTTCGACCCGGCACCCCGGCCGGTTCCCAGCAGCGCGCATGCGCGCGCTCCCAGGCCAC
TTGAAGAGAGAGGGCGGGGCCGAGGGGCTGAGCCCGCGGGGGGAGGGAACAGCGTTGATCACGTGACG
TGGTTTCAGTGTTTACACCCGCAGCGGGCCGGGGGTTCGGCCTCAGTCAGGCGCTCAGCTCCGTTTCG
GTTTCACTTCCGGTGGAGGGCCGCCTCTGAGCGGGCGGCGGGCCGACGGCGAGCGCGGGCGGCGGCGG
TGACGGAGGCGCCGCTGCCAGGGGGCGTGCGGCAGCGCGGCGGCGGCGGCGGCGGCGGCGGCGGCGGA
GGCGGCGGCGGCGGCGGCGGCGGCGGCGGCTGGGCCTCGAGCGCCCGCAGCCCACCTCTCGGGGGCGG
GCTCCCGGCGCTAGCAGGGCTGAAGAGAAGATGGAGGAGCTGGTGGTGGAAGTGCGGGGCTCCAATGG
CGCTTTCTACAAGGCATTTGTAAAGGATGTTCATGAAGATTCAATAACAGTTGCATTTGAAAACAACT
GGCAGCCTGATAGGCAGATTCCATTTCATGATGTCAGATTCCCACCTCCTGTAGGTTATAATAAAGAT
ATAAATGAAAGTGATGAAGTTGAGGTGTATTCCAGAGCAAATGAAAAAGAGCCTTGCTGTTGGTGGTT
AGCTAAAGTGAGGATGATAAAGGGTGAGTTTTATGTGATAGAATATGCAGCATGTGATGCAACTTACA
ATGAAATTGTCACAATTGAACGTCTAAGATCTGTTAATCCCAACAAACCTGCCACAAAAGATACTTTC
CATAAGATCAAGCTGGATGTGCCAGAAGACTTACGGCAAATGTGTGCCAAAGAGGCGGCACATAAGGA
TTTTAAAAAGGCAGTTGGTGCCTTTTCTGTAACTTATGATCCAGAAAATTATCAGCTTGTCATTTTGT
CCATCAATGAAGTCACCTCAAAGCGAGCACATATGCTGATTGACATGCACTTTCGGAGTCTGCGCACT
AAGTTGTCTCTGATAATGAGAAATGAAGAAGCTAGTAAGCAGCTGGAGAGTTCAAGGCAGCTTGCCTC
GAGATTTCATGAACAGTTTATCGTAAGAGAAGATCTGATGGGTCTAGCTATTGGTACTCATGGTGCTA
ATATTCAGCAAGCTAGAAAAGTACCTGGGGTCACTGCTATTGATCTAGATGAAGATACCTGCACATTT
CATATTTATGGAGAGGATCAGGATGCAGTGAAAAAAGCTAGAAGTTTTCTCGAATTTGCTGAAGATGT
AATACAAGTTCCAAGGAACTTAGTAGGCAAAGTAATAGGAAAAAATGGAAAGCTGATTCAGGAGATTG
TGGACAAGTCAGGAGTTGTGAGGGTGAGGATTGAGGCTGAAAATGAGAAAAATGTTCCACAAGAAGAG
GAAATTATGCCACCAAATTCCCTTCCTTCCAATAATTCAAGGGTTGGACCTAATGCCCCAGAAGAAAA
AAAACATTTAGATATAAAGGAAAACAGCACCCATTTTTCTCAACCTAACAGTACAAAAGTCCAGAGGG
GTATGGTACCATTTGTTTTTGTGGGAACAAAGGACAGCATCGCTAATGCCACTGTTCTTTTGGATTAT
CACCTGAACTATTTAAAGGAAGTAGACCAGTTGCGTTTGGAGAGATTACAAATTGATGAGCAGTTGCG
ACAGATTGGAGCTAGTTCTAGACCACCACCAAATCGTACAGATAAGGAAAAAAGCTATGTGACTGATG
ATGGTCAAGGAATGGGTCGAGGTAGTAGACCTTACAGAAATAGGGGGCACGGCAGACGCGGTCCTGGA
TATACTTCAGGAACTAATTCTGAAGCATCAAATGCTTCTGAAACAGAATCTGACCACAGAGACGAACT
CAGTGATTGGTCATTAGCTCCAACAGAGGAAGAGAGGGAGAGCTTCCTGCGCAGAGGAGACGGACGGC
GGCGTGGAGGGGGAGGAAGAGGACAAGGAGGAAGAGGACGTGGAGGAGGCTTCAAAGGAAACGACGAT
CACTCCCGAACAGATAATCGTCCACGTAATCCAAGAGAGGCTAAAGGAAGAACAACAGATGGATCGCT
TCAGATCAGAGTTGACTGCAATAATGAAAGGAGTGTCCACACTAAAACATTACAGAATACCTCCAGTG
AAGGTAGTCGGCTGCGCACGGGTAAAGATCGTAACCAGAAGAAAGAGAAGCCAGACAGCGTGGATGGT
CAGCAACCACTCGTGAATGGAGTACCCGGATCCGGGCCCTTCGAACAAAAACTCATCTCAGAAGAGGA
TCTGGTCGACTAGCGTACGGTCATTTCATGTCCTGTGTCAGTTTATGTTTTGGTCCACTTTTCCAGTA
TTTTAGTGGACCCTGAAATGTGTGTGATGTGACATTTGTCATTTTCATTAGCAAAAAAAGTTGTATGA
TCTGTGCCTTTTTTATATCTTGGCAGGTAGGAATATTATATTTGGATGCAGAGTTCAGGGAAGATAAG
TTGGAAACACTAAATGTTAAAGATGTAGCAAACCCTGTCAAACATTAGTACTTTATAGAAGAATGCAT
GCTTTCCATATTTTTTTCCTTACATAAACATCAGGTTAGGCAGTATAAAGAATAGGACTTGTTTTTGT
TTTTGTTTTGTTGCACTGAAGTTTGATAAATAGTGTTATTGAGAGAGATGTGTAATTTTTCTGTATAG
ACAGGAGAAGAAAGAACTATCTTCATCTGAGAGAGGCTAAAATGTTTTCAGCTAGGAACAAATCTTCC
TGGTCGAAAGTTAGTAGGATATGCCTGCTCTTTGGCCTGATGACCAATTTTAACTTAGAGCTTTTTTT
TTTTAATTTTGTCTGCCCCAAGTTTTGTGAAATTTTTCATATTTTAATTTCAACCTTATTTTGGAGAG
ATAGGAAGGTCATTTCCATGTATGCATAATAATCCTGCAAAGTACAGGTACTTTGTCTAAGAAACATT
GGAAGCAGGTTAAATGTTTTGTAAACTTTGAAATATATGGTCTAATGTTTAAGCAGAATTGGAAAAGA
CTAAGATCGGTTAACAAATAACAACTTTTTTTTCTTTTTTTCTTTTGTTTTTTGAAGTGTTGGGGTTT
GGTTTTGTTTTTTGAGTCTTTTTTTTTTAAGTGAAATTTATTGAGGAAAAATATGTGAAGGACCTTCA
CTCTAAGATGTTATATTTTTCTTAAAAAGTAACTCCTAGTAGGGGTACCACTGAATCTGTACAGAGCC
GTAAAAACTGAAGTTCTGCCTCTGATGTATTTTGTGAGTTTGTTTCTTTGAATTTTCATTTTACAGTT
ACTTTTCCTTGCATACAAACAAGCATATAAAATGGCAACAAACTGCACATGATTTCACAAATATTAAA
AAGTCTTTTAAAAAGTATTGCCAAACATTAATGTTGATTTCTAGTTATTTATTCTGGGAATGTATAGT
ATTTGAAAACAGAAATTGGTACCTTGCACACATCATCTGTAAGCTGTTTGGTTTTAAAATACTGTAGA
TAATTAACCAAGGTAGAATGACCTTGTAATGTAACTGCTCTTGGGCAATATTCTCTGTACATATTAGC
GACAACAGATTGGATTTTATGTTGACATTTGTTTGGTTATAGTGCAATATATTTTGTATGCAAGCAGT
TTCAATAAAGTTTGATCTTCCTCTGCTAAATTGATGTTGATGCAATCCTTACAAATGAT
>hFMP1050 (human core promoter, SEQ ID NO: 3)
CTCTGGCACAGGGTGCACATACAGTAGGGGCAGAAATGAACCTCAAGTGCTTAACACAATTTTTAAAA
AATATATAGTCAAGTGAAAGTATGAAAATGAGTTGAGGAAAGGCGAGTACGTGGGTCAAAGCTGGGTC
TGAGGAAAGGCTCACATTTTGAGATCCCGACTCAATCCATGTCCCTTAAAGGGCACAGGGTGTCTCCA
CAGGGCCGCCCAAAATCTGGTGAGAGAGGGCGTAGACGCCTCACCTTCTGCCTCTACGGGTCACAAAA
GCCTGGGTCACCCTGGTTGCCACTGTTCCTAGTTCAAAGTCTTCTTCTGTCTAATCCTTCACCCCTAT
TCTCGCCTTCCACTCCACCTCCCGCTCAGTCAGACTGCGCTACTTTGAACCGGACCAAACCAAACCAA
ACCAAACCAAACCAAACCAGACCAGACACCCCCTCCCGCGGAATCCCAGAGAGGCCGAACTGGGATAA
CCGGATGCATTTGATTTCCCACGCCACTGAGTGCACCTCTGCAGAAATGGGCGTTCTGGCCCTCGCGA
GGCAGTGCGACCTGTCACCGCCCTTCAGCCTTCCCGCCCTCCACCAAGCCCGCGCACGCCCGGCCCGC
GCGTCTGTCTTTCGACCCGGCACCCCGGCCGGTTCCCAGCAGCGCGCATGCGCGCGCTCCCAGGCCAC
TTGAAGAGAGAGGGCGGGGCCGAGGGGCTGAGCCCGCGGGGGGAGGGAACAGCGTTGATCACGTGACG
TGGTTTCAGTGTTTACACCCGCAGCGGGCCGGGGGTTCGGCCTCAGTCAGGCGCTCAGCTCCGTTTCG
GTTTCACTTCCGGTGGAGGGCCGCCTCTGAGCGGGCGGCGGGCCGACGGCGAGCGCGGGCGGCGGCGG
TGACGGAGGCGCCGCTGCCAGGGGGCGTGCGGCAGCGCGGCGGCGGCGGCGGCGGCGGCGGCGGCGGA
GGCGGCGGCGGCGGCGGCGGCGGCGGCGGCTGGGCCTCGAGCGCCCGCAGCCCACCTCTCGGGGGCGG
GCTCCCGGCGCTAGCAGGGCTGAAGAGAAG
>hFMR1 (human isoform 7, SEQ ID NO: 4)
ATGGAGGAGCTGGTGGTGGAAGTGCGGGGCTCCAATGGCGCTTTCTACAAGGCATTTGTAAAGGATGT
TCATGAAGATTCAATAACAGTTGCATTTGAAAACAACTGGCAGCCTGATAGGCAGATTCCATTTCATG
ATGTCAGATTCCCACCTCCTGTAGGTTATAATAAAGATATAAATGAAAGTGATGAAGTTGAGGTGTAT
TCCAGAGCAAATGAAAAAGAGCCTTGCTGTTGGTGGTTAGCTAAAGTGAGGATGATAAAGGGTGAGTT
TTATGTGATAGAATATGCAGCATGTGATGCAACTTACAATGAAATTGTCACAATTGAACGTCTAAGAT
CTGTTAATCCCAACAAACCTGCCACAAAAGATACTTTCCATAAGATCAAGCTGGATGTGCCAGAAGAC
TTACGGCAAATGTGTGCCAAAGAGGCGGCACATAAGGATTTTAAAAAGGCAGTTGGTGCCTTTTCTGT
AACTTATGATCCAGAAAATTATCAGCTTGTCATTTTGTCCATCAATGAAGTCACCTCAAAGCGAGCAC
ATATGCTGATTGACATGCACTTTCGGAGTCTGCGCACTAAGTTGTCTCTGATAATGAGAAATGAAGAA
GCTAGTAAGCAGCTGGAGAGTTCAAGGCAGCTTGCCTCGAGATTTCATGAACAGTTTATCGTAAGAGA
AGATCTGATGGGTCTAGCTATTGGTACTCATGGTGCTAATATTCAGCAAGCTAGAAAAGTACCTGGGG
TCACTGCTATTGATCTAGATGAAGATACCTGCACATTTCATATTTATGGAGAGGATCAGGATGCAGTG
AAAAAAGCTAGAAGTTTTCTCGAATTTGCTGAAGATGTAATACAAGTTCCAAGGAACTTAGTAGGCAA
AGTAATAGGAAAAAATGGAAAGCTGATTCAGGAGATTGTGGACAAGTCAGGAGTTGTGAGGGTGAGGA
TTGAGGCTGAAAATGAGAAAAATGTTCCACAAGAAGAGGAAATTATGCCACCAAATTCCCTTCCTTCC
AATAATTCAAGGGTTGGACCTAATGCCCCAGAAGAAAAAAAACATTTAGATATAAAGGAAAACAGCAC
CCATTTTTCTCAACCTAACAGTACAAAAGTCCAGAGGGGTATGGTACCATTTGTTTTTGTGGGAACAA
AGGACAGCATCGCTAATGCCACTGTTCTTTTGGATTATCACCTGAACTATTTAAAGGAAGTAGACCAG
TTGCGTTTGGAGAGATTACAAATTGATGAGCAGTTGCGACAGATTGGAGCTAGTTCTAGACCACCACC
AAATCGTACAGATAAGGAAAAAAGCTATGTGACTGATGATGGTCAAGGAATGGGTCGAGGTAGTAGAC
CTTACAGAAATAGGGGGCACGGCAGACGCGGTCCTGGATATACTTCAGGAACTAATTCTGAAGCATCA
AATGCTTCTGAAACAGAATCTGACCACAGAGACGAACTCAGTGATTGGTCATTAGCTCCAACAGAGGA
AGAGAGGGAGAGCTTCCTGCGCAGAGGAGACGGACGGCGGCGTGGAGGGGGAGGAAGAGGACAAGGAG
GAAGAGGACGTGGAGGAGGCTTCAAAGGAAACGACGATCACTCCCGAACAGATAATCGTCCACGTAAT
CCAAGAGAGGCTAAAGGAAGAACAACAGATGGATCGCTTCAGATCAGAGTTGACTGCAATAATGAAAG
GAGTGTCCACACTAAAACATTACAGAATACCTCCAGTGAAGGTAGTCGGCTGCGCACGGGTAAAGATC
GTAACCAGAAGAAAGAGAAGCCAGACAGCGTGGATGGTCAGCAACCACTCGTGAATGGAGTACCCTAG
>FMpA1 (endogenous human 3′UTR and polyA, SEQ ID NO : 5)
GTCATTTCATGTCCTGTGTCAGTTTATGTTTTGGTCCACTTTTCCAGTATTTTAGTGGACCCTGAAAT
GTGTGTGATGTGACATTTGTCATTTTCATTAGCAAAAAAAGTTGTATGATCTGTGCCTTTTTTATATC
TTGGCAGGTAGGAATATTATATTTGGATGCAGAGTTCAGGGAAGATAAGTTGGAAACACTAAATGTTA
AAGATGTAGCAAACCCTGTCAAACATTAGTACTTTATAGAAGAATGCATGCTTTCCATATTTTTTTCC
TTACATAAACATCAGGTTAGGCAGTATAAAGAATAGGACTTGTTTTTGTTTTTGTTTTGTTGCACTGA
AGTTTGATAAATAGTGTTATTGAGAGAGATGTGTAATTTTTCTGTATAGACAGGAGAAGAAAGAACTA
TCTTCATCTGAGAGAGGCTAAAATGTTTTCAGCTAGGAACAAATCTTCCTGGTCGAAAGTTAGTAGGA
TATGCCTGCTCTTTGGCCTGATGACCAATTTTAACTTAGAGCTTTTTTTTTTTAATTTTGTCTGCCCC
AAGTTTTGTGAAATTTTTCATATTTTAATTTCAACCTTATTTTGGAGAGATAGGAAGGTCATTTCCAT
GTATGCATAATAATCCTGCAAAGTACAGGTACTTTGTCTAAGAAACATTGGAAGCAGGTTAAATGTTT
TGTAAACTTTGAAATATATGGTCTAATGTTTAAGCAGAATTGGAAAAGACTAAGATCGGTTAACAAAT
AACAACTTTTTTTTCTTTTTTTCTTTTGTTTTTTGAAGTGTTGGGGTTTGGTTTTGTTTTTTGAGTCT
TTTTTTTTTAAGTGAAATTTATTGAGGAAAAATATGTGAAGGACCTTCACTCTAAGATGTTATATTTT
TCTTAAAAAGTAACTCCTAGTAGGGGTACCACTGAATCTGTACAGAGCCGTAAAAACTGAAGTTCTGC
CTCTGATGTATTTTGTGAGTTTGTTTCTTTGAATTTTCATTTTACAGTTACTTTTCCTTGCATACAAA
CAAGCATATAAAATGGCAACAAACTGCACATGATTTCACAAATATTAAAAAGTCTTTTAAAAAGTATT
GCCAAACATTAATGTTGATTTCTAGTTATTTATTCTGGGAATGTATAGTATTTGAAAACAGAAATTGG
TACCTTGCACACATCATCTGTAAGCTGTTTGGTTTTAAAATACTGTAGATAATTAACCAAGGTAGAAT
GACCTTGTAATGTAACTGCTCTTGGGCAATATTCTCTGTACATATTAGCGACAACAGATTGGATTTTA
TGTTGACATTTGTTTGGTTATAGTGCAATATATTTTGTATGCAAGCAGTTTCAATAAAGTTTGATCTT
CCTCTGCTAAATTGATGTTGATGCAATCCTTACAAATGAT
SEQ ID NO: 6 Human FMRP amino acid sequence
MEELVVEVRGSNGAFYKAFVKDVHEDSITVAFENNWQPDRQIPFHDVRFPPPVGYNKDINESDEVEVY
SRANEKEPCCWWLAKVRMIKGEFYVIEYAACDATYNEIVTIERLRSVNPNKPATKDTFHKIKLDVPED
LRQMCAKEAAHKDFKKAVGAFSVTYDPENYQLVILSINEVTSKRAHMLIDMHFRSLRTKLSLIMRNEE
ASKQLESSRQLASRFHEQFIVREDLMGLAIGTHGANIQQARKVPGVTAIDLDEDTCTFHIYGEDQDAV
KKARSFLEFAEDVIQVPRNLVGKVIGKNGKLIQEIVDKSGVVRVRIEAENEKNVPQEEEIMPPNSLPS
NNSRVGPNAPEEKKHLDIKENSTHFSQPNSTKVQRGMVPFVFVGTKDSIANATVLLDYHLNYLKEVDQ
LRLERLQIDEQLRQIGASSRPPPNRTDKEKSYVTDDGQGMGRGSRPYRNRGHGRRGPGYTSGTNSEAS
NASETESDHRDELSDWSLAPTEEERESFLRRGDGRRRGGGGRGQGGRGRGGGFKGNDDHSRTDNRPRN
PREAKGRTTDGSLQIRVDCNNERSVHTKTLQNTSSEGSRLRTGKDRNQKKEKPDSVDGQQPLVNGVP
>CBh-hFMR1-bGHpA (SEQ ID NO: 7)
CGTTACATAACTTACGGTAAATGGCCCGCCTGGCTGACCGCCCAACGACCCCCGCCCATTGACGTCAA
TAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGC
CTGGCATTGTGCCCAGTACATGACCTTATGGGACTTTCCTACTTGGCAGTACATCTACGTATTAGTCA
TCGCTATTACCATGGTCGAGGTGAGCCCCACGTTCTGCTTCACTCTCCCCATCTCCCCCCCCTCCCCA
CCCCCAATTTTGTATTTATTTATTTTTTAATTATTTTGTGCAGCGATGGGGGCGGGGGGGGGGGGGGG
GCGCGCGCCAGGCGGGGCGGGGCGGGGCGAGGGGCGGGGCGGGGCGAGGCGGAGAGGTGCGGCGGCAG
CCAATCAGAGCGGCGCGCTCCGAAAGTTTCCTTTTATGGCGAGGCGGCGGCGGCGGCGGCCCTATAAA
AAGCGAAGCGCGCGGCGGGCGGGAGTCGCTGCGCGCTGCCTTCGCCCCGTGCCCCGCTCCGCCGCCGC
CTCGCGCCGCCCGCCCCGGCTCTGACTGACCGCGTTACTCCCACAGGTGAGCGGGCGGGACGGCCCTT
CTCCTCCGGGCTGTAATTAGCTGAGCAAGAGGTAAGGGTTTAAGGGATGGTTGGTTGGTGGGGTATTA
ATGTTTAATTACCTGGAGCACCTGCCTGAAATCACTTTTTTTCAGGTTGGACCGGTGCCACCATGGAG
GAGCTGGTGGTGGAAGTGCGGGGCTCCAATGGCGCTTTCTACAAGGCATTTGTAAAGGATGTTCATGA
AGATTCAATAACAGTTGCATTTGAAAACAACTGGCAGCCTGATAGGCAGATTCCATTTCATGATGTCA
GATTCCCACCTCCTGTAGGTTATAATAAAGATATAAATGAAAGTGATGAAGTTGAGGTGTATTCCAGA
GCAAATGAAAAAGAGCCTTGCTGTTGGTGGTTAGCTAAAGTGAGGATGATAAAGGGTGAGTTTTATGT
GATAGAATATGCAGCATGTGATGCAACTTACAATGAAATTGTCACAATTGAACGTCTAAGATCTGTTA
ATCCCAACAAACCTGCCACAAAAGATACTTTCCATAAGATCAAGCTGGATGTGCCAGAAGACTTACGG
CAAATGTGTGCCAAAGAGGCGGCACATAAGGATTTTAAAAAGGCAGTTGGTGCCTTTTCTGTAACTTA
TGATCCAGAAAATTATCAGCTTGTCATTTTGTCCATCAATGAAGTCACCTCAAAGCGAGCACATATGC
TGATTGACATGCACTTTCGGAGTCTGCGCACTAAGTTGTCTCTGATAATGAGAAATGAAGAAGCTAGT
AAGCAGCTGGAGAGTTCAAGGCAGCTTGCCTCGAGATTTCATGAACAGTTTATCGTAAGAGAAGATCT
GATGGGTCTAGCTATTGGTACTCATGGTGCTAATATTCAGCAAGCTAGAAAAGTACCTGGGGTCACTG
CTATTGATCTAGATGAAGATACCTGCACATTTCATATTTATGGAGAGGATCAGGATGCAGTGAAAAAA
GCTAGAAGTTTTCTCGAATTTGCTGAAGATGTAATACAAGTTCCAAGGAACTTAGTAGGCAAAGTAAT
AGGAAAAAATGGAAAGCTGATTCAGGAGATTGTGGACAAGTCAGGAGTTGTGAGGGTGAGGATTGAGG
CTGAAAATGAGAAAAATGTTCCACAAGAAGAGGAAATTATGCCACCAAATTCCCTTCCTTCCAATAAT
TCAAGGGTTGGACCTAATGCCCCAGAAGAAAAAAAACATTTAGATATAAAGGAAAACAGCACCCATTT
TTCTCAACCTAACAGTACAAAAGTCCAGAGGGGTATGGTACCATTTGTTTTTGTGGGAACAAAGGACA
GCATCGCTAATGCCACTGTTCTTTTGGATTATCACCTGAACTATTTAAAGGAAGTAGACCAGTTGCGT
TTGGAGAGATTACAAATTGATGAGCAGTTGCGACAGATTGGAGCTAGTTCTAGACCACCACCAAATCG
TACAGATAAGGAAAAAAGCTATGTGACTGATGATGGTCAAGGAATGGGTCGAGGTAGTAGACCTTACA
GAAATAGGGGGCACGGCAGACGCGGTCCTGGATATACTTCAGGAACTAATTCTGAAGCATCAAATGCT
TCTGAAACAGAATCTGACCACAGAGACGAACTCAGTGATTGGTCATTAGCTCCAACAGAGGAAGAGAG
GGAGAGCTTCCTGCGCAGAGGAGACGGACGGCGGCGTGGAGGGGGAGGAAGAGGACAAGGAGGAAGAG
GACGTGGAGGAGGCTTCAAAGGAAACGACGATCACTCCCGAACAGATAATCGTCCACGTAATCCAAGA
GAGGCTAAAGGAAGAACAACAGATGGATCGCTTCAGATCAGAGTTGACTGCAATAATGAAAGGAGTGT
CCACACTAAAACATTACAGAATACCTCCAGTGAAGGTAGTCGGCTGCGCACGGGTAAAGATCGTAACC
AGAAGAAAGAGAAGCCAGACAGCGTGGATGGTCAGCAACCACTCGTGAATGGAGTACCCGGATCCGGG
CCCTTCGAACAAAAACTCATCTCAGAAGAGGATCTGGTCGACTAGCGTACGCTGTGCCTTCTAGTTGC
CAGCCATCTGTTGTTTGCCCCTCCCCCGTGCCTTCCTTGACCCTGGAAGGTGCCACTCCCACTGTCCT
TTCCTAATAAAATGAGGAAATTGCATCGCATTGTCTGAGTAGGTGTCATTCTATTCTGGGGGGTGGGG
TGGGGCAGGACAGCAAGGGGGAGGATTGGGAAGACAATAGCAGGCATGCTGGGGAACTAGTAGGGTAG
CTAGCATGGAAAAACAAAGCAATTTACATAGAGTTTTAATGGATCTCCAGAATCAGAAACTGAAAGAG
TTGAATGACTGGCTAACAAAAACAGAAGAAAGAACAAGGAAAATGGAGGAAGAGCCTCTTGGACCTGA
TCTTGAAGACCTAAAACGCCAAGTACAACAACATAAGGTGCTTCAAGAAGATCTAGAACAAGAACAAG
TCAGGGTCAATTCTCTCACTCACATGGTGGTGGTAGTTGATGAATCTAGTGGAGATCACGCAACTGCT
GCTTTGGAAGAACAACTTAAGGTATTGGGAGATCGATGGGCAAACATCTGTAGATGGACAGAAGACCG
CTGGGTTCTTTTACAAGACCAGCCTGACCTAGCTCCTGGACTGACCACTATTGGAGCCTCTCCTACTC
AGACTGTTACTCTGGTGACACAACCTGTGGTTACTAAGGAAACTGCCATCTCCAAACTAGAAATGCCA
TCTTCCTTGATGTTGGAGGTACCTACTCATAGATTACTGCAACAGTTCCCCCTGGACCTGGAAAAGTT
TCTTGCCTGGCTTACAGAAGCTGAAACAACTGCCAATGTCCTACAGGATGCTACCCGTAAGGAAAGGC
TCCTAGAAGACTCCAAGGGAGTAAAAGAGCTGATGAAACAATGGCAAGACCTCCAAGGTGAAATTGAA
GCTCACACAGATGTTTATCACAACCTGGATGAAAACAGCCAAAAAATCCTGAGATCCCTGGAAGGTTC
CGATGATGCAGTCCTGTTACAAAGACGTTTGGATAACATGAACTTCAAGTGGAGTGAACTTCGGAAAA
AGTCTCTCAACATTAGGTCCCATTTGGAAGCCAGTTCTGACCAGTGGAAGCGTCTGCACCTTTCTCTG
CAGGAACTTCTGGTGTGGCTACAGCTGAAAGATGATGAATTAAGCCGGCAGGCACCTATTGGAGGCGA
CTTTCCAGCAGTTCAGAAGCAGAACGATGTACATAGGGCCTTCAAGAGGGAATTGAAAACTAAAGAAC
CTGTAATCATGAGTACTCTTGAGACTGTACGAATATTTCTGACAGAGCAGCCTTTGGAAGGACTAGAG
AAACTCTACCAGGAGCCCAGAGAGCTGCCTCCTGAGGAGAGAGCCCAGAATGTCACTCGGCTTCTACG
AAAGCAGGCTGAGGAGGTCAATACTGAGTGGGAAAAA
>hFMP1050-hFMR1-FMpA1 plasmid (SEQ ID NO: 8)
•hFMP1050 promoter sequence in bold
•hFMR1 GOI sequence in bold and underlined
•FMpA1 sequence underlined
TGTGGGCGGACAAAATAGTTGGGAACTGGGAGGGGTGGAAATGGAGTTTTTAAGGATTATTT
AGGGAAGAGTGACAAAATAGATGGGAACTGGGTGTAGCGTCGTAAGCTAATACGAAAATTAA
AAATGACAAAATAGTTTGGAACTAGATTTCACTTATCTGGTTCGGATCTCCTAGAGCTTACA
TTATTGAAGCATTTATCAGGGTTATTGTCTCAGACCTGCAGGCAGCTGCGCGCTCGCTCGCT
CACTGAGGCCGCCCGGGCAAAGCCCGGGCGTCGGGCGACCTTTGGTCGCCCGGCCTCAGTGA
GCGAGCGAGCGCGCAGAGAGGGAGTGGCCAACTCCATCACTAGGGGTTCCTGCGGCCGCACG
CGTACTAGTCTCTGGCACAGGGTGCACATACAGTAGGGGCAGAAATGAACCTCAAGTGCTTA
ACACAATTTTTAAAAAATATATAGTCAAGTGAAAGTATGAAAATGAGTTGAGGAAAGGCGAG
TACGTGGGTCAAAGCTGGGTCTGAGGAAAGGCTCACATTTTGAGATCCCGACTCAATCCATG
TCCCTTAAAGGGCACAGGGTGTCTCCACAGGGCCGCCCAAAATCTGGTGAGAGAGGGCGTAG
ACGCCTCACCTTCTGCCTCTACGGGTCACAAAAGCCTGGGTCACCCTGGTTGCCACTGTTCC
TAGTTCAAAGTCTTCTTCTGTCTAATCCTTCACCCCTATTCTCGCCTTCCACTCCACCTCCC
GCTCAGTCAGACTGCGCTACTTTGAACCGGACCAAACCAAACCAAACCAAACCAAACCAAAC
CAGACCAGACACCCCCTCCCGCGGAATCCCAGAGAGGCCGAACTGGGATAACCGGATGCATT
TGATTTCCCACGCCACTGAGTGCACCTCTGCAGAAATGGGCGTTCTGGCCCTCGCGAGGCAG
TGCGACCTGTCACCGCCCTTCAGCCTTCCCGCCCTCCACCAAGCCCGCGCACGCCCGGCCCG
CGCGTCTGTCTTTCGACCCGGCACCCCGGCCGGTTCCCAGCAGCGCGCATGCGCGCGCTCCC
AGGCCACTTGAAGAGAGAGGGCGGGGCCGAGGGGCTGAGCCCGCGGGGGGAGGGAACAGCGT
TGATCACGTGACGTGGTTTCAGTGTTTACACCCGCAGCGGGCCGGGGGTTCGGCCTCAGTCA
GGCGCTCAGCTCCGTTTCGGTTTCACTTCCGGTGGAGGGCCGCCTCTGAGCGGGCGGCGGGC
CGACGGCGAGCGCGGGCGGCGGCGGTGACGGAGGCGCCGCTGCCAGGGGGCGTGCGGCAGCG
CGGCGGCGGCGGCGGCGGCGGCGGCGGCGGAGGCGGCGGCGGCGGCGGCGGCGGCGGCGGCT
GGGCCTCGAGCGCCCGCAGCCCACCTCTCGGGGGCGGGCTCCCGGCGCTAGCAGGGCTGAAG
AGAAGATGGAGGAGCTGGTGGTGGAAGTGCGGGGCTCCAATGGCGCTTTCTACAAGGCATTT
GTAAAGGATGTTCATGAAGATTCAATAACAGTTGCATTTGAAAACAACTGGCAGCCTGATAG
GCAGATTCCATTTCATGATGTCAGATTCCCACCTCCTGTAGGTTATAATAAAGATATAAATG
AAAGTGATGAAGTTGAGGTGTATTCCAGAGCAAATGAAAAAGAGCCTTGCTGTTGGTGGTTA
GCTAAAGTGAGGATGATAAAGGGTGAGTTTTATGTGATAGAATATGCAGCATGTGATGCAAC
TTACAATGAAATTGTCACAATTGAACGTCTAAGATCTGTTAATCCCAACAAACCTGCCACAA
AAGATACTTTCCATAAGATCAAGCTGGATGTGCCAGAAGACTTACGGCAAATGTGTGCCAAA
GAGGCGGCACATAAGGATTTTAAAAAGGCAGTTGGTGCCTTTTCTGTAACTTATGATCCAGA
AAATTATCAGCTTGTCATTTTGTCCATCAATGAAGTCACCTCAAAGCGAGCACATATGCTGA
TTGACATGCACTTTCGGAGTCTGCGCACTAAGTTGTCTCTGATAATGAGAAATGAAGAAGCT
AGTAAGCAGCTGGAGAGTTCAAGGCAGCTTGCCTCGAGATTTCATGAACAGTTTATCGTAAG
AGAAGATCTGATGGGTCTAGCTATTGGTACTCATGGTGCTAATATTCAGCAAGCTAGAAAAG
TACCTGGGGTCACTGCTATTGATCTAGATGAAGATACCTGCACATTTCATATTTATGGAGAG
GATCAGGATGCAGTGAAAAAAGCTAGAAGTTTTCTCGAATTTGCTGAAGATGTAATACAAGT
TCCAAGGAACTTAGTAGGCAAAGTAATAGGAAAAAATGGAAAGCTGATTCAGGAGATTGTGG
ACAAGTCAGGAGTTGTGAGGGTGAGGATTGAGGCTGAAAATGAGAAAAATGTTCCACAAGAA
GAGGAAATTATGCCACCAAATTCCCTTCCTTCCAATAATTCAAGGGTTGGACCTAATGCCCC
AGAAGAAAAAAAACATTTAGATATAAAGGAAAACAGCACCCATTTTTCTCAACCTAACAGTA
CAAAAGTCCAGAGGGGTATGGTACCATTTGTTTTTGTGGGAACAAAGGACAGCATCGCTAAT
GCCACTGTTCTTTTGGATTATCACCTGAACTATTTAAAGGAAGTAGACCAGTTGCGTTTGGA
GAGATTACAAATTGATGAGCAGTTGCGACAGATTGGAGCTAGTTCTAGACCACCACCAAATC
GTACAGATAAGGAAAAAAGCTATGTGACTGATGATGGTCAAGGAATGGGTCGAGGTAGTAGA
CCTTACAGAAATAGGGGGCACGGCAGACGCGGTCCTGGATATACTTCAGGAACTAATTCTGA
AGCATCAAATGCTTCTGAAACAGAATCTGACCACAGAGACGAACTCAGTGATTGGTCATTAG
CTCCAACAGAGGAAGAGAGGGAGAGCTTCCTGCGCAGAGGAGACGGACGGCGGCGTGGAGGG
GGAGGAAGAGGACAAGGAGGAAGAGGACGTGGAGGAGGCTTCAAAGGAAACGACGATCACTC
CCGAACAGATAATCGTCCACGTAATCCAAGAGAGGCTAAAGGAAGAACAACAGATGGATCGC
TTCAGATCAGAGTTGACTGCAATAATGAAAGGAGTGTCCACACTAAAACATTACAGAATACC
TCCAGTGAAGGTAGTCGGCTGCGCACGGGTAAAGATCGTAACCAGAAGAAAGAGAAGCCAGA
CAGCGTGGATGGTCAGCAACCACTCGTGAATGGAGTACCCTGAGGATCCCCCAGTACATGAC
CTTATGGCCTAGGGTCATTTCATGTCCTGTGTCAGTTTATGTTTTGGTCCACTTTTCCAGTA
TTTTAGTGGACCCTGAAATGTGTGTGATGTGACATTTGTCATTTTCATTAGCAAAAAAAGTT
GTATGATCTGTGCCTTTTTTATATCTTGGCAGGTAGGAATATTATATTTGGATGCAGAGTTC
AGGGAAGATAAGTTGGAAACACTAAATGTTAAAGATGTAGCAAACCCTGTCAAACATTAGTA
CTTTATAGAAGAATGCATGCTTTCCATATTTTTTTCCTTACATAAACATCAGGTTAGGCAGT
ATAAAGAATAGGACTTGTTTTTGTTTTTGTTTTGTTGCACTGAAGTTTGATAAATAGTGTTA
TTGAGAGAGATGTGTAATTTTTCTGTATAGACAGGAGAAGAAAGAACTATCTTCATCTGAGA
GAGGCTAAAATGTTTTCAGCTAGGAACAAATCTTCCTGGTCGAAAGTTAGTAGGATATGCCT
GCTCTTTGGCCTGATGACCAATTTTAACTTAGAGCTTTTTTTTTTTAATTTTGTCTGCCCCA
AGTTTTGTGAAATTTTTCATATTTTAATTTCAACCTTATTTTGGAGAGATAGGAAGGTCATT
TCCATGTATGCATAATAATCCTGCAAAGTACAGGTACTTTGTCTAAGAAACATTGGAAGCAG
GTTAAATGTTTTGTAAACTTTGAAATATATGGTCTAATGTTTAAGCAGAATTGGAAAAGACT
AAGATCGGTTAACAAATAACAACTTTTTTTTCTTTTTTTCTTTTGTTTTTTGAAGTGTTGGG
GTTTGGTTTTGTTTTTTGAGTCTTTTTTTTTTAAGTGAAATTTATTGAGGAAAAATATGTGA
AGGACCTTCACTCTAAGATGTTATATTTTTCTTAAAAAGTAACTCCTAGTAGGGGTACCACT
GAATCTGTACAGAGCCGTAAAAACTGAAGTTCTGCCTCTGATGTATTTTGTGAGTTTGTTTC
TTTGAATTTTCATTTTACAGTTACTTTTCCTTGCATACAAACAAGCATATAAAATGGCAACA
AACTGCACATGATTTCACAAATATTAAAAAGTCTTTTAAAAAGTATTGCCAAACATTAATGT
TGATTTCTAGTTATTTATTCTGGGAATGTATAGTATTTGAAAACAGAAATTGGTACCTTGCA
CACATCATCTGTAAGCTGTTTGGTTTTAAAATACTGTAGATAATTAACCAAGGTAGAATGAC
CTTGTAATGTAACTGCTCTTGGGCAATATTCTCTGTACATATTAGCGACAACAGATTGGATT
TTATGTTGACATTTGTTTGGTTATAGTGCAATATATTTTGTATGCAAGCAGTTTCAATAAAG
TTTGATCTTCCTCTGCTAAATTGATGTTGATGCAATCCTTACAAATGATTCCGGACACGTGC
GGACCGAGCGGCCGCAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCCGGGCGGCCTC
AGTGAGCGAGCGAGCGCGCAGCTGCCTGCAGGCATGCAAGCTGTAGCCAACCACTAGAACTA
TAGCTAGAGTCCTGGGCGAACAAACGATGCTCGCCTTCCAGAAAACCGAGGATGCGAACCAC
TTCATCCGGGGTCAGCACCACCGGCAAGCGCCGCGACGGCCGAGGTCTTCCGATCTCCTGAA
GCCAGGGCAGATCCGTGCACAGCACCTTGCCGTAGAAGAACAGCAAGGCCGCCAATGCCTGA
CGATGCGTGGAGACCGAAACCTTGCGCTCGTTCGCCAGCCAGGACAGAAATGCCTCGACTTC
GCTGCTGCCCAAGGTTGCCGGGTGACGCACACCGTGGAAACGGATGAAGGCACGAACCCAGT
TGACATAAGCCTGTTCGGTTCGTAAACTGTAATGCAAGTAGCGTATGCGCTCACGCAACTGG
TCCAGAACCTTGACCGAACGCAGCGGTGGTAACGGCGCAGTGGCGGTTTTCATGGCTTGTTA
TGACTGTTTTTTTGTACAGTCTATGCCTCGGGCATCCAAGCAGCAAGCGCGTTACGCCGTGG
GTCGATGTTTGATGTTATGGAGCAGCAACGATGTTACGCAGCAGGGCAGTCGCCCTAAAACA
AAGTTAGGTGGCTCAAGTATGGGCATCATTCGCACATGTAGGCTCGGCCCTGACCAAGTCAA
ATCCATGCGGGCTGCTCTTGATCTTTTCGGTCGTGAGTTCGGAGACGTAGCCACCTACTCCC
AACATCAGCCGGACTCCGATTACCTCGGGAACTTGCTCCGTAGTAAGACATTCATCGCGCTT
GCTGCCTTCGACCAAGAAGCGGTTGTTGGCGCTCTCGCGGCTTACGTTCTGCCCAAGTTTGA
GCAGCCGCGTAGTGAGATCTATATCTATGATCTCGCAGTCTCCGGCGAGCACCGGAGGCAGG
GCATTGCCACCGCGCTCATCAATCTCCTCAAGCATGAGGCCAACGCGCTTGGTGCTTATGTG
ATCTACGTGCAAGCAGATTACGGTGACGATCCCGCAGTGGCTCTCTATACAAAGTTGGGCAT
ACGGGAAGAAGTGATGCACTTTGATATCGACCCAAGTACCGCCACCTAACAATTCGTTCAAG
CCGAGATCGGCTTCCCGGCCGCGGAGTTGTTCGGTAAATTGTCACAACGCCGCGAATATAGT
CTTTACCATGCCCTTGGCCACGCCCCTCTTTAATACGACGGGCAATTTGCACTTCAGAAAAT
GAAGAGTTTGCTTTAGCCATAACAAAAGTCCAGTATGCTTTTTCACAGCATAACTGGACTGA
TTTCAGTTTACAACTATTCTGTCTAGTTTAAGACTTTATTGTCATAGTTTAGATCTATTTTG
TTCAGTTTAAGACTTTATTGTCCGCCCACACCCGCTTACGCAGGGCATCCATTTATTACTCA
ACCGTAACCGATTTTGCCAGGTTACGCGGCTGGTCTATGCGGTGTGAAATACCGCACAGATG
CGTAAGGAGAAAATACCGCATCAGGCGCTCTTCCGCTTCCTCGCTCACTGACTCGCTGCGCT
CGGTCGTTCGGCTGCGGCGAGCGGTATCAGCTCACTCAAAGGCGGTAATACGGTTATCCACA
GAATCAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGA
AGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAGGGATTTTG
GTCATGAGATTATCAAAAAGGATCTTCACCTAGATCCTTTTAAATTAAAAATGAAGTTTTAA
ATCAATCTAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTCGTGTAG
ATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAA
GTGGTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGTCAT
GCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAAGGATCTT
ACCGCTGTTGAGATCCAGTTCGATGTAACCCACTCGTGCACCCAACTGATCTTCAGCATCTT
TTACTTTCACCAGCGTTTCTGGGTGAGCAAAAACAGGAAGGCAAAATGCCGCAAAAAAGGGA
ATAAGGGCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAATAAACAAA
TAGGGGTTCCGCGCACATTTCCCCGAAAAGTGCCACCTAAATTGTAAGCGTTAATATTTTGT
TAAAATTCGCGTTAAATTTTTGTTAAATCAGCTCATTTTTTAACCAATAGGCCGAAATCGGC
AAAATCCCTTATAAATCAAAAGAATAGACCGAGATAGGGTTGAGTGTTGTTCCAGTTTGGAA
CAAGAGTCCACTATTAAGAACGTGGACTCCAACGTCAAAGGGCGAAAAACCGTCTATCAGGG
CGATGGCCCACTACGTGAACCATCACCCTAATCAAGTTTTTTGGGGTCGAGGTGCCGTAAAG
CACTAAATCGGAACCCTAAAGGGAGCCCCCGATTTAGAGCTTGACGGGGAAAGCCGGCGAAC
GTGGCGAGAAAGGAAGGGAAGAAAGCGAAAGGAGCGGGCGCTAGGGCGCTGGCAAGTGTAGC
GGTCACGCTGCGCGTAACCACCACACCCGCCGCGCTTAATGCGCCGCTACAGGGCGCGTCCC
ATTCGCCATTCAGGCTGCAAATAAGCGTTGATATTCAGTCAATTACAAACATTAATAACGAA
GAGATGACAGAAAAATTTTCATTCTGTGACAGAGAAAAAGTAGCCGAAGATGACGGTTTGTC
ACATGGAGTTGGCAGGATGTTTGATTAAAAACATAACAGGAAGAAAAATGCCCCGC
>hFMP1050-hFMR1-FMpA1 (SEQ ID NO: 9)
CTCTGGCACAGGGTGCACATACAGTAGGGGCAGAAATGAACCTCAAGTGCTTAACACAATTT
TTAAAAAATATATAGTCAAGTGAAAGTATGAAAATGAGTTGAGGAAAGGCGAGTACGTGGGT
CAAAGCTGGGTCTGAGGAAAGGCTCACATTTTGAGATCCCGACTCAATCCATGTCCCTTAAA
GGGCACAGGGTGTCTCCACAGGGCCGCCCAAAATCTGGTGAGAGAGGGCGTAGACGCCTCAC
CTTCTGCCTCTACGGGTCACAAAAGCCTGGGTCACCCTGGTTGCCACTGTTCCTAGTTCAAA
GTCTTCTTCTGTCTAATCCTTCACCCCTATTCTCGCCTTCCACTCCACCTCCCGCTCAGTCA
GACTGCGCTACTTTGAACCGGACCAAACCAAACCAAACCAAACCAAACCAAACCAGACCAGA
CACCCCCTCCCGCGGAATCCCAGAGAGGCCGAACTGGGATAACCGGATGCATTTGATTTCCC
ACGCCACTGAGTGCACCTCTGCAGAAATGGGCGTTCTGGCCCTCGCGAGGCAGTGCGACCTG
TCACCGCCCTTCAGCCTTCCCGCCCTCCACCAAGCCCGCGCACGCCCGGCCCGCGCGTCTGT
CTTTCGACCCGGCACCCCGGCCGGTTCCCAGCAGCGCGCATGCGCGCGCTCCCAGGCCACTT
GAAGAGAGAGGGCGGGGCCGAGGGGCTGAGCCCGCGGGGGGAGGGAACAGCGTTGATCACGT
GACGTGGTTTCAGTGTTTACACCCGCAGCGGGCCGGGGGTTCGGCCTCAGTCAGGCGCTCAG
CTCCGTTTCGGTTTCACTTCCGGTGGAGGGCCGCCTCTGAGCGGGCGGCGGGCCGACGGCGA
GCGCGGGCGGCGGCGGTGACGGAGGCGCCGCTGCCAGGGGGCGTGCGGCAGCGCGGCGGCGG
CGGCGGCGGCGGCGGCGGCGGAGGCGGCGGCGGCGGCGGCGGCGGCGGCGGCTGGGCCTCGA
GCGCCCGCAGCCCACCTCTCGGGGGCGGGCTCCCGGCGCTAGCAGGGCTGAAGAGAAGATGG
AGGAGCTGGTGGTGGAAGTGCGGGGCTCCAATGGCGCTTTCTACAAGGCATTTGTAAAGGAT
GTTCATGAAGATTCAATAACAGTTGCATTTGAAAACAACTGGCAGCCTGATAGGCAGATTCC
ATTTCATGATGTCAGATTCCCACCTCCTGTAGGTTATAATAAAGATATAAATGAAAGTGATG
AAGTTGAGGTGTATTCCAGAGCAAATGAAAAAGAGCCTTGCTGTTGGTGGTTAGCTAAAGTG
AGGATGATAAAGGGTGAGTTTTATGTGATAGAATATGCAGCATGTGATGCAACTTACAATGA
AATTGTCACAATTGAACGTCTAAGATCTGTTAATCCCAACAAACCTGCCACAAAAGATACTT
TCCATAAGATCAAGCTGGATGTGCCAGAAGACTTACGGCAAATGTGTGCCAAAGAGGCGGCA
CATAAGGATTTTAAAAAGGCAGTTGGTGCCTTTTCTGTAACTTATGATCCAGAAAATTATCA
GCTTGTCATTTTGTCCATCAATGAAGTCACCTCAAAGCGAGCACATATGCTGATTGACATGC
ACTTTCGGAGTCTGCGCACTAAGTTGTCTCTGATAATGAGAAATGAAGAAGCTAGTAAGCAG
CTGGAGAGTTCAAGGCAGCTTGCCTCGAGATTTCATGAACAGTTTATCGTAAGAGAAGATCT
GATGGGTCTAGCTATTGGTACTCATGGTGCTAATATTCAGCAAGCTAGAAAAGTACCTGGGG
TCACTGCTATTGATCTAGATGAAGATACCTGCACATTTCATATTTATGGAGAGGATCAGGAT
GCAGTGAAAAAAGCTAGAAGTTTTCTCGAATTTGCTGAAGATGTAATACAAGTTCCAAGGAA
CTTAGTAGGCAAAGTAATAGGAAAAAATGGAAAGCTGATTCAGGAGATTGTGGACAAGTCAG
GAGTTGTGAGGGTGAGGATTGAGGCTGAAAATGAGAAAAATGTTCCACAAGAAGAGGAAATT
ATGCCACCAAATTCCCTTCCTTCCAATAATTCAAGGGTTGGACCTAATGCCCCAGAAGAAAA
AAAACATTTAGATATAAAGGAAAACAGCACCCATTTTTCTCAACCTAACAGTACAAAAGTCC
AGAGGGGTATGGTACCATTTGTTTTTGTGGGAACAAAGGACAGCATCGCTAATGCCACTGTT
CTTTTGGATTATCACCTGAACTATTTAAAGGAAGTAGACCAGTTGCGTTTGGAGAGATTACA
AATTGATGAGCAGTTGCGACAGATTGGAGCTAGTTCTAGACCACCACCAAATCGTACAGATA
AGGAAAAAAGCTATGTGACTGATGATGGTCAAGGAATGGGTCGAGGTAGTAGACCTTACAGA
AATAGGGGGCACGGCAGACGCGGTCCTGGATATACTTCAGGAACTAATTCTGAAGCATCAAA
TGCTTCTGAAACAGAATCTGACCACAGAGACGAACTCAGTGATTGGTCATTAGCTCCAACAG
AGGAAGAGAGGGAGAGCTTCCTGCGCAGAGGAGACGGACGGCGGCGTGGAGGGGGAGGAAGA
GGACAAGGAGGAAGAGGACGTGGAGGAGGCTTCAAAGGAAACGACGATCACTCCCGAACAGA
TAATCGTCCACGTAATCCAAGAGAGGCTAAAGGAAGAACAACAGATGGATCGCTTCAGATCA
GAGTTGACTGCAATAATGAAAGGAGTGTCCACACTAAAACATTACAGAATACCTCCAGTGAA
GGTAGTCGGCTGCGCACGGGTAAAGATCGTAACCAGAAGAAAGAGAAGCCAGACAGCGTGGA
TGGTCAGCAACCACTCGTGAATGGAGTACCCTGAGGATCCCCCAGTACATGACCTTATGGCC
TAGGGTCATTTCATGTCCTGTGTCAGTTTATGTTTTGGTCCACTTTTCCAGTATTTTAGTGG
ACCCTGAAATGTGTGTGATGTGACATTTGTCATTTTCATTAGCAAAAAAAGTTGTATGATCT
GTGCCTTTTTTATATCTTGGCAGGTAGGAATATTATATTTGGATGCAGAGTTCAGGGAAGAT
AAGTTGGAAACACTAAATGTTAAAGATGTAGCAAACCCTGTCAAACATTAGTACTTTATAGA
AGAATGCATGCTTTCCATATTTTTTTCCTTACATAAACATCAGGTTAGGCAGTATAAAGAAT
AGGACTTGTTTTTGTTTTTGTTTTGTTGCACTGAAGTTTGATAAATAGTGTTATTGAGAGAG
ATGTGTAATTTTTCTGTATAGACAGGAGAAGAAAGAACTATCTTCATCTGAGAGAGGCTAAA
ATGTTTTCAGCTAGGAACAAATCTTCCTGGTCGAAAGTTAGTAGGATATGCCTGCTCTTTGG
CCTGATGACCAATTTTAACTTAGAGCTTTTTTTTTTTAATTTTGTCTGCCCCAAGTTTTGTG
AAATTTTTCATATTTTAATTTCAACCTTATTTTGGAGAGATAGGAAGGTCATTTCCATGTAT
GCATAATAATCCTGCAAAGTACAGGTACTTTGTCTAAGAAACATTGGAAGCAGGTTAAATGT
TTTGTAAACTTTGAAATATATGGTCTAATGTTTAAGCAGAATTGGAAAAGACTAAGATCGGT
TAACAAATAACAACTTTTTTTTCTTTTTTTCTTTTGTTTTTTGAAGTGTTGGGGTTTGGTTT
TGTTTTTTGAGTCTTTTTTTTTTAAGTGAAATTTATTGAGGAAAAATATGTGAAGGACCTTC
ACTCTAAGATGTTATATTTTTCTTAAAAAGTAACTCCTAGTAGGGGTACCACTGAATCTGTA
CAGAGCCGTAAAAACTGAAGTTCTGCCTCTGATGTATTTTGTGAGTTTGTTTCTTTGAATTT
TCATTTTACAGTTACTTTTCCTTGCATACAAACAAGCATATAAAATGGCAACAAACTGCACA
TGATTTCACAAATATTAAAAAGTCTTTTAAAAAGTATTGCCAAACATTAATGTTGATTTCTA
GTTATTTATTCTGGGAATGTATAGTATTTGAAAACAGAAATTGGTACCTTGCACACATCATC
TGTAAGCTGTTTGGTTTTAAAATACTGTAGATAATTAACCAAGGTAGAATGACCTTGTAATG
TAACTGCTCTTGGGCAATATTCTCTGTACATATTAGCGACAACAGATTGGATTTTATGTTGA
CATTTGTTTGGTTATAGTGCAATATATTTTGTATGCAAGCAGTTTCAATAAAGTTTGATCTT
CCTCTGCTAAATTGATGTTGATGCAATCCTTACAAATGAT
>hFMP734-hFMR1-FMpA2 plasmid (SEQ ID NO: 10)
•hFMP734 promoter sequence in bold
•hFMR1 GOI sequence in bold and underlined
•FMpA2 sequence underlined
TGTGGGCGGACAAAATAGTTGGGAACTGGGAGGGGTGGAAATGGAGTTTTTAAGGATTATTT
AGGGAAGAGTGACAAAATAGATGGGAACTGGGTGTAGCGTCGTAAGCTAATACGAAAATTAA
AAATGACAAAATAGTTTGGAACTAGATTTCACTTATCTGGTTCGGATCTCCTAGAGCTTACA
TTATTGAAGCATTTATCAGGGTTATTGTCTCAGACCTGCAGGCAGCTGCGCGCTCGCTCGCT
CACTGAGGCCGCCCGGGCAAAGCCCGGGCGTCGGGCGACCTTTGGTCGCCCGGCCTCAGTGA
GCGAGCGAGCGCGCAGAGAGGGAGTGGCCAACTCCATCACTAGGGGTTCCTGCGGCCGCACG
CGTACTAGTTTCTGTCTAATCCTTCACCCCTATTCTCGCCTTCCACTCCACCTCCCGCTCAG
TCAGACTGCGCTACTTTGAACCGGACCAAACCAAACCAAACCAAACCAAACCAAACCAGACC
AGACACCCCCTCCCGCGGAATCCCAGAGAGGCCGAACTGGGATAACCGGATGCATTTGATTT
CCCACGCCACTGAGTGCACCTCTGCAGAAATGGGCGTTCTGGCCCTCGCGAGGCAGTGCGAC
CTGTCACCGCCCTTCAGCCTTCCCGCCCTCCACCAAGCCCGCGCACGCCCGGCCCGCGCGTC
TGTCTTTCGACCCGGCACCCCGGCCGGTTCCCAGCAGCGCGCATGCGCGCGCTCCCAGGCCA
CTTGAAGAGAGAGGGCGGGGCCGAGGGGCTGAGCCCGCGGGGGGAGGGAACAGCGTTGATCA
CGTGACGTGGTTTCAGTGTTTACACCCGCAGCGGGCCGGGGGTTCGGCCTCAGTCAGGCGCT
CAGCTCCGTTTCGGTTTCACTTCCGGTGGAGGGCCGCCTCTGAGCGGGCGGCGGGCCGACGG
CGAGCGCGGGCGGCGGCGGTGACGGAGGCGCCGCTGCCAGGGGGCGTGCGGCAGCGCGGCGG
CGGCGGCGGCGGCGGCGGCGGCGGAGGCGGCGGCGGCGGCGGCGGCGGCGGCGGCTGGGCCT
CGAGCGCCCGCAGCCCACCTCTCGGGGGCGGGCTCCCGGCGCTAGCAGGGCTGAAGAGAAGA
TGGAGGAGCTGGTGGTGGAAGTGCGGGGCTCCAATGGCGCTTTCTACAAGGCATTTGTAAAG
GATGTTCATGAAGATTCAATAACAGTTGCATTTGAAAACAACTGGCAGCCTGATAGGCAGAT
TCCATTTCATGATGTCAGATTCCCACCTCCTGTAGGTTATAATAAAGATATAAATGAAAGTG
ATGAAGTTGAGGTGTATTCCAGAGCAAATGAAAAAGAGCCTTGCTGTTGGTGGTTAGCTAAA
GTGAGGATGATAAAGGGTGAGTTTTATGTGATAGAATATGCAGCATGTGATGCAACTTACAA
TGAAATTGTCACAATTGAACGTCTAAGATCTGTTAATCCCAACAAACCTGCCACAAAAGATA
CTTTCCATAAGATCAAGCTGGATGTGCCAGAAGACTTACGGCAAATGTGTGCCAAAGAGGCG
GCACATAAGGATTTTAAAAAGGCAGTTGGTGCCTTTTCTGTAACTTATGATCCAGAAAATTA
TCAGCTTGTCATTTTGTCCATCAATGAAGTCACCTCAAAGCGAGCACATATGCTGATTGACA
TGCACTTTCGGAGTCTGCGCACTAAGTTGTCTCTGATAATGAGAAATGAAGAAGCTAGTAAG
CAGCTGGAGAGTTCAAGGCAGCTTGCCTCGAGATTTCATGAACAGTTTATCGTAAGAGAAGA
TCTGATGGGTCTAGCTATTGGTACTCATGGTGCTAATATTCAGCAAGCTAGAAAAGTACCTG
GGGTCACTGCTATTGATCTAGATGAAGATACCTGCACATTTCATATTTATGGAGAGGATCAG
GATGCAGTGAAAAAAGCTAGAAGTTTTCTCGAATTTGCTGAAGATGTAATACAAGTTCCAAG
GAACTTAGTAGGCAAAGTAATAGGAAAAAATGGAAAGCTGATTCAGGAGATTGTGGACAAGT
CAGGAGTTGTGAGGGTGAGGATTGAGGCTGAAAATGAGAAAAATGTTCCACAAGAAGAGGAA
ATTATGCCACCAAATTCCCTTCCTTCCAATAATTCAAGGGTTGGACCTAATGCCCCAGAAGA
AAAAAAACATTTAGATATAAAGGAAAACAGCACCCATTTTTCTCAACCTAACAGTACAAAAG
TCCAGAGGGGTATGGTACCATTTGTTTTTGTGGGAACAAAGGACAGCATCGCTAATGCCACT
GTTCTTTTGGATTATCACCTGAACTATTTAAAGGAAGTAGACCAGTTGCGTTTGGAGAGATT
ACAAATTGATGAGCAGTTGCGACAGATTGGAGCTAGTTCTAGACCACCACCAAATCGTACAG
ATAAGGAAAAAAGCTATGTGACTGATGATGGTCAAGGAATGGGTCGAGGTAGTAGACCTTAC
AGAAATAGGGGGCACGGCAGACGCGGTCCTGGATATACTTCAGGAACTAATTCTGAAGCATC
AAATGCTTCTGAAACAGAATCTGACCACAGAGACGAACTCAGTGATTGGTCATTAGCTCCAA
CAGAGGAAGAGAGGGAGAGCTTCCTGCGCAGAGGAGACGGACGGCGGCGTGGAGGGGGAGGA
AGAGGACAAGGAGGAAGAGGACGTGGAGGAGGCTTCAAAGGAAACGACGATCACTCCCGAAC
AGATAATCGTCCACGTAATCCAAGAGAGGCTAAAGGAAGAACAACAGATGGATCGCTTCAGA
TCAGAGTTGACTGCAATAATGAAAGGAGTGTCCACACTAAAACATTACAGAATACCTCCAGT
GAAGGTAGTCGGCTGCGCACGGGTAAAGATCGTAACCAGAAGAAAGAGAAGCCAGACAGCGT
GGATGGTCAGCAACCACTCGTGAATGGAGTACCCTGAGGATCCCCCAGTACATGACCTTATG
GCCTAGGGTCATTTCATGTCCTGTGTCAGTTTATGTTTTGGTCCACTTTTCCAGTATTTTAG
TGGACCCTGAAATGTGTGTGATGTGACATTTGTCATTTTCATTAGCAAAAAAAGTTGTATGA
TCTGTGCCTTTTTTATATCTTGGCAGGTAGGAATATTATATTTGGATGCAGAGTTCAGGGAA
GATAAGTTGGAAACACTAAATGTTAAAGATGTAGCAAACCCTGTCAAACATTAGTACTTTAT
AGAAGAATGCATGCTTTCCATATTTTTTTCCTTACATAAACATCAGGTTAGGCAGTATAAAG
AATAGGACTTGTTTTTGTTTTTGTTTTGTTGCACTGAAGTTTGATAAATAGTGTTATTGAGA
GAGATGTGTAATTTTTCTGTATAGACAGGAGAAGAAAGAACTATCTTCATCTGAGAGAGGCT
AAAATGTTTTCAGCTAGGAACAAATCTTCCTGGTCGAAAGTTAGTAGGATATGCCTGCTCTT
TGGCCTGATGACCAATTTTAACTTAGAGCTTTTTTTTTTTAATTTTGTCTGCCCCAAGTTTT
GTGAAATTTTTCATATTTTAATTTCAACCTTATTTTGGAGAGATAGGAAGGTCATTTCCATG
TATGCATAATAATCCTGCAAAGTACAGGTACTTTGTCTAAGAAACATTGGAAGCAGGTTAAA
TGTTTTGTAAACTTTGAAATATATGGTCTAATGTTTAAGCAGAATTGGAAAAGACTAAGATC
GGTTAACAAATAACAACTTTTTTTTCTTTTTTTCTTTTGTTTTTTGAAGTGTTGGGGTTTGG
TTTTGTTTTTTGAGTCTTTTTTTTTTAAGTGAAATTTATTGAGGAAAAATATGTGAAGGACC
TTCACTCTAAGATGTTATATTTTTCTTAAAAAGTAACTCCTAGTAGGGGTACCACTGAATCT
GTACAGAGCCGTAAAAACTGAAGTTCTGCCTCTGATGTATTTTGTGAGTTTGTTTCTTTGAA
TTTTCATTTTACAGTTACTTTTCCTTGCATACAAACAAGCATATAAAATGGCAACAAACTGC
ACATGATTTCACAAATATTAAAAAGTCTTTTAAAAAGTATTGCCAAACATTAATGTTGATTT
CTAGTTATTTATTCTGGGAATGTATAGTATTTGAAAACAGAAATTGGTACCTTGCACACATC
ATCTGTAAGCTGTTTGGTTTTAAAATACTGTAGATAATTAACCAAGGTAGAATGACCTTGTA
ATGTAACTGCTCTTGGGCAATATTCTCTGTACATATTAGCGACAACAGATTGGATTTTATGT
TGACATTTGTTTGGTTATAGTGCAATATATTTTGTATGCAAGCAGTTTCAATAAAGTTTGAT
CTTCCTCTGCTAAATTGATGTTGATGCAATCCTTACAAATGATTGCTTTTAAAATTTTAAGC
TAGGAAAAGAAATCTATAGAAAGTGTTCTGTTACAAAATGTAACTGTTACCATTGGAAATTT
CACGTCATAGGAAGTTAGCCTTTATCTACCAACTTTCAAGAACTTGTTTAATAAAGCGAAAA
ACTCAACCAAATGGTACAAAACCACAGTGTACCATTAAAATATGCACTAAGTCTCTTTTTTA
CAAAGGCTGTTCCGGACACGTGCGGACCGAGCGGCCGCAGGAACCCCTAGTGATGGAGTTGG
CCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGCTGCCTGCAGGCATGCAA
GCTGTAGCCAACCACTAGAACTATAGCTAGAGTCCTGGGCGAACAAACGATGCTCGCCTTCC
AGAAAACCGAGGATGCGAACCACTTCATCCGGGGTCAGCACCACCGGCAAGCGCCGCGACGG
CCGAGGTCTTCCGATCTCCTGAAGCCAGGGCAGATCCGTGCACAGCACCTTGCCGTAGAAGA
ACAGCAAGGCCGCCAATGCCTGACGATGCGTGGAGACCGAAACCTTGCGCTCGTTCGCCAGC
CAGGACAGAAATGCCTCGACTTCGCTGCTGCCCAAGGTTGCCGGGTGACGCACACCGTGGAA
ACGGATGAAGGCACGAACCCAGTTGACATAAGCCTGTTCGGTTCGTAAACTGTAATGCAAGT
AGCGTATGCGCTCACGCAACTGGTCCAGAACCTTGACCGAACGCAGCGGTGGTAACGGCGCA
GTGGCGGTTTTCATGGCTTGTTATGACTGTTTTTTTGTACAGTCTATGCCTCGGGCATCCAA
GCAGCAAGCGCGTTACGCCGTGGGTCGATGTTTGATGTTATGGAGCAGCAACGATGTTACGC
AGCAGGGCAGTCGCCCTAAAACAAAGTTAGGTGGCTCAAGTATGGGCATCATTCGCACATGT
AGGCTCGGCCCTGACCAAGTCAAATCCATGCGGGCTGCTCTTGATCTTTTCGGTCGTGAGTT
CGGAGACGTAGCCACCTACTCCCAACATCAGCCGGACTCCGATTACCTCGGGAACTTGCTCC
GTAGTAAGACATTCATCGCGCTTGCTGCCTTCGACCAAGAAGCGGTTGTTGGCGCTCTCGCG
GCTTACGTTCTGCCCAAGTTTGAGCAGCCGCGTAGTGAGATCTATATCTATGATCTCGCAGT
CTCCGGCGAGCACCGGAGGCAGGGCATTGCCACCGCGCTCATCAATCTCCTCAAGCATGAGG
CCAACGCGCTTGGTGCTTATGTGATCTACGTGCAAGCAGATTACGGTGACGATCCCGCAGTG
GCTCTCTATACAAAGTTGGGCATACGGGAAGAAGTGATGCACTTTGATATCGACCCAAGTAC
CGCCACCTAACAATTCGTTCAAGCCGAGATCGGCTTCCCGGCCGCGGAGTTGTTCGGTAAAT
TGTCACAACGCCGCGAATATAGTCTTTACCATGCCCTTGGCCACGCCCCTCTTTAATACGAC
GGGCAATTTGCACTTCAGAAAATGAAGAGTTTGCTTTAGCCATAACAAAAGTCCAGTATGCT
TTTTCACAGCATAACTGGACTGATTTCAGTTTACAACTATTCTGTCTAGTTTAAGACTTTAT
TGTCATAGTTTAGATCTATTTTGTTCAGTTTAAGACTTTATTGTCCGCCCACACCCGCTTAC
GCAGGGCATCCATTTATTACTCAACCGTAACCGATTTTGCCAGGTTACGCGGCTGGTCTATG
CGGTGTGAAATACCGCACAGATGCGTAAGGAGAAAATACCGCATCAGGCGCTCTTCCGCTTC
CTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGACAGGAC
TATAAAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAG
TTACCAATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCCCCAGT
GCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATAC
GGGATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTCGTGC
ACCCAACTGATCTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATACTCATACTCTTC
CTTTTTCAATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGTGCCACCTA
AATTGTAAGCGTTAATATTTTGTTAAAATTCGCGTTAAATTTTTGTTAAATCAGCTCATTTT
TTAACCAATAGGCCGAAATCGGCAAAATCCCTTATAAATCAAAAGAATAGACCGAGATAGGG
TTGAGTGTTGTTCCAGTTTGGAACAAGAGTCCACTATTAAGAACGTGGACTCCAACGTCAAA
GGGCGAAAAACCGTCTATCAGGGCGATGGCCCACTACGTGAACCATCACCCTAATCAAGTTT
TTTGGGGTCGAGGTGCCGTAAAGCACTAAATCGGAACCCTAAAGGGAGCCCCCGATTTAGAG
CTTGACGGGGAAAGCCGGCGAACGTGGCGAGAAAGGAAGGGAAGAAAGCGAAAGGAGCGGGC
GCTAGGGCGCTGGCAAGTGTAGCGGTCACGCTGCGCGTAACCACCACACCCGCCGCGCTTAA
TGCGCCGCTACAGGGCGCGTCCCATTCGCCATTCAGGCTGCAAATAAGCGTTGATATTCAGT
CAATTACAAACATTAATAACGAAGAGATGACAGAAAAATTTTCATTCTGTGACAGAGAAAAA
GTAGCCGAAGATGACGGTTTGTCACATGGAGTTGGCAGGATGTTTGATTAAAAACATAACAG
GAAGAAAAATGCCCCGC
>hFMP734-hFMR1-FMpA2 (SEQ ID NO: 11)
TTCTGTCTAATCCTTCACCCCTATTCTCGCCTTCCACTCCACCTCCCGCTCAGTCAGACTGC
GCTACTTTGAACCGGACCAAACCAAACCAAACCAAACCAAACCAAACCAGACCAGACACCCC
CTCCCGCGGAATCCCAGAGAGGCCGAACTGGGATAACCGGATGCATTTGATTTCCCACGCCA
CTGAGTGCACCTCTGCAGAAATGGGCGTTCTGGCCCTCGCGAGGCAGTGCGACCTGTCACCG
CCCTTCAGCCTTCCCGCCCTCCACCAAGCCCGCGCACGCCCGGCCCGCGCGTCTGTCTTTCG
ACCCGGCACCCCGGCCGGTTCCCAGCAGCGCGCATGCGCGCGCTCCCAGGCCACTTGAAGAG
AGAGGGCGGGGCCGAGGGGCTGAGCCCGCGGGGGGAGGGAACAGCGTTGATCACGTGACGTG
GTTTCAGTGTTTACACCCGCAGCGGGCCGGGGGTTCGGCCTCAGTCAGGCGCTCAGCTCCGT
TTCGGTTTCACTTCCGGTGGAGGGCCGCCTCTGAGCGGGCGGCGGGCCGACGGCGAGCGCGG
GCGGCGGCGGTGACGGAGGCGCCGCTGCCAGGGGGCGTGCGGCAGCGCGGCGGCGGCGGCGG
CGGCGGCGGCGGCGGAGGCGGCGGCGGCGGCGGCGGCGGCGGCGGCTGGGCCTCGAGCGCCC
GCAGCCCACCTCTCGGGGGCGGGCTCCCGGCGCTAGCAGGGCTGAAGAGAAGATGGAGGAGC
TGGTGGTGGAAGTGCGGGGCTCCAATGGCGCTTTCTACAAGGCATTTGTAAAGGATGTTCAT
GAAGATTCAATAACAGTTGCATTTGAAAACAACTGGCAGCCTGATAGGCAGATTCCATTTCA
TGATGTCAGATTCCCACCTCCTGTAGGTTATAATAAAGATATAAATGAAAGTGATGAAGTTG
AGGTGTATTCCAGAGCAAATGAAAAAGAGCCTTGCTGTTGGTGGTTAGCTAAAGTGAGGATG
ATAAAGGGTGAGTTTTATGTGATAGAATATGCAGCATGTGATGCAACTTACAATGAAATTGT
CACAATTGAACGTCTAAGATCTGTTAATCCCAACAAACCTGCCACAAAAGATACTTTCCATA
AGATCAAGCTGGATGTGCCAGAAGACTTACGGCAAATGTGTGCCAAAGAGGCGGCACATAAG
GATTTTAAAAAGGCAGTTGGTGCCTTTTCTGTAACTTATGATCCAGAAAATTATCAGCTTGT
CATTTTGTCCATCAATGAAGTCACCTCAAAGCGAGCACATATGCTGATTGACATGCACTTTC
GGAGTCTGCGCACTAAGTTGTCTCTGATAATGAGAAATGAAGAAGCTAGTAAGCAGCTGGAG
AGTTCAAGGCAGCTTGCCTCGAGATTTCATGAACAGTTTATCGTAAGAGAAGATCTGATGGG
TCTAGCTATTGGTACTCATGGTGCTAATATTCAGCAAGCTAGAAAAGTACCTGGGGTCACTG
CTATTGATCTAGATGAAGATACCTGCACATTTCATATTTATGGAGAGGATCAGGATGCAGTG
AAAAAAGCTAGAAGTTTTCTCGAATTTGCTGAAGATGTAATACAAGTTCCAAGGAACTTAGT
AGGCAAAGTAATAGGAAAAAATGGAAAGCTGATTCAGGAGATTGTGGACAAGTCAGGAGTTG
TGAGGGTGAGGATTGAGGCTGAAAATGAGAAAAATGTTCCACAAGAAGAGGAAATTATGCCA
CCAAATTCCCTTCCTTCCAATAATTCAAGGGTTGGACCTAATGCCCCAGAAGAAAAAAAACA
TTTAGATATAAAGGAAAACAGCACCCATTTTTCTCAACCTAACAGTACAAAAGTCCAGAGGG
GTATGGTACCATTTGTTTTTGTGGGAACAAAGGACAGCATCGCTAATGCCACTGTTCTTTTG
GATTATCACCTGAACTATTTAAAGGAAGTAGACCAGTTGCGTTTGGAGAGATTACAAATTGA
TGAGCAGTTGCGACAGATTGGAGCTAGTTCTAGACCACCACCAAATCGTACAGATAAGGAAA
AAAGCTATGTGACTGATGATGGTCAAGGAATGGGTCGAGGTAGTAGACCTTACAGAAATAGG
GGGCACGGCAGACGCGGTCCTGGATATACTTCAGGAACTAATTCTGAAGCATCAAATGCTTC
TGAAACAGAATCTGACCACAGAGACGAACTCAGTGATTGGTCATTAGCTCCAACAGAGGAAG
AGAGGGAGAGCTTCCTGCGCAGAGGAGACGGACGGCGGCGTGGAGGGGGAGGAAGAGGACAA
GGAGGAAGAGGACGTGGAGGAGGCTTCAAAGGAAACGACGATCACTCCCGAACAGATAATCG
TCCACGTAATCCAAGAGAGGCTAAAGGAAGAACAACAGATGGATCGCTTCAGATCAGAGTTG
ACTGCAATAATGAAAGGAGTGTCCACACTAAAACATTACAGAATACCTCCAGTGAAGGTAGT
CGGCTGCGCACGGGTAAAGATCGTAACCAGAAGAAAGAGAAGCCAGACAGCGTGGATGGTCA
GCAACCACTCGTGAATGGAGTACCCTGAGGATCCCCCAGTACATGACCTTATGGCCTAGGGT
CATTTCATGTCCTGTGTCAGTTTATGTTTTGGTCCACTTTTCCAGTATTTTAGTGGACCCTG
AAATGTGTGTGATGTGACATTTGTCATTTTCATTAGCAAAAAAAGTTGTATGATCTGTGCCT
TTTTTATATCTTGGCAGGTAGGAATATTATATTTGGATGCAGAGTTCAGGGAAGATAAGTTG
GAAACACTAAATGTTAAAGATGTAGCAAACCCTGTCAAACATTAGTACTTTATAGAAGAATG
CATGCTTTCCATATTTTTTTCCTTACATAAACATCAGGTTAGGCAGTATAAAGAATAGGACT
TGTTTTTGTTTTTGTTTTGTTGCACTGAAGTTTGATAAATAGTGTTATTGAGAGAGATGTGT
AATTTTTCTGTATAGACAGGAGAAGAAAGAACTATCTTCATCTGAGAGAGGCTAAAATGTTT
TCAGCTAGGAACAAATCTTCCTGGTCGAAAGTTAGTAGGATATGCCTGCTCTTTGGCCTGAT
GACCAATTTTAACTTAGAGCTTTTTTTTTTTAATTTTGTCTGCCCCAAGTTTTGTGAAATTT
TTCATATTTTAATTTCAACCTTATTTTGGAGAGATAGGAAGGTCATTTCCATGTATGCATAA
TAATCCTGCAAAGTACAGGTACTTTGTCTAAGAAACATTGGAAGCAGGTTAAATGTTTTGTA
AACTTTGAAATATATGGTCTAATGTTTAAGCAGAATTGGAAAAGACTAAGATCGGTTAACAA
ATAACAACTTTTTTTTCTTTTTTTCTTTTGTTTTTTGAAGTGTTGGGGTTTGGTTTTGTTTT
TTGAGTCTTTTTTTTTTAAGTGAAATTTATTGAGGAAAAATATGTGAAGGACCTTCACTCTA
AGATGTTATATTTTTCTTAAAAAGTAACTCCTAGTAGGGGTACCACTGAATCTGTACAGAGC
CGTAAAAACTGAAGTTCTGCCTCTGATGTATTTTGTGAGTTTGTTTCTTTGAATTTTCATTT
TACAGTTACTTTTCCTTGCATACAAACAAGCATATAAAATGGCAACAAACTGCACATGATTT
CACAAATATTAAAAAGTCTTTTAAAAAGTATTGCCAAACATTAATGTTGATTTCTAGTTATT
TATTCTGGGAATGTATAGTATTTGAAAACAGAAATTGGTACCTTGCACACATCATCTGTAAG
CTGTTTGGTTTTAAAATACTGTAGATAATTAACCAAGGTAGAATGACCTTGTAATGTAACTG
CTCTTGGGCAATATTCTCTGTACATATTAGCGACAACAGATTGGATTTTATGTTGACATTTG
TTTGGTTATAGTGCAATATATTTTGTATGCAAGCAGTTTCAATAAAGTTTGATCTTCCTCTG
CTAAATTGATGTTGATGCAATCCTTACAAATGATTGCTTTTAAAATTTTAAGCTAGGAAAAG
AAATCTATAGAAAGTGTTCTGTTACAAAATGTAACTGTTACCATTGGAAATTTCACGTCATA
GGAAGTTAGCCTTTATCTACCAACTTTCAAGAACTTGTTTAATAAAGCGAAAAACTCAACCA
AATGGTACAAAACCACAGTGTACCATTAAAATATGCACTAAGTCTCTTTTTTACAAAGGCTG
T
>hFMP734, human core promoter (SEQ ID NO: 12)
TTCTGTCTAATCCTTCACCCCTATTCTCGCCTTCCACTCCACCTCCCGCTCAGTCAGACTGC
GCTACTTTGAACCGGACCAAACCAAACCAAACCAAACCAAACCAAACCAGACCAGACACCCC
CTCCCGCGGAATCCCAGAGAGGCCGAACTGGGATAACCGGATGCATTTGATTTCCCACGCCA
CTGAGTGCACCTCTGCAGAAATGGGCGTTCTGGCCCTCGCGAGGCAGTGCGACCTGTCACCG
CCCTTCAGCCTTCCCGCCCTCCACCAAGCCCGCGCACGCCCGGCCCGCGCGTCTGTCTTTCG
ACCCGGCACCCCGGCCGGTTCCCAGCAGCGCGCATGCGCGCGCTCCCAGGCCACTTGAAGAG
AGAGGGCGGGGCCGAGGGGCTGAGCCCGCGGGGGGAGGGAACAGCGTTGATCACGTGACGTG
GTTTCAGTGTTTACACCCGCAGCGGGCCGGGGGTTCGGCCTCAGTCAGGCGCTCAGCTCCGT
TTCGGTTTCACTTCCGGTGGAGGGCCGCCTCTGAGCGGGCGGCGGGCCGACGGCGAGCGCGG
GCGGCGGCGGTGACGGAGGCGCCGCTGCCAGGGGGCGTGCGGCAGCGCGGCGGCGGCGGCGG
CGGCGGCGGCGGCGGAGGCGGCGGCGGCGGCGGCGGCGGCGGCGGCTGGGCCTCGAGCGCCC
GCAGCCCACCTCTCGGGGGCGGGCTCCCGGCGCTAGCAGGGCTGAAGAGAAG
>Endogenous human FMRI 3′UTR and polyA (FMpA2) (SEQ ID NO: 13)
GTCATTTCATGTCCTGTGTCAGTTTATGTTTTGGTCCACTTTTCCAGTATTTTAGTGGACCC
TGAAATGTGTGTGATGTGACATTTGTCATTTTCATTAGCAAAAAAAGTTGTATGATCTGTGC
CTTTTTTATATCTTGGCAGGTAGGAATATTATATTTGGATGCAGAGTTCAGGGAAGATAAGT
TGGAAACACTAAATGTTAAAGATGTAGCAAACCCTGTCAAACATTAGTACTTTATAGAAGAA
TGCATGCTTTCCATATTTTTTTCCTTACATAAACATCAGGTTAGGCAGTATAAAGAATAGGA
CTTGTTTTTGTTTTTGTTTTGTTGCACTGAAGTTTGATAAATAGTGTTATTGAGAGAGATGT
GTAATTTTTCTGTATAGACAGGAGAAGAAAGAACTATCTTCATCTGAGAGAGGCTAAAATGT
TTTCAGCTAGGAACAAATCTTCCTGGTCGAAAGTTAGTAGGATATGCCTGCTCTTTGGCCTG
ATGACCAATTTTAACTTAGAGCTTTTTTTTTTTAATTTTGTCTGCCCCAAGTTTTGTGAAAT
TTTTCATATTTTAATTTCAACCTTATTTTGGAGAGATAGGAAGGTCATTTCCATGTATGCAT
AATAATCCTGCAAAGTACAGGTACTTTGTCTAAGAAACATTGGAAGCAGGTTAAATGTTTTG
TAAACTTTGAAATATATGGTCTAATGTTTAAGCAGAATTGGAAAAGACTAAGATCGGTTAAC
AAATAACAACTTTTTTTTCTTTTTTTCTTTTGTTTTTTGAAGTGTTGGGGTTTGGTTTTGTT
TTTTGAGTCTTTTTTTTTTAAGTGAAATTTATTGAGGAAAAATATGTGAAGGACCTTCACTC
TAAGATGTTATATTTTTCTTAAAAAGTAACTCCTAGTAGGGGTACCACTGAATCTGTACAGA
GCCGTAAAAACTGAAGTTCTGCCTCTGATGTATTTTGTGAGTTTGTTTCTTTGAATTTTCAT
TTTACAGTTACTTTTCCTTGCATACAAACAAGCATATAAAATGGCAACAAACTGCACATGAT
TTCACAAATATTAAAAAGTCTTTTAAAAAGTATTGCCAAACATTAATGTTGATTTCTAGTTA
TTTATTCTGGGAATGTATAGTATTTGAAAACAGAAATTGGTACCTTGCACACATCATCTGTA
AGCTGTTTGGTTTTAAAATACTGTAGATAATTAACCAAGGTAGAATGACCTTGTAATGTAAC
TGCTCTTGGGCAATATTCTCTGTACATATTAGCGACAACAGATTGGATTTTATGTTGACATT
TGTTTGGTTATAGTGCAATATATTTTGTATGCAAGCAGTTTCAATAAAGTTTGATCTTCCTC
TGCTAAATTGATGTTGATGCAATCCTTACAAATGATTGCTTTTAAAATTTTAAGCTAGGAAA
AGAAATCTATAGAAAGTGTTCTGTTACAAAATGTAACTGTTACCATTGGAAATTTCACGTCA
TAGGAAGTTAGCCTTTATCTACCAACTTTCAAGAACTTGTTTAATAAAGCGAAAAACTCAAC
CAAATGGTACAAAACCACAGTGTACCATTAAAATATGCACTAAGTCTCTTTTTTACAAAGGC
TGT

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

1. A therapeutic polynucleotide construct comprising:
a human endogenous FMR1 promoter fragment; a human FMR1 coding sequence, an endogenous human 3′ regulatory element, and a polyadenylation signal.

2. The polynucleotide of claim 1, wherein the human FMR1 coding sequence comprises any of the following sequences: a sequence having at least 80% identity to SEQ ID NO: 4, a sequence having at least 90% identity to SEQ ID NO:4, a sequence having at least 95% identity to SEQ ID NO:4, SEQ ID NO:4, or FMR1 isoform 7 sequence.

3. The polynucleotide of claim 1, wherein the human endogenous FMR1 promoter fragment comprises any of the following nucleotide sequences: a nucleotide sequence having at least 80% identity to SEQ ID NO:3 or to SEQ ID NO: 12, a nucleotide sequence having at least 90% identity to SEQ ID NO:3 or to SEQ ID NO: 12, a nucleotide sequence having at least 95% identity to SEQ ID NO:3 or to SEQ ID NO: 12, SEQ ID NO:3 or SEQ ID NO:12.

4. The polynucleotide of claim 1, wherein the 3′regulatory element comprises any of the following nucleotide sequences: a nucleotide sequence having at least 80% identity to SEQ ID NO:5 or to SEQ ID NO:13, a nucleotide sequence having at least 90% identity to SEQ ID NO:5 or to SEQ ID NO:13, a nucleotide sequence having at least 95% identity to SEQ ID NO:5 or to SEQ ID NO:13, SEQ ID NO:5 or SEQ ID NO:13.

5. The polynucleotide of claim 1, wherein the polynucleotide construct comprises any of the following sequences: a polynucleotide sequence having at least 90% identity to SEQ ID NO:2, a polynucleotide sequence having at least 90% identity to SEQ ID NO:9, a polynucleotide sequence having at least 90% identity to SEQ ID NO: 11, SEQ ID NO:2, SEQ ID NO:9 or SEQ ID NO:11.

6. The polynucleotide of claim 1, further comprising at least one adeno-associated virus (AAV) inverted terminal repeat (ITR).

7. The polynucleotide of claim 6, wherein the polynucleotide comprises two AAV ITRs.

8. A vector comprising the polynucleotide of claim 1.

9. The vector of claim 8, wherein the vector is a viral vector.

10. The vector of claim 8, wherein the vector is an adeno-associated virus (AAV) vector.

11. The vector of claim 10, wherein the AAV vector is an AAV9 vector.

12. A recombinant adeno-associated virus (rAAV), comprising the polynucleotide of claim 1.

13. The rAAV of claim 12, wherein the rAAV is AAV9.

14. A virion comprising the rAAV of claim 12.

15. A transformed cell comprising the polynucleotide of claim 1.

16. A pharmaceutical composition comprising the polynucleotide of claim 1 and a pharmaceutically acceptable carrier.

17. A method of treating Fragile X syndrome and related disorders in a subject, the method comprising administering to the subject an effective amount of the polynucleotide of claim 1.

18. The method of claim 17, wherein the administration is by intracerebral ventricular injection or by intracisternal magna administration to the subject.

19. The polynucleotide of claim 1 for use in a method of treating Fragile X syndrome and related disorders in a subject.

20. The polynucleotide, vector, rAAV, virion or pharmaceutical composition for use of claim 19 wherein the polynucleotide, vector, rAAV, virion or pharmaceutical composition is administered to the subject by intracerebral ventricular injection or by intracisternal magna administration.
